(19) United States
(12) Patent Application Publication
(10) Pub. No.: US 2013/0045948 A1
(43) Pub. Date: Feb. 21, 2013

(54) AZOCYCLIC INHIBITORS OF FATTY ACID AMIDE HYDROLASE

(71) Applicants: Mei H. Dung, Garnet Valley, PA (US); Robert James Pasteris, Newark, DE (US)

(72) Inventors: Mei H. Dung, Garnet Valley, PA (US); Robert James Pasteris, Newark, DE (US)

(73) Assignee: E I DU PONT DE NEMOURS AND COMPANY, Wilmington, DE (US)

(21) Appl. No.: 13/515,010

(22) PCT Filed: Dec. 10, 2010
(86) PCT No.: PCT/US2010/059850

§ 371 Date: Aug. 29, 2012

Related U.S. Application Data
(60) Provisional application No. 61/285,600, filed on Dec. 11, 2009.

(51) Int. Cl. A61K 31/454; C07F 7/10; A61K 31/695; A61K 31/4545; A61P 25/24; A61K 31/496; A61P 25/00; A61P 29/00; A61P 25/22; C07D 417/14; A61K 31/506

(52) U.S. Cl. 514/63; 546/209; 514/326; 546/14; 546/194; 514/318; 544/316; 514/274; 544/319; 544/369; 514/254.02; 544/229; 514/269; 546/198; 514/321; 544/331; 514/275; 544/328; 514/256

(57) ABSTRACT

Disclosed are compounds of Formula 1, including all stereoisomers, N-oxides, and salts thereof,

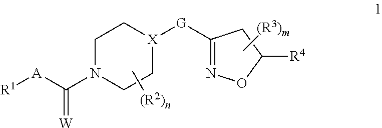

wherein
A, W, X, G, R¹, R², R³, R⁴, m and n are as defined in the disclosure.
   Also disclosed are pharmaceutical compositions containing the compounds of Formula 1 and methods for treating a disease or condition mediated by fatty acid amide hydrolase activity comprising applying a therapeutically effective amount of a compound or a composition of the invention.

### FIELD OF THE INVENTION

This invention relates to certain isoxazolyl-substituted piperidine and piperazine urea and carbamate compounds, their N-oxides and the pharmaceutically acceptable salts of such compounds. The invention also relates to compositions containing the compounds and the uses of the compounds in treating diseases or conditions associated with fatty acid amide hydrolase activity.

### BACKGROUND OF THE INVENTION

Fatty acid amides represent a class of signaling lipids with diverse cellular and physiological effects. Fatty acid amides are hydrolyzed to their corresponding fatty acids by an enzyme known as fatty acid amide hydrolase (FAAH). FAAH is a mammalian integral membrane serine hydrolase responsible for the hydrolysis of a number of primary and secondary fatty acid amides, including the neuromodulatory compounds anandamide and oleamide. Anandamide has been shown to possess cannabinoid-like analgesic properties and is released by stimulated neurons. The effects and endogenous levels of anandamide increase with pain stimulation, implying it has a role in suppressing pain neurotransmission and behavioral analgesia. Small-molecule FAAH inhibitors that elevate brain anandamide levels have demonstrated efficacy in animal models of pain, inflammation, anxiety and depression. Further description of FAAH inhibitors and methods of evaluating their activity can be found in A. H. Lichtman et al. J. Pharmacol. Exp. Ther. 2004, 311(2), 441-448; A. Jayamanne et al. Br. J. Pharmacol. 2006, 147(3), 281-288; S. Kathuria et al. Nature Med. 2003, 9(1), 76-81; and D. Piomelli et al. Proc. Natl. Acad. Sci. 2005, 102(51), 18620-18625.

There remains a need for new compounds that are inhibitors of FAAH and are useful in the treatment of a wide range of diseases, disorders and conditions, including pain.

### SUMMARY OF THE INVENTION

This invention relates to compounds of Formula 1 (including all stereoisomers), N-oxides, and salts thereof:

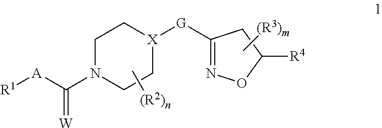

wherein
A is O, S or NR⁶;
W is O or S;
X is CR^{2a} or N;
R¹ is phenyl, naphthalenyl or 1,2-benzisoxazol-3-yl, each optionally substituted with up to 3 substituents independently selected from R^{5a}; or a 5- to 6-membered heteroaromatic ring, the ring containing ring members selected from carbon atoms and 1 to 4 heteroatoms independently selected from up to 2 O, up to 2 S and up to 4 N atoms, the ring optionally substituted with up to 3 substituents independently selected from R^{5a} on carbon atom ring members and R^{5b} on nitrogen atom ring members;
each R² is independently halogen, cyano, hydroxy, C₁-C₂ alkyl, C₁-C₂ haloalkyl or C₁-C₂ alkoxy;
R^{2a} is H, halogen, cyano, hydroxy, C₁-C₂ alkyl, C₁-C₂ haloalkyl or C₁-C₂ alkoxy;
each R³ is independently halogen, cyano, C₁-C₃ alkyl or C₁-C₃ haloalkyl;
R⁴ is C₁-C₈ alkyl, C₁-C₈ haloalkyl, C₃-C₈ cycloalkyl, C₃-C₈ halocycloalkyl, C₄-C₁₀ alkylcycloalkyl, C₄-C₁₀ cycloalkylalkyl, C₂-C₈ alkoxyalkyl, C₂-C₈ haloalkoxyalkyl, C₄-C₁₀ cycloalkoxyalkyl, C₃-C₈ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₂-C₆ haloalkylaminoalkyl, C₃-C₈ dialkylaminoalkyl, C₄-C₁₀ cycloalkylaminoalkyl, C₁-C₆ hydroxyalkyl, C₂-C₆ alkylcarbonyl, C₂-C₆ haloalkylcarbonyl, C₂-C₆ alkoxycarbonyl, C₂-C₆ alkylaminocarbonyl or C₃-C₈ dialkylaminocarbonyl; or benzyl, phenyl, naphthalenyl, 1,3-dihydro-1,3-dioxo-2H-isoindol-2-yl, 2-oxo-3(2H)-benzooxazol-3-yl or 2-oxo-3(2H)-benzothiazol-3-yl or each optionally substituted with up to 3 substituents independently selected from R^{8a}; or a 5- to 6-membered heteroaromatic ring, the ring optionally substituted with up to 3 substituents independently selected from R^{8a} on carbon atom ring members and R^{8b} on nitrogen atom ring members;
each R^{5a} is independently halogen, hydroxy, amino, cyano, nitro, C₁-C₄ alkyl, C₁-C₆ haloalkyl, C₃-C₆ cycloalkyl, C₃-C₆ halocycloalkyl, C₂-C₄ alkoxyalkyl, C₁-C₄ hydroxyalkyl, C₁-C₄ alkoxy, C₁-C₄ haloalkoxy, C₁-C₄ alkylthio, C₁-C₄ haloalkylthio, C₁-C₄ alkylsulfinyl, C₁-C₄ alkylsulfonyl, C₁-C₄ haloalkylsulfinyl, C₁-C₄ haloalkylsulfonyl, C₁-C₄ alkylamino, C₂-C₈ dialkylamino, C₂-C₄ alkylcarbonyl, C₂-C₆ alkoxycarbonyl, C₂-C₆ alkylaminocarbonyl, C₃-C₈ dialkylaminocarbonyl, C₂-C₆ alkylcarbonyloxy, C₂-C₆ alkylcarbonylthio or C₃-C₆ trialkylsilyl;
each R^{5b} is independently C₁-C₄ alkyl, C₃-C₄ alkenyl, C₃-C₄ alkynyl, C₃-C₆ cycloalkyl, C₁-C₄ haloalkyl, C₃-C₄ haloalkenyl, C₃-C₄ haloalkynyl, C₃-C₆ halocycloalkyl or C₂-C₄ alkoxyalkyl;
R⁶ is H, C₁-C₄ alkyl, C₂-C₄ alkenyl, C₂-C₄ alkynyl, C₁-C₄ haloalkyl, C₂-C₄ haloalkenyl, C₂-C₄ haloalkynyl, C₂-C₄ alkoxyalkyl, C₂-C₄ alkylcarbonyl, C₂-C₄ haloalkylcarbonyl, C₁-C₄ alkylsulfonyl or C₁-C₄ haloalkylsulfonyl;
G is a 5-membered heteroaromatic ring, the ring containing ring members selected from carbon atoms and 1 to 3 heteroatoms independently selected from up to 2 O, up to 2 S and up to 3 N atoms, the ring optionally substituted with up to 1 substituent selected from R^{7a} on a carbon atom and R^{7b} on a nitrogen atom;
R^{7a} is halogen, cyano, C₁-C₂ alkyl or C₁-C₂ haloalkyl;
R^{7b} is C₁-C₂ alkyl or C₁-C₂ haloalkyl;
each R^{8a} is independently halogen, hydroxy, amino, cyano, nitro, C₁-C₄ alkyl, C₁-C₄ haloalkyl, C₁-C₄ alkoxy, C₁-C₄ haloalkoxy, C₁-C₄ alkylthio, C₁-C₄ haloalkylthio, C₁-C₄ alkylsulfinyl, C₁-C₄ alkylsulfonyl, C₁-C₄ haloalkylsulfinyl, C₁-C₄ haloalkylsulfonyl, C₁-C₄ alkylamino, C₂-C₆ dialkylamino, C₂-C₄ alkylcarbonyl, C₂-C₆ alkoxycarbonyl, C₂-C₆ alkylaminocarbonyl or C₃-C₈ dialkylaminocarbonyl; or
a pair of R^{8a} and R³ are taken together with the atoms to which they are attached to form a 5- to 7-membered ring, the ring containing ring members selected from carbon atoms and up to 2 heteroatoms independently selected from up to 1 O, up to 1 S and up to 1 N, wherein up to 2 carbon atom ring members are independently selected from C(═O) and C(═S), and the sulfur atom ring members are independently selected from S(═O)ᵤ(═NR¹⁰)_{z}, the ring optionally substituted with up to 2 substituents independently selected from R^{9a} on carbon atom ring members and from R^{9b} on a nitrogen atom ring member;
each R^{8b} is independently C₁-C₄ alkyl or C₁-C₄ haloalkyl; or
a pair of R^{8b} and R³ are taken together with the atoms to which they are attached to form a 5- to 7-membered ring, the ring containing ring members selected from carbon atoms and up to 2 heteroatoms independently selected from up to 1 O, up to 1 S and up to 1 N, wherein up to 2 carbon atom ring members are independently selected from C(═O) and C(═S), and the sulfur atom ring members are independently selected from S(═O)ᵤ(═NR¹⁰)_{z}, the ring optionally substituted with up to 2 substituents independently selected from R^{9a} on carbon atom ring members and from R^{9b} on a nitrogen atom ring member;
each R^{9a} is independently halogen, C₁-C₄ alkyl, C₁-C₄ haloalkyl, C₁-C₄ alkoxy, C₁-C₄ haloalkoxy, C₁-C₄ alkylthio or C₁-C₄ haloalkylthio;
R^{9b} is C₁-C₄ alkyl or C₁-C₄ haloalkyl;
R¹⁰ is independently H, C₁-C₄ alkyl, C₂-C₄ alkenyl, C₂-C₄ alkynyl, C₁-C₄ haloalkyl, C₂-C₄ haloalkenyl, C₂-C₄ haloalkynyl, C₂-C₄ alkoxyalkyl, C₂-C₄ alkylcarbonyl, C₂-C₄ haloalkylcarbonyl, C₁-C₄ alkylsulfonyl or C₁-C₄ haloalkylsulfonyl;
m is 0, 1 or 2;
n is 0, 1 or 2; and
u and z in the instance of S(═O)ᵤ(═NR¹⁰)_{z} are independently 0, 1 or 2, provided that the sum of u and z in the instance of S(═O)ᵤ(═NR¹⁰)_{z} is 0, 1 or 2;
provided that when X is N, then G is attached to X through a carbon atom ring member.

This invention also relates to pharmaceutical compositions comprising a therapeutically effective amount of a compound of Formula 1, an N-oxide or a pharmaceutically acceptable salt thereof and a pharmaceutically acceptable carrier and optionally a further therapeutic agent.

This invention is also directed to methods of inhibiting fatty acid amide hydrolase activity comprising administering to a subject a compound of Formula 1, an N-oxide or a pharmaceutically acceptable salt thereof to achieve a serum concentration sufficient to inhibit fatty acid amide hydrolase activity in the subject.

This invention is also directed to methods of treating diseases, disorders or conditions including acute pain, chronic pain, neuropathic pain, nociceptive pain, inflammatory pain, urinary incontinence, overactive bladder, emesis, cognitive disorders, anxiety, depression, sleeping disorders, eating disorders, movement disorders, glaucoma, psoriasis, multiple sclerosis, cerebrovascular disorders, brain injury, gastrointestinal disorders, hypertension, or cardiovascular disease in a subject comprising administering to the subject a therapeutically effective amount of an inhibitor of fatty acid amide hydrolase selected from compounds of Formula 1, N-oxides or pharmaceutically acceptable salts thereof.

This invention is also directed to pharmaceutical compositions comprising a therapeutically effective amount of a compound of Formula 1, an N-oxide or a pharmaceutically acceptable salt thereof for use in treating FAAH-mediated diseases, disorders or conditions including acute pain, chronic pain, neuropathic pain, nociceptive pain, inflammatory pain, urinary incontinence, overactive bladder, emesis, cognitive disorders, anxiety, depression, sleeping disorders, eating disorders, movement disorders, glaucoma, psoriasis, multiple sclerosis, cerebrovascular disorders, brain injury, gastrointestinal disorders, hypertension, or cardiovascular disease.

This invention is also directed to pharmaceutical compositions comprising a therapeutically effective amount of a compound of Formula 1, an N-oxide or a pharmaceutically acceptable salt thereof for use in the manufacture of a medicament for the treatment of FAAH-mediated diseases, disorders or conditions including acute pain, chronic pain, neuropathic pain, nociceptive pain, inflammatory pain, urinary incontinence, overactive bladder, emesis, cognitive disorders, anxiety, depression, sleeping disorders, eating disorders, movement disorders, glaucoma, psoriasis, multiple sclerosis, cerebrovascular disorders, brain injury, gastrointestinal disorders, hypertension, or cardiovascular disease.

This invention relates to compounds of Formula 1 and pharmaceutically acceptable salts which are effective for inhibiting the activity of FAAH. Inhibition of FAAH activity can be measured by any method known in the art, for example, by measuring elevation in levels of fatty acid amides such as anandamide, oleamide, N-palmitoyl ehanolamide, and N-oleoyl ethanolamide. The invention also comprises pharmaceutical compositions comprising a therapeutically effective amount of a compound of Formula 1 or a pharmaceutically acceptable salt thereof and a pharmaceutically acceptable carrier. This invention is also directed to methods of treating FAAH-mediated diseases, disorders or conditions including acute pain, chronic pain, neuropathic pain, nociceptive pain, inflammatory pain, urinary incontinence, overactive bladder, emesis, cognitive disorders, anxiety, depression, sleeping disorders, eating disorders, movement disorders, glaucoma, psoriasis, multiple sclerosis, cerebrovascular disorders, brain injury, gastrointestinal disorders, hypertension, or cardiovascular disease in a subject by administering to a subject a therapeutically effective amount of one or more of the compounds of Formula 1 or a pharmaceutically acceptable salt thereof.

### DETAILS OF THE INVENTION

As used herein, the term “subject” refers to a mammal, including humans. The term “treating” refers to reversing, alleviating, inhibiting the progress of, or preventing a disease, disorder or condition to which such term applies, or to reversing, alleviating, inhibiting the progress of, or preventing one or more symptoms of such disease, disorder or condition. The phrase “therapeutically effective amount” refers to the quantity of a compound that may be used for treating a subject, which amount may depend on the weight and age of the subject and the route of administration, among other things. The terms “excipient” or “adjuvant” refer to any substance in a pharmaceutical formulation that is not an active pharmaceutical ingredient (API). The phrase “pharmaceutical composition” refers to the combination of one or more drug substances and one or more excipients. The phrases “drug product”, “pharmaceutical dosage form”, “dosage form”, “final dosage form” and the like, refer to a pharmaceutical composition that is administered to a subject in need of treatment and generally may be in the form of tablets, capsules, liquid solutions or suspensions, patches, films and the like.

Physiological pain is an important protective mechanism designed to warn of danger from potentially injurious stimuli from the external environment. The system operates through a specific set of primary sensory neurons and is activated by noxious stimuli via peripheral transducing mechanisms (see Millan, Prog. Neurobiol. 1999, 57, 1-164 for a review). These sensory fibers are known as nociceptors and are characteristically small diameter axons with slow conduction velocities. Nociceptors encode the intensity, duration and quality of noxious stimulus and by virtue of their topographically organized projection to the spinal cord, the location of the stimulus. The nociceptors are found on nociceptive nerve fibers of which there are two main types, A-delta fibers (myelinated) and C fibers (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g., painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitisation in the periphery, locally around the injury and centrally where the nociceptors terminate. These effects lead to a heightened sensation of pain. In acute pain these mechanisms can be useful, in promoting protective behaviors which may better enable repair processes to take place. Sensitivity is expected to return to normal once the injury has healed. However, in many chronic pain states, the hypersensitivity far outlasts the healing process and is often due to nervous system injury. This injury often leads to abnormalities in sensory nerve fibers associated with maladaptation and aberrant activity (Woolf & Salter Science 2000, 288, 1765-1768).

Clinical pain is present when discomfort and abnormal sensitivity feature among the patient's symptoms. Patients tend to be quite heterogeneous and may present with various pain symptoms. Such symptoms include: (1) spontaneous pain which may be dull, burning, or stabbing; (2) exaggerated pain responses to noxious stimuli (hyperalgesia); and (3) pain produced by normally innocuous stimuli (allodynia—Textbook of Pain Meyer et al. 1994, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Nociceptive pain is induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Textbook of Pain, Meyer et al, 1994, 13-44). The activation of nociceptors activates two types of afferent nerve fibers. Myelinated A-delta fibers transmit rapidly and are responsible for sharp and stabbing pain sensations, while unmyelinated C fibers transmit at a slower rate and convey a dull or aching pain. Moderate to severe acute nociceptive pain is a prominent feature of pain from central nervous system trauma, strains/sprains, burns, myocardial infarction and acute pancreatitis, postoperative pain (pain following any type of surgical procedure), posttraumatic pain, renal colic, cancer pain and back pain. Cancer pain may be chronic pain such as tumor related pain (e.g., bone pain, headache, facial pain or visceral pain) or pain associated with cancer therapy (e.g., postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain is currently defined as pain initiated or caused by a primary lesion or dysfunction in the nervous system. Nerve damage can be caused by trauma and disease and thus the term “neuropathic pain” encompasses many disorders with diverse etiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion Lancet 1999, 353, 1959-1964). The symptoms of neuropathic pain are difficult to treat, as they are often heterogeneous even between patients with the same disease (Woolf & Decosterd Pain Supp. 1999, 6, S141-S147; Woolf and Mannion Lancet 1999, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Textbook of Pain Levine and Taiwo, 1994, 45-56). Arthritic pain is the most common inflammatory pain. Rheumatoid disease is one of the commonest chronic inflammatory conditions in developed countries and rheumatoid arthritis is a common cause of disability. The exact etiology of rheumatoid arthritis is unknown, but current hypotheses suggest that both genetic and microbiological factors may be important (Textbook of Pain Grennan & Jayson, 1994, 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder Ann Pharmacother. 2002, 36, 679-686; Textbook of Pain McCarthy et al, 1994, 387-395). Most patients with osteoarthritis seek medical attention because of the associated pain. Arthritis has a significant impact on psychosocial and physical function and is known to be the leading cause of disability in later life. Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs.

Another type of inflammatory pain is visceral pain which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Other types of visceral pain include the pain associated with dysmenorrhea, cystitis and pancreatitis and pelvic pain.

It should be noted that some types of pain have multiple etiologies and thus can be classified in more than one area, e.g., back pain and cancer pain have both nociceptive and neuropathic components. Other types of pain include pain resulting from musculo-skeletal disorders, including myalgia, fibromyalgia, spondylitis, sero-negative (non-rheumatoid) arthropathies, non-articular rheumatism, dystrophinopathy, glycogenolysis, polymyositis and pyomyositis; heart and vascular pain, including pain caused by angina, myocardical infarction, mitral stenosis, pericarditis, Raynaud's phenomenon, scleredoma and skeletal muscle ischemia; head pain, such as migraine (including migraine with aura and migraine without aura), cluster headache, tension-type headache mixed headache and headache associated with vascular disorders; and orofacial pain, including dental pain, otic pain, burning mouth syndrome and temporomandibular myofascial pain.

As described above, the compounds herein, and the pharmaceutically acceptable salts thereof, can be used to treat CNS disorders, including schizophrenia and other psychotic disorders, mood disorders, anxiety disorders, sleep disorders, and cognitive disorders, such as delirium, dementia, and amnestic disorders. The standards for diagnosis of these disorders can be found in the American Psychiatric Association's Diagnostic and Statistical Manual of Mental Disorders (4th ed., 2000), which is commonly referred to as the DSM Manual.

For the purposes of this disclosure, schizophrenia and other psychotic disorders include schizophreniform disorder, schizoaffective disorder, delusional disorder, brief psychotic disorder, shared psychotic disorder, psychotic disorder due to general medical condition, and substance-induced psychotic disorder, as well as medication-induced movement disorders, such as neuroleptic-induced Parkinsonism, neuroleptic malignant syndrome, neuroleptic-induced acute dystonia, neuroleptic-induced acute akathisia, neuroleptic-induced tardive dyskinesia, and medication-induced postural tremor.

Mood disorders include depressive disorders, such as major depressive disorder, dysthymic disorder, premenstrual dysphoric disorder, minor depressive disorder, recurrent brief depressive disorder, postpsychotic depressive disorder of schizophrenia, and major depressive episode with schizophrenia; bipolar disorders, such as bipolar I disorder, bipolar II disorder, cyclothymia, and bipolar disorder with schizophrenia; mood disorders due to general medical condition; and substance-induced mood disorders.

Anxiety disorders include panic attack, agoraphobia, panic disorder without agoraphobia, agoraphobia without history of panic disorder, specific phobia, social phobia (social anxiety disorder), obsessive-compulsive disorder, posttraumatic stress disorder, acute stress disorder, generalized anxiety disorder, anxiety disorder due to general medical condition, substance-induced anxiety disorder, and mixed anxiety-depressive disorder.

Sleep disorders include primary sleep disorders, such as dyssomnias (primary insomnia, primary hypersomnia, narcolepsy, breathing-related sleep disorder, circadian rhythm sleep disorder, sleep deprivation, restless legs syndrome, and periodic limb movements) and parasomnias (nightmare disorder, sleep terror disorder, sleepwalking disorder, rapid eye movement sleep behavior disorder, and sleep paralysis); sleep disorders related to another mental disorder, including insomnia related to schizophrenia, depressive disorders, or anxiety disorders, or hypersomnia associated with bipolar disorders; sleep disorders due to a general medical condition; and substance-induced sleep disorders. Delirium, dementia, and amnestic and other cognitive disorders, includes delirium due to a general medical condition, substance-induced delirium, and delirium due to multiple etiologies; dementia of the Alzheimer's type, vascular dementia, dementia due to general medical conditions, dementia due to human immunodeficiency virus disease, dementia due to head trauma, dementia due to Parkinson's disease, dementia due to Huntington's disease, dementia due to Pick's disease, dementia due to Creutzfeldt-Jakob disease, dementia due to other general medical conditions, substance-induced persisting dementia, dementia due to multiple etiologies; amnestic disorders due to a general medical condition, and substance-induced persisting amnestic disorder.

Substance-induced disorders refer to those resulting from the using, abusing, dependence on, or withdrawal from, one or more drugs or toxins, including alcohol, amphetamines or similarly acting sympathomimetics, caffeine, cannabis, cocaine, hallucinogens, inhalants, nicotine, opioids, phencyclidine or similarly acting arylcyclohexylamines, and sedatives, hypnotics, or anxiolytics, among others.

Urinary incontinence includes the involuntary or accidental loss of urine due to the inability to restrain or control urinary voiding. Urinary incontinence includes mixed urinary incontinence, nocturnal enuresis, overflow incontinence, stress incontinence, transient urinary incontinence, and urge incontinence.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

In the above recitations, the term “alkyl”, used either alone or in compound words such as “alkylthio” or “haloalkyl” includes straight-chain or branched alkyl, such as, methyl, ethyl, n-propyl, i-propyl, and the different butyl isomers. “Alkenyl” includes straight-chain or branched alkenes such as ethenyl, 1-propenyl, 2-propenyl, and the different butenyl isomers. “Alkenyl” also includes polyenes such as 1,2-propadienyl. “Alkynyl” includes straight-chain or branched alkynes such as ethynyl, 1-propynyl, 2-propynyl, and the different butynyl isomers.

“Alkoxy” includes, for example, methoxy, ethoxy, n-propyloxy, i-propyloxy, and the different butoxy isomers. “Alkylthio” includes branched or straight-chain alkylthio moieties such as methylthio, ethylthio, and the different propylthio and butylthio isomers. “Alkylsulfinyl” includes both enantiomers of an alkylsulfinyl group. Examples of “alkylsulfinyl” include CH₃S(═O), CH₃CH₂S(═O), CH₃CH₂CH₂S(═O), (CH₃)₂CHS(═O), and the different butylsulfinyl isomers. Examples of “alkylsulfonyl” include CH₃S(═O)₂, CH₃CH₂S(═O)₂, CH₃CH₂CH₂S(═O)₂, (CH₃)₂CHS(═O)₂, and the different butylsulfonyl isomers. “Alkylamino” includes an NH radical substituted with straight-chain or branched alkyl. Examples of “alkylamino” include CH₃CH₂NH, CH₃CH₂CH₂NH and (CH₃)₂CHCH₂NH. Examples of “dialkylamino” include (CH₃)₂N, (CH₃CH₂CH₂)₂N and CH₃CH₂(CH₃)N. “Alkylcarbonyl” denotes a straight-chain or branched alkyl bonded to a C(═O) moiety. Examples of “alkylcarbonyl” include CH₃C(═O), CH₃CH₂CH₂C(═O) and (CH₃)₂CHC(═O).

“Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkoxycarbonyl” denotes alkyloxy substitution bonded to a C(═O) moiety. Examples of “alkoxycarbonyl” include CH₃C(═O), CH₃CH₂OC(═O), CH₃CH₂CH₂C(═O), (CH₃)₂CHOC(═O), and the different butoxy-, pentoxy- or hexoxycarbonyl isomers. The term “alkylcarbonyloxy” denotes straight-chain or branched alkylcarbonyl attached to and linked through an oxygen atom. Examples of “alkylcarbonyloxy” include CH₃CH₂C(═O)O and (CH₃)₂CHC(═O)O.

“Alkoxyalkoxyalkyl” denotes alkoxy substitution on alkoxyalkyl. Examples of “alkoxyalkoxyalkyl” include CH₃OCH₂OCH₂, CH₃OCH₂OCH₂CH₂, CH₃CH₂OCH₂OCH₂ and CH₃OCH₃CH₂OCH₂CH₂.

“Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂ and CH₃CH₂SCH₂CH₂; “alkylsulfinylalkyl” and “alkylsulfonylalkyl” include the corresponding sulfoxides and sulfones, respectively. “Alkylcarbonylthio” denotes straight-chain or branched alkylcarbonyl attached to and linked through a sulfur atom. Examples of “alkylcarbonylthio” include CH₃C(═O)S, CH₃CH₂CH₂C(═O)S and (CH₃)₂CHC(═O)S.

“Alkylaminoalkyl” denotes alkylamino substitution on alkyl. Examples of “alkylaminoalkyl” include CH₃NHCH₂, CH₃NHCH₂CH₂, CH₃CH₂NHCH₂, CH₃CH₂CH₂CH₂NHCH₂ and CH₃CH₂NHCH₂CH₂. Examples of “dialkylaminoalkyl” include ((CH₃)₂CH)₂NCH₂, (CH₃CH₂CH₂)₂NCH₂ and CH₃CH₂(CH₃)NCH₂CH₂. The term “alkylaminocarbonyl” denotes straight-chain or branched alkylamino bonded to a C(═O) moiety. Examples of “alkylaminocarbonyl” include CH₃NHC(═O), CH₃CH₂NHC(═O), CH₃CH₂CH₂NHC(═O), (CH₃)₂CHNHC(═O) and the different butylamino- or pentylaminocarbonyl isomers. Examples of “dialkylaminocarbonyl” include (CH₃)₂NC(═O), (CH₃CH₂)₂NC(═O), CH₃CH₂(CH₃)NC(═O), (CH₃)₂CH(CH₃)NC(═O) and CH₃CH₂CH₂(CH₃)NC(═O).

“Hydroxyalkyl” denotes an alkyl group substituted with one hydroxy group. Examples of “hydroxyalkyl” include HOCH₂CH₂, CH₃CH₂(OH)CH and HOCH₂CH₂CH₂CH₂.

The term “cycloalkyl” denotes a saturated carbocyclic ring consisting of 3 to 8 carbon atoms linked to one another by single bonds. Examples of “cycloalkyl” include cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, methylcyclopentyl and methylcyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. The term “cycloalkoxyalkyl” denotes cycloalkoxy substitution on an alkyl moiety. Examples of “cycloalkoxyalkyl” include cyclopropyloxymethyl, cyclopentyloxyethyl, and other cycloalkoxy moieties bonded to straight-chain or branched alkyl groups. The term “cycloalkylaminoalkyl” denotes cycloalkylamino substitution on an alkyl group. Examples of “cycloalkylaminoalkyl” include cyclopropylaminomethyl, cyclopentylaminoethyl, and other cycloalkylamino moieties bonded to straight-chain or branched alkyl groups.

“Trialkylsilyl” includes 3 branched and/or straight-chain alkyl radicals attached to and linked through a silicon atom, such as trimethylsilyl, triethylsilyl and tert-butyldimethylsilyl.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The terms “haloalkenyl”, “haloalkynyl”, “haloalkoxy”, “haloalkylthio”, “haloalkylsulfinyl”, “haloalkylsulfonyl”, “halocycloalkyl”, and the like, are defined analogously to the term “haloalkyl”. Examples of “haloalkenyl” include Cl₂C═CHCH₂ and CF₃CH₂CH═CH. Examples of “haloalkynyl” include HCCCHCl, CF₃C≡C, CCl₃C≡C and FCH₂CCCH₂. Examples of “haloalkoxy” include CF₃O, CCl₃CH₂O, F₂CHCH₂CH₂O and CF₃CH₂O. Examples of “haloalkylthio” include CCl₃S, CF₃S, CCl₃CH₂S and ClCH₂CH₂CH₂S. Examples of “haloalkylsulfinyl” include CF₃S(═O), CCl₃S(═O), CF₃CH₂S(═O) and CF₃CF₂S(═O). Examples of “haloalkylsulfonyl” include CF₃S(═O)₂, CCl₃S(═O)₂, CF₃CH₂S(═O)₂ and CF₃CF₂S(═O)₂. Examples of “halocycloalkyl” include chlorocyclopropyl, fluorocyclobutyl and chlorocyclohexyl.

The total number of carbon atoms in a substituent group is indicated by the “Cᵢ-Cⱼ” prefix where i and j are numbers from 1 to 10. For example, C₁-C₄ alkylsulfonyl designates methylsulfonyl through butylsulfonyl; C₂ alkoxyalkyl designates CH₃OCH₂; C₃ alkoxyalkyl designates, for example, CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

The term “unsubstituted” in connection with a group such as a ring means the group does not have any substituents other than its one or more attachments to the remainder of Formula 1. The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) range from 1 to 3. As used herein, the term “optionally substituted” is used interchangeably with the phrase “substituted or unsubstituted” or with the term “(un)substituted.”

The number of optional substituents may be restricted by an expressed limitation. For example, the phrase “optionally substituted with up to 2 substituents independently selected from R^{9a} on carbon atom ring members” means that 0, 1 or 2 substituents can be present (if the number of potential connection points allows). Similarly, the phrase “optionally substituted with up to 3 substituents independently selected from R^{5a} on carbon atom ring members” means that 0, 1, 2 or 3 substituents can be present if the number of available connection points allows. When a range specified for the number of substituents (e.g., k being an integer from 0 to 3 in Exhibit 1) exceeds the number of positions available for substituents on a ring (e.g., only 2 positions are available for (R^{v})ₖ on U-12 in Exhibit 1), the actual higher end of the range is recognized to be the number of available positions.

When a group is substituted with a substituent bearing a subscript that indicates the number of said substituents can exceed 1, said substituents (when they exceed 1) are independently selected from the group of defined substituents (e.g., (R^{v})ₖ wherein k is 1, 2, or 3 in Exhibit 1). When a group is substituted with a substituent bearing a subscript that indicates the substituent to be optionally attached, for example (R³)ₘ wherein m can be zero, then hydrogen may be at the position regardless of wherther hydrogen is recited in the variable group definition. When a group contains a substituent which can be hydrogen, for example R^{2a}, then when this substituent is taken as hydrogen, it is recognized that this is equivalent to said group being unsubstituted. When one or more positions on a group are said to be “not substituted” or “unsubstituted”, then hydrogen atoms are attached to take up any free valency.

The term “ring member” refers to an atom (e.g., C, O, N or S) or other moiety (e.g., C(═O), C(═S) or)S(═O)ᵤ(═NR¹⁰)_{z}) forming the backbone of a ring or ring system.

“Aromatic” indicates that each of the ring atoms is essentially in the same plane and has a p-orbital perpendicular to the ring plane, and that (4n+2) π electrons, where n is a positive integer, are associated with the ring to comply with Mickel's rule. An aromatic ring system denotes a carbocyclic or heterocyclic ring system in which at least one ring of the ring system is aromatic. An aromatic heterocyclic ring system denotes a heterocyclic ring system in which at least one ring of the ring system is aromatic.

The term “carbocyclic ring” denotes a ring wherein the atoms forming the ring backbone are selected only from carbon. Unless otherwise indicated, a carbocyclic ring can be a saturated, partially unsaturated, or fully unsaturated ring. When a fully unsaturated carbocyclic ring satisfies Mickel's rule, then said ring is also called an “aromatic ring”. “Saturated carbocyclic” refers to a ring having a backbone consisting of carbon atoms linked to one another by single bonds; unless otherwise specified, the remaining carbon valences are occupied by hydrogen atoms.

The terms “heterocyclic ring”, “heterocycle” or “heterocyclic ring system” denote a ring or ring system in which at least one atom forming the ring backbone is not carbon, e.g., nitrogen, oxygen or sulfur. Typically a heterocyclic ring contains no more than 2 nitrogens, no more than 2 oxygens and no more than 2 sulfurs. Unless otherwise indicated, a heterocyclic ring can be a saturated, partially unsaturated, or fully unsaturated ring. When a fully unsaturated heterocyclic ring satisfies Mickel's rule, then said ring is also called a “heteroaromatic ring” or “aromatic heterocyclic ring”. Unless otherwise indicated, heterocyclic rings and ring systems can be attached through any available carbon or nitrogen by replacement of a hydrogen on said carbon or nitrogen.

As noted in the Summary of the Invention, a pair of R^{8a} and R³ substituents besides the possibility of being separate substituents, may also be connected to form a ring. The portion of the ring form by joining R^{8a} and R³ can contain 5-, 6- or 7-members including as ring members the two carbon atoms to which the substituents R^{8a} and R³ are attached. The other 3 to 5 ring members are provided by the pair of R^{5a} and R³ substituents taken together.

These other ring members are selected from carbon atoms and up to 2 heteroatoms independently selected from up to 1 O, up to 1S, up to 1 N, wherein up to 2 carbon atom ring members are independently selected from C(═O) and C(═S), the sulfur atom ring member is selected from)S(═O)ᵤ(═NR¹⁰)_{z}, each ring optionally substituted with up to 2 substituents independently selected from R⁹ on carbon atom ring members and R^{9b} on the nitrogen atom ring member. In this definition the heteroatoms are optional, because the number of heteroatom ring members may be zero. The nitrogen atom ring members may be oxidized as N-oxides, because compounds relating to Formula 1 also include N-oxide derivatives. The portion of the ring system formed by the pair of R^{8a} and R³ taken together can be optionally substituted with up to 2 substituents independently selected from R^{9a} on carbon atom ring members and R^{9b} on the nitrogen atom ring member.

As noted in the Summary of the Invention, a pair of R^{8b} and R³ substituents besides the possibility of being separate substituents, may also be connected to form a ring. The portion of the ring taken form by joining R^{8b} and R³ can contain 5-, 6- or 7-members including as ring members the carbon and nitrogen atoms to which the substituents R^{8b} and R³ are attached. The other 3 to 5 ring members are provided by the pair of R^{8b} and R³ substituents taken together. These other ring members are selected from carbon atoms and 1 to 2 heteroatoms independently selected from up to 1 O, up to 1S, up to 1 N, wherein up to 2 carbon atom ring members are independently selected from C(═O) and C(═S), the sulfur atom ring member is selected from)S(═O)ᵤ(═NR¹⁰)_{z}, each ring optionally substituted with up to 2 substituents independently selected from R⁹ on carbon atom ring members and R^{9b} on the nitrogen atom ring member. In this definition the nitrogen atom ring members may be oxidized as N-oxides, because compounds relating to Formula 1 also include N-oxide derivatives. The portion of the ring system formed by the pair of R^{8b} and R³ taken together can be optionally substituted with up to 2 substituents independently selected from R^{9a} on carbon atom ring members and R^{9b} on the nitrogen atom ring member.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form. For example, Formula 1 possesses a chiral center at the carbon atom to which R⁴ is bonded. The two enantiomers are depicted as Formula 1′ and Formula 1″ with the chiral center identified with an asterisk (*).

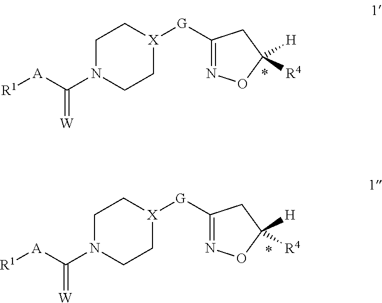

Compounds of Formula 1 comprise racemic mixtures, for example, equal amounts of the enantiomers of Formulae 1′ and 1″. In addition, compounds of Formula 1 include compounds that are enriched compared to the racemic mixture in an enantiomer of Formula 1. Also included are the essentially pure enantiomers of compounds of Formula 1, for example, Formula 1′ and Formula 1″.

Compounds of Formula 1 can comprise additional chiral centers. For example, substituents and other molecular constituents such as R² and R³ may themselves contain chiral centers. This invention comprises racemic mixtures as well as enriched and essentially pure stereoconfigurations at these additional chiral centers.

Molecular depictions drawn herein follow standard conventions for depicting stereochemistry. To indicate stereoconfiguration, bonds rising from the plane of the drawing and towards the viewer are denoted by solid wedges wherein the broad end of the wedge is attached to the atom rising from the plane of the drawing towards the viewer. Bonds going below the plane of the drawing and away from the viewer are denoted by dashed wedges wherein the narrow end of the wedge is attached to the atom further away from the viewer. Constant width lines indicate bonds with a direction opposite or neutral relative to bonds shown with solid or dashed wedges; constant width lines also depict bonds in molecules or parts of molecules in which no particular stereoconfiguration is intended to be specified.

When enantiomerically enriched, one enantiomer is present in greater amounts than the other, and the extent of enrichment can be defined by an expression of enantiomeric excess (“ee”), which is defined as (2x−1)·100%, where x is the mole fraction of the dominant enantiomer in the mixture (e.g., an ee of 20% corresponds to a 60:40 ratio of enantiomers).

Preferably the compositions of this invention of Formula 1 have at least a 50% enantiomeric excess; more preferably at least a 75% enantiomeric excess; still more preferably at least a 90% enantiomeric excess; and the most preferably at least a 94% enantiomeric excess of the more active isomer. Of particular note are enantiomerically pure embodiments of the more active isomer.

Compounds of Formula 1 can exist as one or more conformational isomers due to restricted rotation about the amide bond (e.g., C(═W)—N) in Formula 1. Compounds of Formula 1 comprise mixtures of conformational isomers. In addition, compounds of Formula 1 include compounds that are enriched in one conformer relative to others.

The compounds of the present invention include N-oxide derivatives of Formula 1. One skilled in the art will appreciate that not all nitrogen-containing heterocycles can form N-oxides since the nitrogen requires an available lone pair of electrons for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as tert-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. When the compounds forming the present mixtures and compositions contain acidic or basic moieties, a wide variety of salts can be formed, and these salts are useful in the present mixtures and compositions for controlling plant diseases caused by fungal plant pathogens (i.e. are agriculturally suitable). When a compound contains a basic moiety such as an amine function, salts include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound contains an acidic moiety such as a carboxylic acid or phenol, salts include those formed with organic or inorganic bases such as pyridine, triethylamine or ammonia, or amides, hydrides, hydroxides or carbonates of sodium, potassium, lithium, calcium, magnesium or barium.

The compounds described and specifically named herein may form pharmaceutically acceptable complexes, salts, solvates and hydrates. The salts include acid addition salts and base salts.

Pharmaceutically acceptable acid addition salts include salts derived from inorganic acids such as hydrochloric acid, nitric acid, phosphoric acid, sulfuric acid, hydrobromic acid, hydroiodic acid, hydrofluoric acid, and phosphorous acids, as well salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts include acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride, chloride, hydrobromide, bromide, hydroiodide, iodide, isothionate, lactate, malate, maleate, malonate, mesylate, methylsulfate, naphthylate, 2-napsylate, nicotinate, nitrate, orotate, oxalate, almitate, pamoate, phosphate, hydrogen phosphate, dihydrogen phosphate, pyroglutamate, saccharate, stearate, succinate, tannate, tartrate, tosylate, trifluoroacetate and xinofoate salts.

Pharmaceutically acceptable base salts include salts derived from bases, including metal cations, such as an alkali or alkaline earth metal cation, as well as amines. Examples of suitable metal cations include sodium (Na⁺), potassium (K⁺), magnesium (Mg²⁺), calcium (Ca²⁺), zinc (Zn²⁺), and aluminum (Al³⁺). Examples of suitable amines include arginine, N,N′-dibenzylethylenediamine, chloroprocaine, choline, diethylamine, diethanolamine, dicyclohexylamine, ethylenediamine, glycine, lysine, N-methylglucamine, olamine, 2-amino-2-hydroxymethyl-propane-1,3-diol, and procaine. For a discussion of useful acid addition and base salts, see S. M. Berge et al., “Pharmaceutical Salts,” J. Pharm. Sci., 1977, 66, 1-19; see also Stahl and Wermuth, Handbook of Pharmaceutical Salts: Properties, Selection, and Use (2002).

The compounds herein, and the pharmaceutically acceptable salts thereof, may exist in a continuum of solid states ranging from fully amorphous to fully crystalline. They may also exist in unsolvated and solvated forms. The term “solvate” describes a molecular complex comprising the compound and one or more pharmaceutically acceptable solvent molecules (e.g., EtOH). The term “hydrate” is a solvate in which the solvent is water. Pharmaceutically acceptable solvates include those in which the solvent may be isotopically substituted (e.g., D₂O, d₆-acetone, d₆-DMSO).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will be the norm.

Compounds selected from Formula 1, stereoisomers, N-oxides, and salts thereof, typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.

The compounds herein, and the pharmaceutically acceptable salts thereof, may also exist as multicomponent complexes (other than salts and solvates) in which the compound and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together. See, e.g., O. Almarsson and M. J. Zaworotko, Chem. Commun. 2004 17, 1889-1896. For a general review of multi-component complexes, see J. K. Haleblian, J. Pharm. Sci. 1975, 64, 1269-88.

The invention includes prodrugs and metabolites of the compounds of Formula 1. “Prodrugs” refer to compounds that when metabolized in vivo, undergo conversion to compounds having the desired pharmacological activity. Prodrugs may be prepared by replacing appropriate functionalities present in pharmacologically active compounds with “pro-moieties” as described, for example, in H. Bundgaar, Design of Prodrugs (1985). Examples of prodrugs include ester, ether or amide derivatives of the compounds herein, and their pharmaceutically acceptable salts. For further discussions of prodrugs, see e.g., T. Higuchi and V. Stella “Pro-drugs as Novel Delivery Systems,” ACS Symposium Series 14 (1975) and E. B. Roche ed., Bioreversible Carriers in Drug Design (1987).

“Metabolites” refer to compounds formed in vivo upon administration of pharmacologically active compounds. Examples include hydroxymethyl, hydroxy, secondary amino, primary amino, phenol, and carboxylic acid derivatives of compounds herein, and the pharmaceutically acceptable salts thereof having methyl, alkoxy, tertiary amino, secondary amino, phenyl, and amide groups, respectively.

Compounds described herein also include all pharmaceutically acceptable isotopic variations, in which at least one atom is replaced by an atom having the same atomic number, but an atomic mass different from the atomic mass usually found in nature. Isotopes suitable for inclusion in the compounds herein, and the pharmaceutically acceptable salts thereof include, for example, isotopes of hydrogen, such as ²H and ³H; isotopes of carbon, such as ¹¹C, ¹³C and ¹⁴C; isotopes of nitrogen, such as ¹³N and ¹⁵N; isotopes of oxygen, such as ¹⁵O, ¹⁷O and ¹⁸O; isotopes of sulfur, such as ³⁵S; isotopes of fluorine, such as ¹⁸F; isotopes of chlorine, such as ³⁶Cl, and isotopes of iodine, such as ¹²³I and ¹²⁵I. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies. Substitution with positron emitting isotopes, such as ¹¹C, ¹⁸F, ¹⁵O and ¹³N, may be useful in Positron Emission Topography (PET) studies for examining substrate receptor occupancy. Isotopically-labelled compounds may be prepared by processes analogous to those described elsewhere in the disclosure using an appropriate isotopically-labelled reagent in place of a non-labelled reagent.

Embodiments of the present invention as described in the Summary of the Invention include those described below. In the following Embodiments, Formula 1 includes N-oxides and salts thereof, and reference to “a compound of Formula 1” includes the definitions of substituents specified in the Summary of the Invention unless further defined in the Embodiments.
Embodiment 1. The method described in the Summary of the Invention for treating a subject suffering from or diagnosed with a disease, disorder, or condition mediated by fatty acid amide hydrolase activity, said method comprising administering to the subject in need of such treatment an effective amount of a compound selected from compounds of Formula 1.
Embodiment 2. The method of Embodiment 1 wherein A is O or S.
Embodiment 3. The method of Embodiment 1 wherein A is O or NR⁶.
Embodiment 3a. The method of Embodiment 3 wherein R⁶ is H, C₁-C₄ alkyl, C₂-C₄ alkenyl, C₂-C₄ alkynyl, C₁-C₄ haloalkyl, C₂-C₄ haloalkenyl or C₂-C₄ haloalkynyl.
Embodiment 4. The method of Embodiment 3a wherein R⁶ is H.
Embodiment 5. The method of any one of Embodiments 1 through 4 wherein A is O or NH.
Embodiment 6. The method of Embodiment 5 wherein A is O.
Embodiment 7. The method of Embodiment 5 wherein A is NH.
Embodiment 8. The method of any one of Embodiments 1 through 7 wherein W is O.
Embodiment 9. The method of any one of Embodiments 1 through 8 wherein X is CR^{2a} or N.

Embodiment 10. The method of Embodiment 9 wherein X is CR^{2a}.
Embodiment 10a. The method of Embodiment 9 wherein R^{2a} is H.
Embodiment 11. The method of Embodiment 9 wherein X is N.
Embodiment 12. The method of any one of Embodiments 1 through 11 wherein R¹ is selected from U-1 through U-51 as shown in Exhibit 1

### Exhibit 1

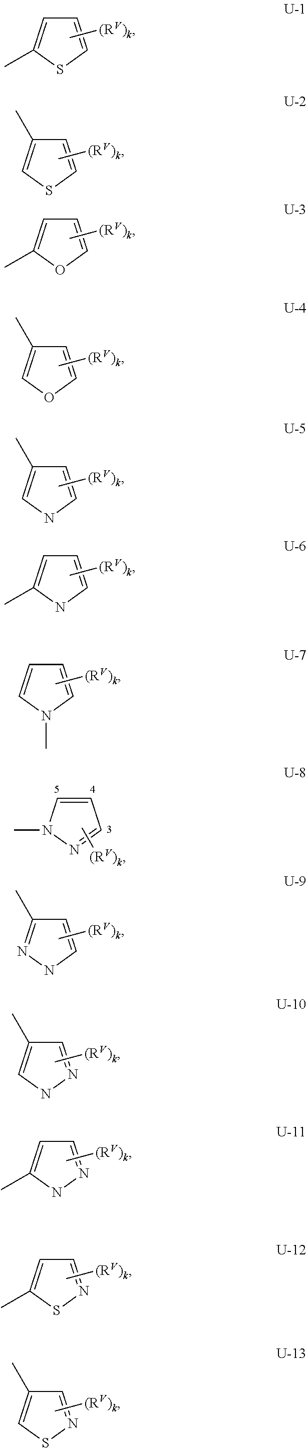 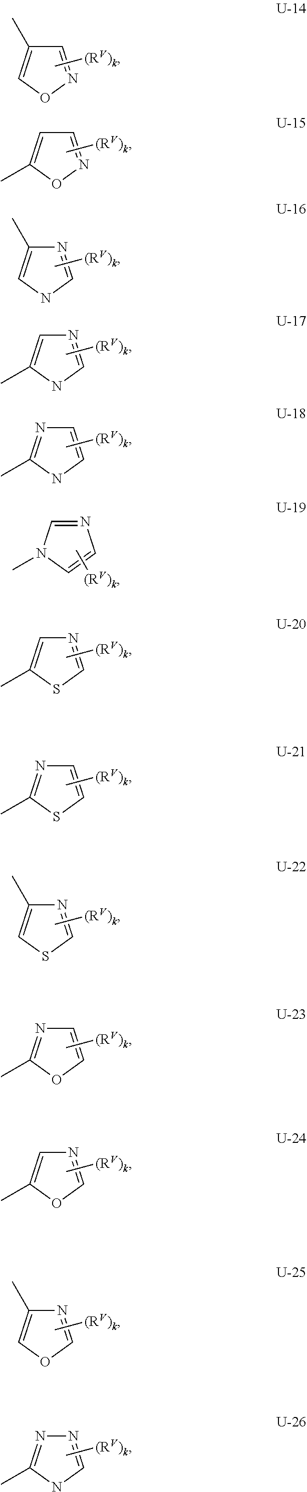 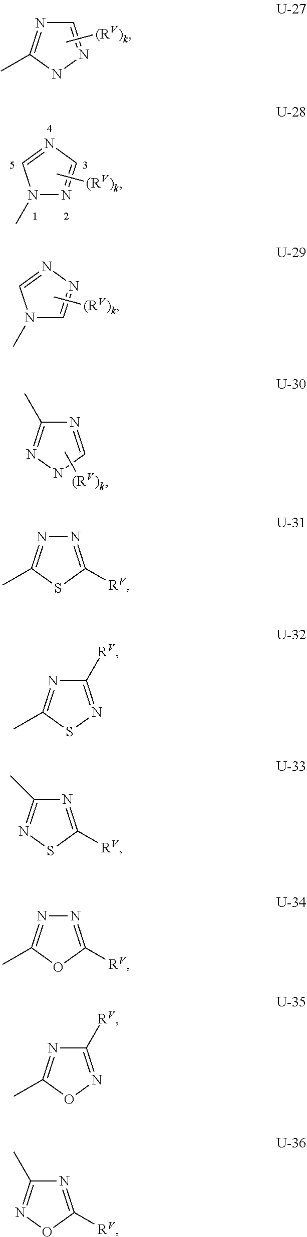 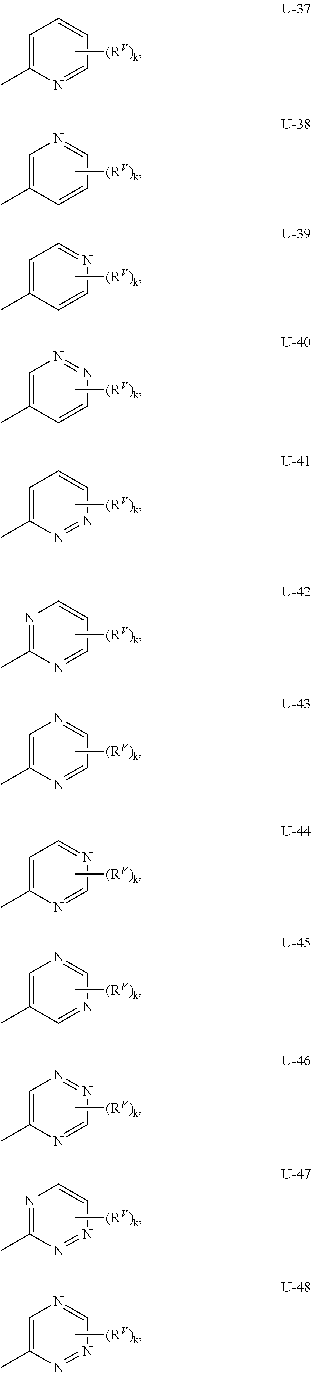 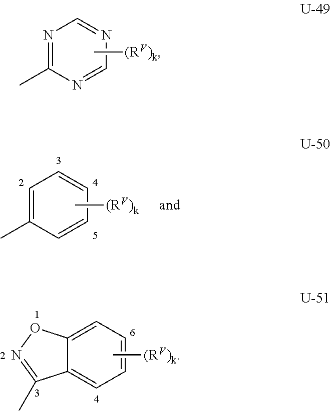 [0117] wherein each R^{V} is independently selected from H and R^{5a} when R^{V} is attached to a carbon atom ring member, and R^{V} is selected from H and R^{5b} when R^{V} is attached to a nitrogen atom ring member (e.g., U-5, U-6, U-9, U-10, U-11, U-16, U-17, U-18, U-26, U-27 or U-30), and the bond projecting to the left is bonded to A of Formula 1; k is 0, 1, 2 or 3.
Embodiment 13. The method of any one of Embodiments 1 through 12 wherein each R^{5a} is independently halogen, hydroxy, cyano, nitro, C₁-C₄ alkyl, C₁-C₆ haloalkyl, C₁-C₄ alkoxy, C₁-C₄ haloalkoxy, C₁-C₄ alkylthio, C₁-C₄ haloalkylthio, C₁-C₄ alkylsulfinyl, C₁-C₄ alkylsulfonyl, C₁-C₄ haloalkylsulfinyl, C₁-C₄ haloalkylsulfonyl, C₂-C₈ dialkylamino, C₂-C₄ alkylcarbonyl, C₂-C₆ alkoxycarbonyl or C₂-C₆ alkylcarbonyloxy.
Embodiment 14. The method of Embodiment 13 wherein each R^{5a} is independently halogen, cyano, nitro, C₁-C₂ alkyl, C₁-C₂ haloalkyl, C₁-C₂ alkoxy or C₁-C₂ haloalkoxy.
Embodiment 15. The method of Embodiment 14 wherein each R^{5a} is independently halogen, nitro, C₁-C₂ alkyl, C₁-C₂ haloalkyl or C₁-C₂ alkoxy.
Embodiment 16. The method of Embodiment 15 wherein each R^{5a} is independently bromo, chloro, methyl, trifluoromethyl or methoxy.
Embodiment 17. The method of Embodiment 16 wherein each R^{5a} is independently chloro, methyl, trifluoromethyl or methoxy.
Embodiment 18. The method of any one of Embodiments 1 through 17 wherein each R^{5b} is independently C₁-C₄ alkyl, C₁-C₄ haloalkyl or C₂-C₄ alkoxyalkyl.
Embodiment 19. The method of Embodiment 18 wherein each R^{5b} is independently C₁-C₄ alkyl.
Embodiment 20. The method of Embodiment 20 wherein each R^{5b} is methyl.
Embodiment 21. The method of Embodiment 12 wherein R^{V} is H.
Embodiment 22. The method of Embodiments 12 wherein k is 0.
Embodiment 23. The method of any one of Embodiments 12 through 22 wherein R¹ is selected from U-21 and U-37 through U-51.
Embodiment 24. The method of Embodiment 23 wherein R¹ is selected from U-21, U-37, U-38, U-39, U-42, U-44, U-50 and U-51.
Embodiment 25. The method of Embodiment 24 wherein R¹ is selected from U-21, U-50 and U-51.
Embodiment 26. The method of any one of Embodiments 1 through 25 wherein each R² is independently C₁-C₂ alkyl or C₁-C₂ haloalkyl.
Embodiment 27. The method of any one of Embodiments 1 through 26 wherein n is 0 or 1.

Embodiment 28. The method of Embodiment 27 wherein n is 0.
Embodiment 29. The method of any one of Embodiments 1 through 28 wherein each R³ when taken alone (i.e. not taken together with R^{8a} or R^{8b}) is independently cyano or C₁-C₃ alkyl.
Embodiment 30. The method of Embodiment 29 wherein each R³ when taken alone is independently cyano or C₁-C₂ alkyl.
Embodiment 31. The method of any one of Embodiments 1 through 31 wherein each R³ is taken alone (i.e. not taken together with R^{8a} or R^{8b}).
Embodiment 32. The method of any one of Embodiments 1 through 31 wherein m is 0 or 1.
Embodiment 33. The method of Embodiment 32 wherein m is 0.
Embodiment 34. The method of any one of Embodiments 1 through 33 wherein R⁴ is benzyl, phenyl or naphthalenyl, each optionally substituted with up to 3 substituents independently selected from R^{8a}; or pyridinyl, thienyl, pyrazolyl, triazolyl or imidazolyl, each optionally substituted with up to 3 substituents independently selected from R^{8a} on carbon atom ring members and R^{8b} on a nitrogen atom ring member.
Embodiment 35. The method of Embodiment 34 wherein R⁴ is benzyl or phenyl, each optionally substituted with up to 3 substituents independently selected from R^{8a}; or pyridinyl or thienyl, each optionally substituted with up to 3 substituents independently selected from R^{8a} on carbon atom ring members.
Embodiment 36. The method of Embodiment 35 wherein R⁴ is phenyl optionally substituted with up to 3 substituents independently selected from R.
Embodiment 37. The method of Embodiment 36 wherein R⁴ is phenyl optionally substituted with up to 2 substituents independently selected from R.
Embodiment 38. The method of Embodiment 37 wherein R⁴ is phenyl.
Embodiment 39. The method of any one of Embodiments 1 through 37 wherein each R^{8a} when taken alone (i.e., not taken together with R³) is independently halogen, hydroxy, amino, cyano, nitro, C₁-C₃ alkyl, C₁-C₃ haloalkyl, C₁-C₃ alkoxy, C₁-C₃ haloalkoxy, C₁-C₃ alkylthio or C₁-C₃ haloalkylthio.
Embodiment 40. The method of Embodiment 41 wherein each R^{8a} when taken alone is independently halogen, methyl, halomethyl or methoxy.
Embodiment 41. The method of any one of Embodiments 1 through 40 wherein each R^{8a} is taken alone (i.e., not taken together with R³).
Embodiment 42. The method of any one of Embodiments 1 through 34 wherein each R^{8b} when taken alone (i.e. not taken together with R³) is independently C₁-C₃ alkyl.
Embodiment 43. The method of Embodiment 42 wherein each R^{8b} when taken alone (i.e. not taken together with R³) is methyl.
Embodiment 44. The method of any one of Embodiments 1 through 43 wherein each R^{8b} is taken alone (i.e. not taken together with R³).
Embodiment 45. The method of any one of Embodiments 1 through 44 wherein G is selected from G-1 through G-48 as shown in Exhibit 2

### Exhibit 2

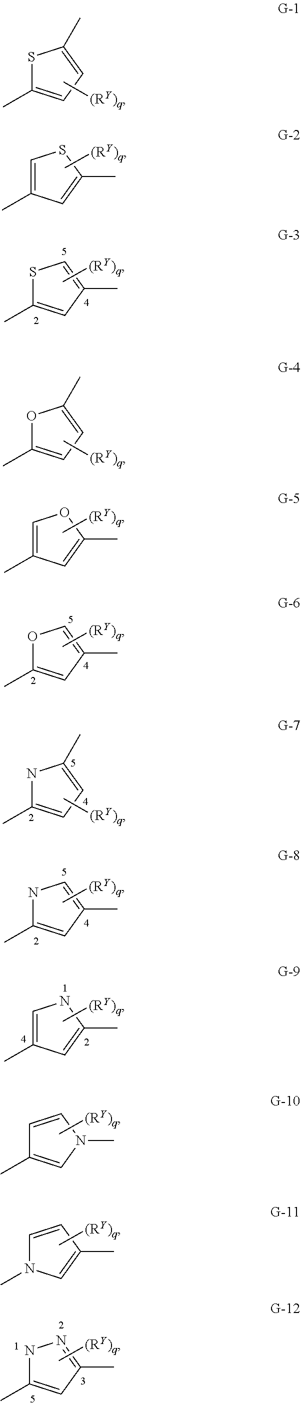 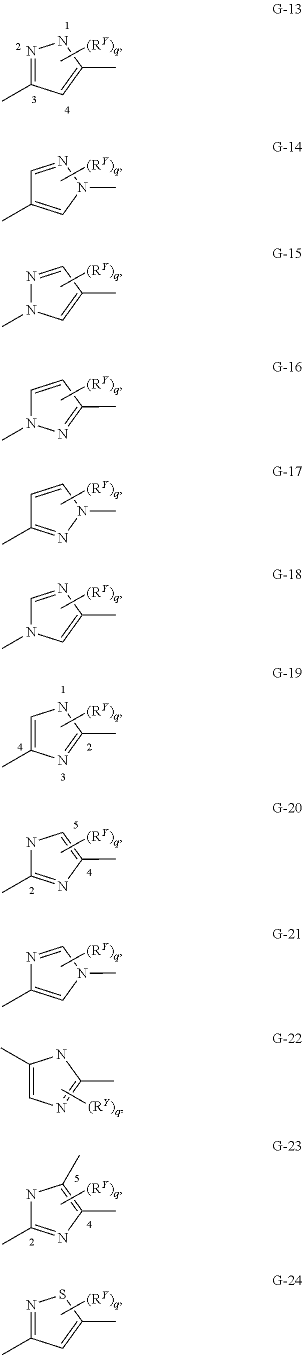 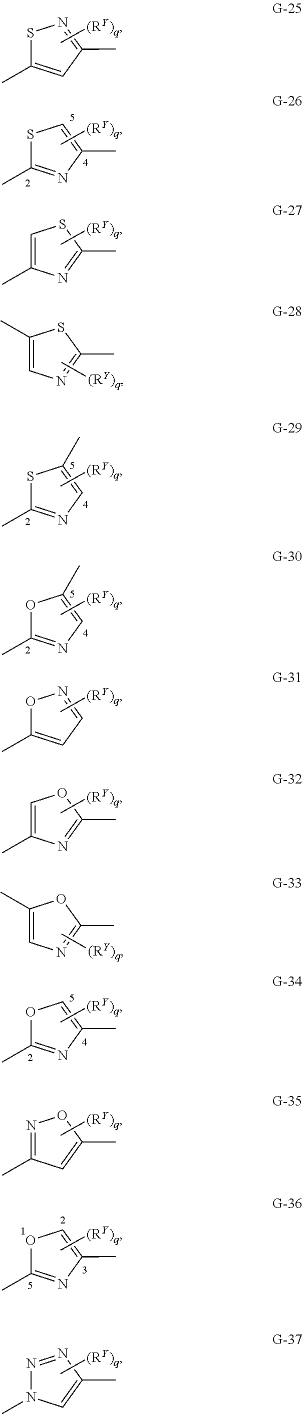 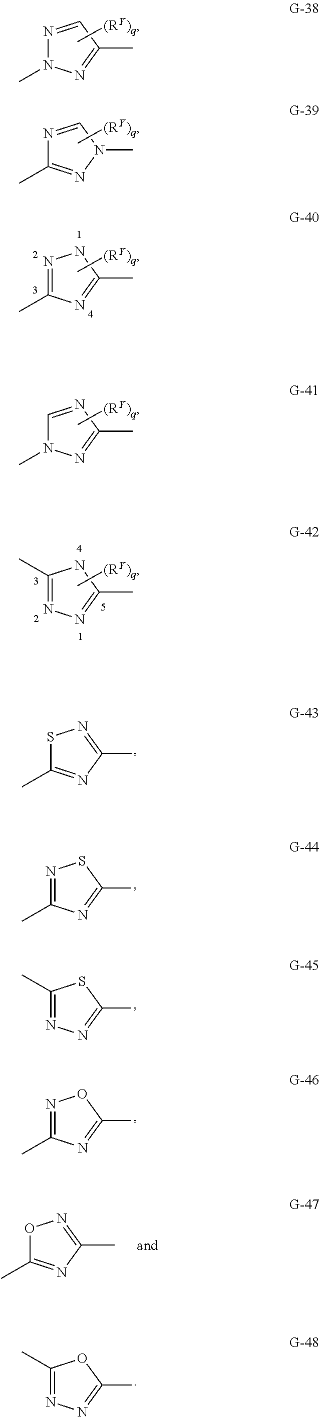 [0152] wherein R^{Y} is selected from H and R^{7a}, when R^{Y} is attached to a carbon atom ring member, and R^{Y} is selected from H and R^{7b} when R^{Y} is attached to a nitrogen atom ring member, and the bond projecting to the left is bonded to X and the bond projecting to the right is bonded to the isoxazole ring in Formula 1; q is 0 or 1.

Embodiment 46. The method of Embodiment 45 wherein R^{Y} is H.
Embodiment 47. The method of Embodiment 45 wherein q is 0.
Embodiment 48. The method of any one of Embodiments 45 through 47 wherein G is selected from G-25 through G-34 and G-43 through G-48.
Embodiment 49. The method of Embodiment 48 wherein G is selected from G-26, G-34, G-43 and G-47.

Embodiments of this invention, including Embodiments 1-49 above as well as any other embodiments described herein, can be combined in any manner, and the descriptions of variables in the embodiments pertain not only to methods of treatment but also to the compounds of Formula 1, starting compounds and intermediate compounds useful for preparing the compounds of Formula 1 and to the compositions comprising the compounds of Formula 1 unless further defined in the Embodiments. Combinations of Embodiments 1-49 are illustrated by:
Embodiment A1. The method described in the Summary of the Invention for treating a subject suffering from or diagnosed with a disease, disorder, or condition mediated by fatty acid amide hydrolase activity, said method comprising administering to the subject in need of such treatment an effective amount of a compound selected from compounds of Formula 1 wherein
   A is O or NH;
   R¹ is selected from U-1 through U-51 as shown in Exhibit 1 wherein each R^{V} is independently selected from H and R^{5a} when R^{V} is attached to a carbon atom ring member, and R^{V} is selected from H and R^{5b} when R^{V} is attached to a nitrogen atom ring member, and the bond projecting to the left is bonded to A of Formula 1;
   k is 0, 1, 2 or 3;
   R⁴ is benzyl, phenyl or naphthalenyl, each optionally substituted with up to 3 substituents independently selected from R^{8a}; or pyridinyl, thienyl, pyrazolyl, triazolyl or imidazolyl, each optionally substituted with up to 3 substituents independently selected from R^{8a} on carbon atom ring members and R^{8b} on a nitrogen atom ring member;
   G is selected from G-1 through G-48 as shown in Exhibit 2 wherein R^{Y} is selected from H and R^{7a} when R^{Y} is attached to a carbon atom ring member, and R^{Y} is selected from H and R^{7b} when R^{Y} is attached to a nitrogen atom ring member, and the bond projecting to the left is bonded to X and the bond projecting to the right is bonded to the isoxazole ring in Formula 1; and
   q is 0 or 1.
Embodiment A2. A method of Embodiment A1 wherein
   A is O;
   W is O;
   X is CR^{2a};
   R¹ is selected from U-21 and U-37 through U-51;
   each R² is independently C₁-C₂ alkyl or C₁-C₂ haloalkyl;
   R^{2a} is H;
   each R³ is independently cyano or C₁-C₃ alkyl;
   R⁴ is benzyl or phenyl, each optionally substituted with up to 3 substituents independently selected from R^{8a}; or pyridinyl or thienyl, each optionally substituted with up to 3 substituents independently selected from R^{8a} on carbon atom ring members;
   each R^{5a} is independently halogen, hydroxy, cyano, nitro, C₁-C₄ alkyl, C₁-C₆ haloalkyl, C₁-C₄ alkoxy, C₁-C₄ haloalkoxy, C₁-C₄ alkylthio, C₁-C₄ haloalkylthio, C₁-C₄ alkylsulfonyl, C₁-C₄ alkylsulfonyl, C₁-C₄ haloalkylsulfinyl, C₁-C₄ haloalkylsulfonyl, C₂-C₈ dialkylamino, C₂-C₄ alkylcarbonyl, C₂-C₆ alkoxycarbonyl or C₂-C₆ alkylcarbonyloxy;
   G is selected from G-25 through G-34 and G-43 through G-48;
   each R^{8a} is independently halogen, hydroxy, amino, cyano, nitro, C₁-C₃ alkyl, C₁-C₃ haloalkyl, C₁-C₃ alkoxy, C₁-C₃ haloalkoxy, C₁-C₃ alkylthio or C₁-C₃ haloalkylthio;
   n is 0 or 1; and
   q is 0.
Embodiment A3. A method of Embodiment A2 wherein
   R¹ is selected from U-21, U-37, U-38, U-39, U-42, U-44, U-50 and U-51;
   R⁴ is a phenyl optionally substituted with up to 3 substituents independently selected from R^{8a};
   each R^{5a} is independently halogen, cyano, nitro, C₁-C₂ alkyl, C₁-C₂ haloalkyl, C₁-C₂ alkoxy or C₁-C₂ haloalkoxy;
   n is 0; and
   m is 0 or 1.
Embodiment A4. A method of Embodiment A3 wherein
   R¹ is selected from U-21, U-50 and U-51;
   R³ is cyano or C₁-C₂ alkyl;
   each R^{5a} is independently halogen, nitro, C₁-C₂ alkyl, C₁-C₂ haloalkyl or C₁-C₂ alkoxy; and
   G is selected from G-26, G-34, G-43 and G-47.
Embodiment A5. A method of Embodiment A4 wherein
   R¹ is U-50;
   R⁴ is a phenyl;
   each R^{5a} is independently bromo, chloro, methyl, trifluoromethyl or methoxy;
   G is G-26; and
   m is 0.

Specific embodiments include a method described in the Summary of the Invention for treating a subject suffering from or diagnosed with a disease, disorder, or condition mediated by fatty acid amide hydrolase activity, said method comprising administering to the subject in need of such treatment an effective amount of a compound of Formula 1 selected from the group consisting of:
phenyl-4-[4-(4,5-dihydro-5-phenyl-3-isoxazolyl)-2-thiazolyl]-1-piperidinecarboxylate; and
2-chlorophenyl-4-[4-(4,5-dihydro-5-phenyl-3-isoxazolyl)-2-thiazolyl]-1-piperidine-carboxylate.

Embodiments of the present invention also include Embodiments B1 through B35 described below.
Embodiment B1. A compound of Formula 1 wherein
   A is O or S;
   W is O or S;
   X is CR^{2a} or N;
   R¹ is phenyl, naphthalenyl or 1,2-benzisoxazol-3-yl, each optionally substituted with up to 3 substituents independently selected from R^{5a}; or a 5- to 6-membered heteroaromatic ring, the ring containing ring members selected from carbon atoms and 1 to 4 heteroatoms independently selected from up to 2 O, up to 2 S and up to 4 N atoms, the ring optionally substituted with up to 3 substituents independently selected from R^{5a} on carbon atom ring members and R^{5b} on nitrogen atom ring members;
   each R² is independently halogen, cyano, hydroxy, C₁-C₂ alkyl, C₁-C₂ haloalkyl or C₁-C₂ alkoxy;
   R^{2a} is H, halogen, cyano, hydroxy, C₁-C₂ alkyl, C₁-C₂ haloalkyl or C₁-C₂ alkoxy;
   each R³ is independently halogen, cyano, C₁-C₃ alkyl or C₁-C₃ haloalkyl;
   R⁴ is C₁-C₈ alkyl, C₁-C₈ haloalkyl, C₃-C₈ cycloalkyl, C₃-C₈ halocycloalkyl, C₄-C₁₀ alkylcycloalkyl, C₄-C₁₀ cycloalkylalkyl, C₂-C₈ alkoxyalkyl, C₂-C₈ haloalkoxyalkyl, C₄-C₁₀ cycloalkoxyalkyl, C₃-C₈ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₂-C₆ haloalkylaminoalkyl, C₃-C₈ dialkylaminoalkyl, C₄-C₁₀ cycloalkylaminoalkyl, C₁-C₆ hydroxyalkyl, C₂-C₆ alkylcarbonyl, C₂-C₆ haloalkylcarbonyl, C₂-C₆ alkoxycarbonyl, C₂-C₆ alkylaminocarbonyl or C₃-C₈ dialkylaminocarbonyl; or benzyl, phenyl, naphthalenyl, 1,3-dihydro-1,3-dioxo-2H-isoindol-2-yl, 2-oxo-3(2H)-benzooxazol-3-yl or 2-oxo-3(2H)-benzothiazol-3-yl or each optionally substituted with up to 3 substituents independently selected from R^{8a}; or a 5- to 6-membered heteroaromatic ring, the ring optionally substituted with up to 3 substituents independently selected from R^{8a} on carbon atom ring members and R^{8b} on nitrogen atom ring members;
   each R^{5a} is independently halogen, hydroxy, amino, cyano, nitro, C₁-C₄ alkyl, C₁-C₆ haloalkyl, C₃-C₆ cycloalkyl, C₃-C₆ halocycloalkyl, C₂-C₄ alkoxyalkyl, C₁-C₄ hydroxyalkyl, C₁-C₄ alkoxy, C₁-C₄ haloalkoxy, C₁-C₄ alkylthio, C₁-C₄ haloalkylthio, C₁-C₄ alkylsulfinyl, C₁-C₄ alkylsulfonyl, C₁-C₄ haloalkylsulfinyl, C₁-C₄ haloalkylsulfonyl, C₁-C₄ alkylamino, C₂-C₈ dialkylamino, C₂-C₄ alkylcarbonyl, C₂-C₆ alkoxycarbonyl, C₂-C₆ alkylaminocarbonyl, C₃-C₈ dialkylaminocarbonyl, C₂-C₆ alkylcarbonyloxy, C₂-C₆ alkylcarbonylthio or C₃-C₆ trialkylsilyl;
   each R^{5b} is independently C₁-C₄ alkyl, C₃-C₄ alkenyl, C₃-C₄ alkynyl, C₃-C₆ cycloalkyl, C₁-C₄ haloalkyl, C₃-C₄ haloalkenyl, C₃-C₄ haloalkynyl, C₃-C₆ halocycloalkyl or C₂-C₄ alkoxyalkyl;
   G is a 5-membered heteroaromatic ring, the ring containing ring members selected from carbon atoms and 1 to 3 heteroatoms independently selected from up to 2 O, up to 2 S and up to 3 N atoms, the ring optionally substituted with up to 1 substituent selected from R^{7a} on a carbon atom and R^{7b} on a nitrogen atom;
   R^{7a} is halogen, cyano, C₁-C₂ alkyl or C₁-C₂ haloalkyl;
   R^{7b} is C₁-C₂ alkyl or C₁-C₂ haloalkyl;
   each R^{8a} is independently halogen, hydroxy, amino, cyano, nitro, C₁-C₄ alkyl, C₁-C₄ haloalkyl, C₁-C₄ alkoxy, C₁-C₄ haloalkoxy, C₁-C₄ alkylthio, C₁-C₄ haloalkylthio, C₁-C₄ alkylsulfinyl, C₁-C₄ alkylsulfonyl, C₁-C₄ haloalkylsulfinyl, C₁-C₄ haloalkylsulfonyl, C₁-C₄ alkylamino, C₂-C₆ dialkylamino, C₂-C₄ alkylcarbonyl, C₂-C₆ alkoxycarbonyl, C₂-C₆ alkylaminocarbonyl or C₃-C₈ dialkylaminocarbonyl; or
   a pair of R^{8a} and R³ are taken together with the atoms to which they are attached to form a 5- to 7-membered ring, the ring containing ring members selected from carbon atoms and up to 2 heteroatoms independently selected from up to 1 O, up to 1 S and up to 1 N, wherein up to 2 carbon atom ring members are independently selected from C(═O) and C(═S), and the sulfur atom ring members are independently selected from)S(═O)ᵤ(═NR¹⁰)_{z}, the ring optionally substituted with up to 2 substituents independently selected from R^{9a} on carbon atom ring members and from R^{9b} on a nitrogen atom ring member;
   each R^{8b} is independently C₁-C₄ alkyl or C₁-C₄ haloalkyl; or
   a pair of R^{8b} and R³ are taken together with the atoms to which they are attached to form a 5- to 7-membered ring, the ring containing ring members selected from carbon atoms and up to 2 heteroatoms independently selected from up to 1 O, up to 1 S and up to 1 N, wherein up to 2 carbon atom ring members are independently selected from C(═O) and C(═S), and the sulfur atom ring members are independently selected from)S(═O)ᵤ(═NR¹⁰)_{z}, the ring optionally substituted with up to 2 substituents independently selected from R^{9a} on carbon atom ring members and from R^{9b} on a nitrogen atom ring member;
   each R^{9a} is independently halogen, C₁-C₄ alkyl, C₁-C₄ haloalkyl, C₁-C₄ alkoxy, C₁-C₄ haloalkoxy, C₁-C₄ alkylthio or C₁-C₄ haloalkylthio;
   R^{9b} is C₁-C₄ alkyl or C₁-C₄ haloalkyl;
   R¹⁰ is independently H, C₁-C₄ alkyl, C₂-C₄ alkenyl, C₂-C₄ alkynyl, C₁-C₄ haloalkyl, C₂-C₄ haloalkenyl, C₂-C₄ haloalkynyl, C₂-C₄ alkoxyalkyl, C₂-C₄ alkylcarbonyl, C₂-C₄ haloalkylcarbonyl, C₁-C₄ alkylsulfonyl or C₁-C₄ haloalkylsulfonyl;
   m is 0, 1 or 2;
   n is 0, 1 or 2; and
   u and z in the instance of)S(═O)ᵤ(═NR¹⁰)_{z} are independently 0, 1 or 2, provided that the sum of u and z in the instance of)S(═O)ᵤ(═NR¹⁰)_{z} is 0, 1 or 2;
   provided that when X is N, then G is attached to X through a carbon atom ring member.
Embodiment B2. A compound of Embodiment B1 wherein A is O.
Embodiment B3. A compound of Embodiment B1 or B2 wherein W is O. Embodiment B4. A compound of any one of Embodiments B1 through B3 wherein X is CR^{2a} or N.
Embodiment B5. A compound of Embodiment B4 wherein X is N.
Embodiment B6. A compound of Embodiment B4 wherein X is CR^{2a}.

Embodiment B7. A compound of Embodiment B6 wherein R^{2a} is H.
Embodiment B8. A compound of any one of Embodiments B1 through B7 wherein R¹ is selected from U-1 through U-51 as shown in Exhibit 1

### Exhibit 1

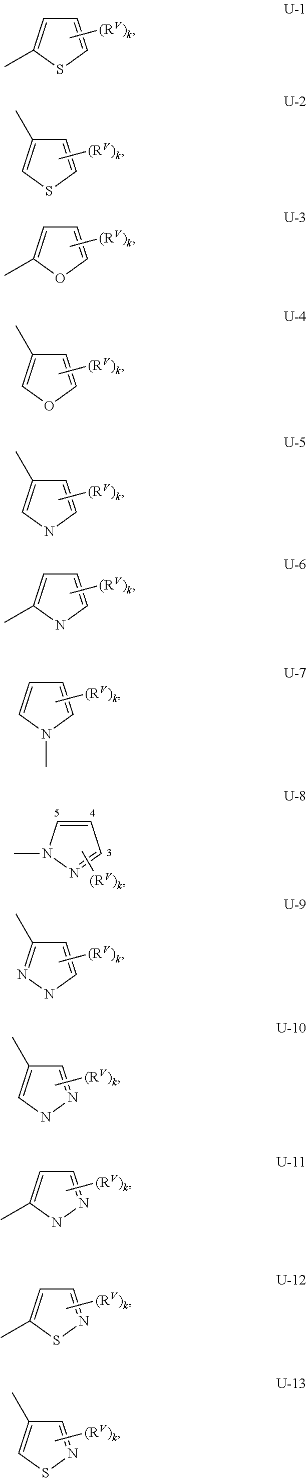 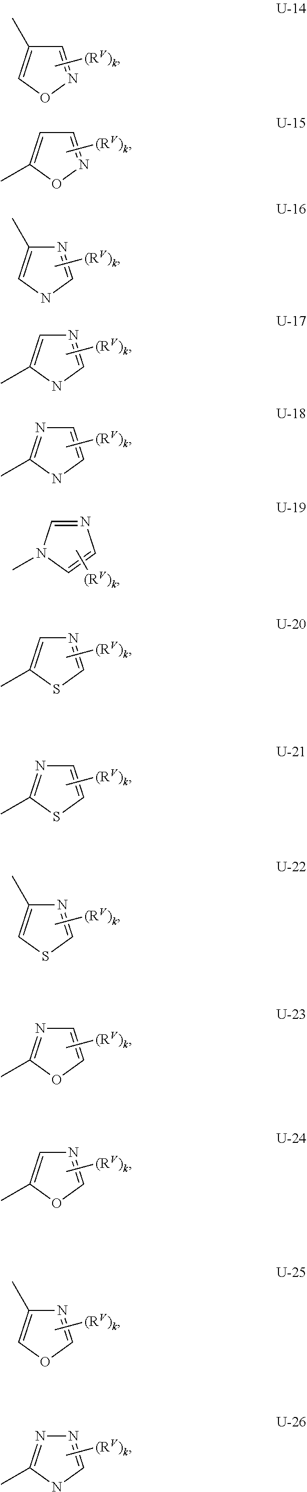 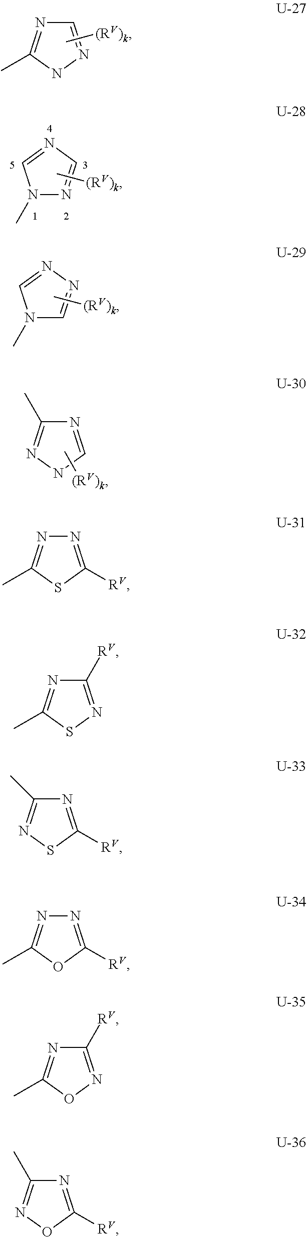 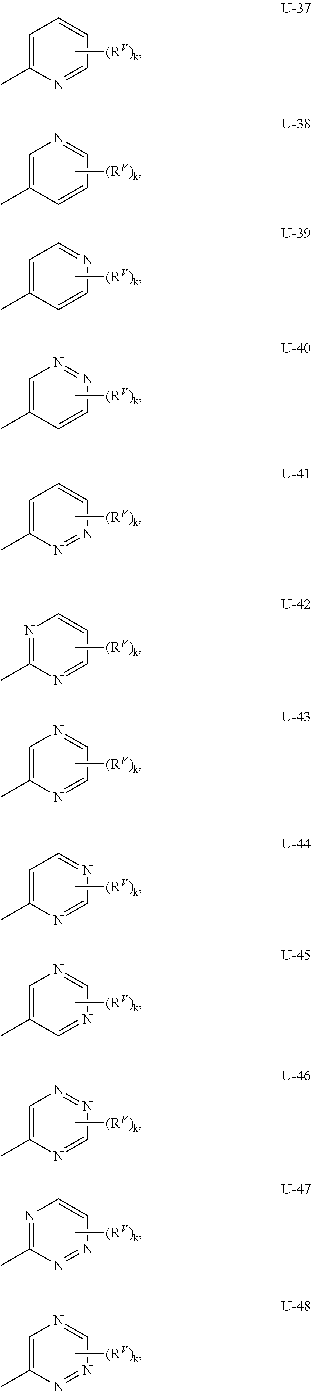 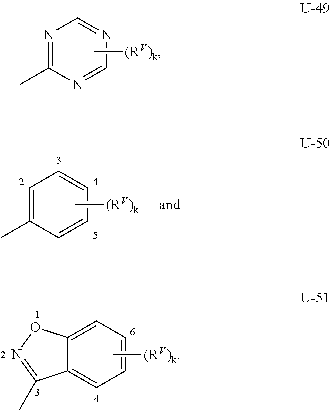 [0232] wherein each R^{V} is independently selected from H and R^{5a} when R^{V} is attached to a carbon atom ring member, and R^{V} is selected from H and R^{5b} when R^{V} is attached to a nitrogen atom ring member (e.g., U-5, U-6, U-9, U-10, U-11, U-16, U-17, U-18, U-26, U-27 or U-30), and the bond projecting to the left is bonded to A of Formula 1; k is 0, 1, 2 or 3.
Embodiment B9. A compound of any one of Embodiments B1 through B8 wherein each R^{5a} is independently halogen, hydroxy, cyano, nitro, C₁-C₄ alkyl, C₁-C₆ haloalkyl, C₁-C₄ alkoxy, C₁-C₄ haloalkoxy, C₁-C₄ alkylthio, C₁-C₄ haloalkylthio, C₁-C₄ alkylsulfinyl, C₁-C₄ alkylsulfonyl, C₁-C₄ haloalkylsulfinyl, C₁-C₄ haloalkylsulfonyl, C₂-C₈ dialkylamino, C₂-C₄ alkylcarbonyl, C₂-C₆ alkoxycarbonyl or C₂-C₆ alkylcarbonyloxy.
Embodiment B10. A compound of Embodiment B9 wherein each R^{5a} is independently halogen, cyano, nitro, C₁-C₂ alkyl, C₁-C₂ haloalkyl, C₁-C₂ alkoxy or C₁-C₂ haloalkoxy.
Embodiment B11. A compound of Embodiment B10 wherein each R^{5a} is independently bromo, chloro, methyl, trifluoromethyl or methoxy.
Embodiment B12. A compound of any one of Embodiment B11 wherein each R^{5a} is independently chloro, methyl, trifluoromethyl or methoxy
Embodiment B13. A compound any one of Embodiments B1 through B12 wherein each R^{5b} is independently C₁-C₄ alkyl, C₁-C₄ haloalkyl or C₂-C₄ alkoxyalkyl.
Embodiment B14. A compound of Embodiment B13 wherein each R^{5b} is independently C₁-C₄ alkyl.
Embodiment B15. A compound of Embodiment B14 wherein each R^{5b} is methyl.
Embodiment B16. A compound of Embodiments B8 wherein each R^{V} is H.
Embodiment B17. A compound of Embodiments B8 wherein each k is 0.
Embodiment B18. A compound of any one of Embodiments B8 through B17 wherein R¹ is selected from U-21 and U-37 through U-51.
Embodiment B19. A compound of Embodiment B18 wherein R¹ is selected from U-21, U-37, U-38, U-39, U-42, U-44, U-50 and U-51.
Embodiment B20. A compound of Embodiment B19 wherein R¹ is selected from U-21, U-50 and U-51.
Embodiment B21. A compound of any one of Embodiments B1 through B20 wherein each R² is independently C₁-C₂ alkyl or C₁-C₂ haloalkyl.
Embodiment B22. A compound of any one of Embodiments B1 through B21 wherein n is 0 or 1.
Embodiment B23. A compound of Embodiment B22 wherein n is 0.
Embodiment B24. A compound of any one of Embodiments B1 through B23 wherein each R³ when taken alone (i.e. not taken together with R^{8a} or R^{8b}) is independently cyano or C₁-C₃ alkyl.
Embodiment B25. A compound of Embodiment B24 wherein each R³ when taken alone is independently cyano or C₁-C₂ alkyl.
Embodiment B26. A compound of any one of Embodiments B1 through B25 wherein each R³ is taken alone (i.e. not taken together with R^{8a} or R^{8b}).
Embodiment B27. A compound of any one of Embodiments B1 through B26 wherein m when is 0 or 1.
Embodiment B28. A compound of any one of Embodiments B1 through B27 wherein R⁴ is benzyl, phenyl or naphthalenyl, each optionally substituted with up to 3 substituents independently selected from R^{8a}; or pyridinyl, thienyl, pyrazolyl, triazolyl or imidazolyl, each optionally substituted with up to 3 substituents independently selected from R^{8a} on carbon atom ring members and R^{8b} on a nitrogen atom ring member.
Embodiment B29. A compound of Embodiment B28 wherein R⁴ is benzyl or phenyl, each optionally substituted with up to 3 substituents independently selected from R^{8a}; or pyridinyl or thienyl, each optionally substituted with up to 3 substituents independently selected from R^{8a} on carbon atom ring members.
Embodiment B30. A compound of Embodiment B29 wherein R⁴ is a phenyl optionally substituted with up to 3 substituents independently selected from R^{8a}.
Embodiment B31. A compound of any one of Embodiments B1 through B30 wherein each R^{8a} when taken alone (i.e. not taken together with R³) is independently halogen, hydroxy, amino, cyano, nitro, C₁-C₃ alkyl, C₁-C₃ haloalkyl, C₁-C₃ alkoxy, C₁-C₃ haloalkoxy, C₁-C₃ alkylthio or C₁-C₃ haloalkylthio.
Embodiment B32. A compound of Embodiment B31 wherein each R^{8a} when taken alone is independently halogen, methyl, halomethyl or methoxy.
Embodiment B33. A compound of any one of Embodiments B1 through B32 wherein each R^{8a} is taken alone (i.e. not taken together with R³).
Embodiment B34. A compound of any one of Embodiments B1 through B33 wherein G is selected from G-1 through G-48 as shown in Exhibit 2

### Exhibit 2

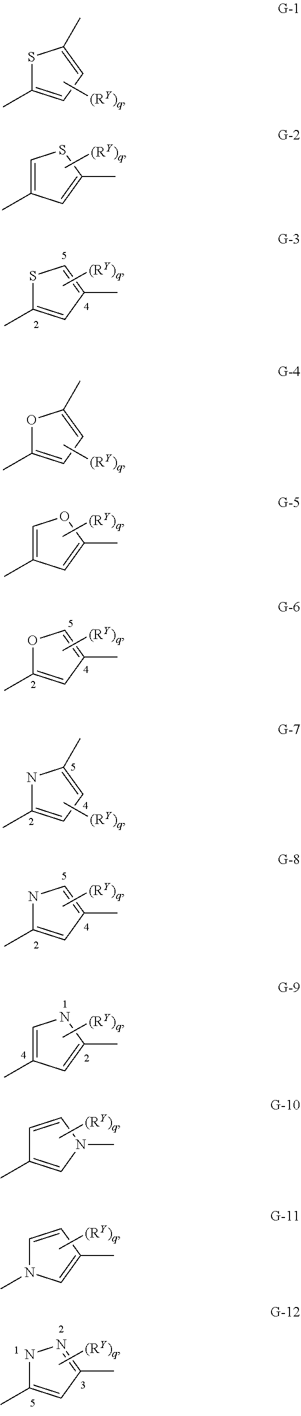 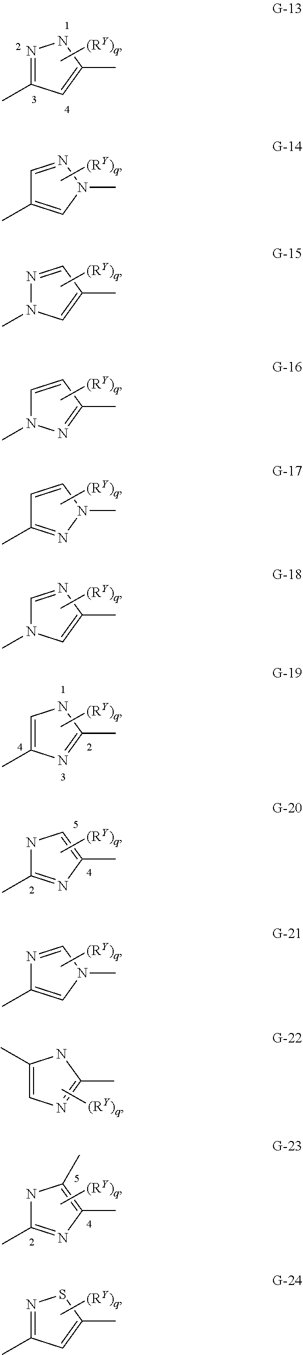 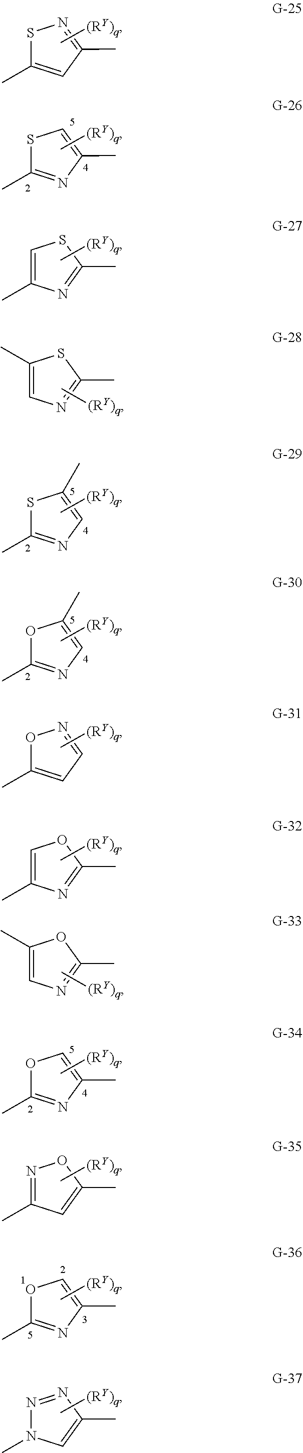 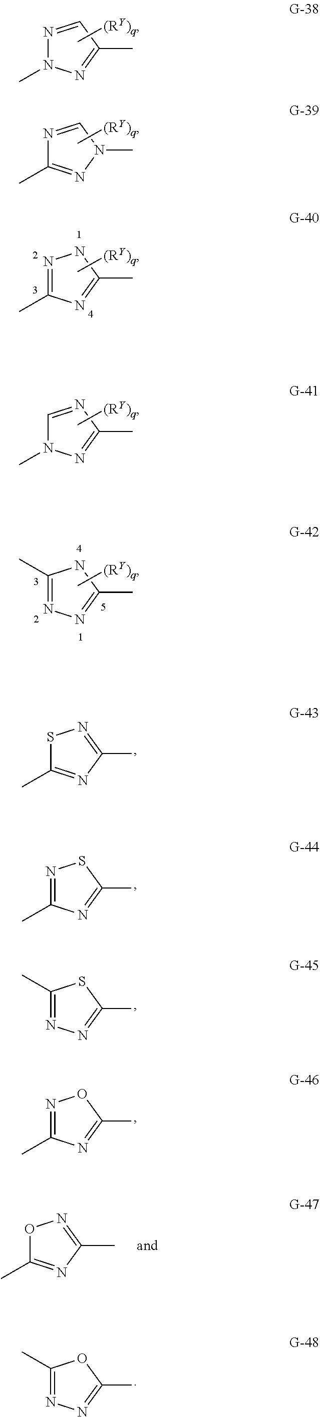 [0260] wherein R^{Y} is selected from H and R^{7a}, when R^{Y} is attached to a carbon atom ring member, and R^{Y} is selected from H and R^{7b} when R^{Y} is attached to a nitrogen atom ring member, and the bond projecting to the left is bonded to X and the bond projecting to the right is bonded to the isoxazole ring in Formula 1; q is 0 or 1;
Embodiment B35. A compound of Embodiment B34 wherein G is selected from G-25 through G-34 and G-43 through G-48.
Embodiment B36. A compound of Embodiment B35 wherein G is selected from G-26, G-34, G-43 and G-47.
Embodiment B37. A compound of Embodiment B36 wherein R^{Y} is H.
Embodiment B38. A compound of any one of Embodiment B31 wherein q is 0.
   Combinations of Embodiments B1-B38 are illustrated by:
Embodiment C1. A compound of Embodiment B1 wherein
   R¹ is selected from U-1 through U-51 as shown in Exhibit 1 wherein each R^{V} is independently selected from H and R^{5a} when R^{V} is attached to a carbon atom ring member, and R^{V} is selected from H and R^{5b} when R^{V} is attached to a nitrogen atom ring member, and the bond projecting to the left is bonded to A of Formula 1;
   k is 0, 1, 2 or 3;
   R⁴ is benzyl, phenyl or naphthalenyl, each optionally substituted with up to 3 substituents independently selected from R^{8a}; or pyridinyl, thienyl, pyrazolyl, triazolyl or imidazolyl, each optionally substituted with up to 3 substituents independently selected from R^{8a} on carbon atom ring members and R^{8b} on a nitrogen atom ring member;
G is selected from G-1 through G-48 as shown in Exhibit 2 wherein R^{Y} is selected from H and R^{7a} when R^{Y} is attached to a carbon atom ring member, and R^{Y} is selected from H and R^{7b} when R^{Y} is attached to a nitrogen atom ring member, and the bond projecting to the left is bonded to X and the bond projecting to the right is bonded to the isoxazole ring in Formula 1; and
q is 0 or 1.
Embodiment C2. A compound of Embodiment C1 wherein
   A is O;
   W is O;
   X is CR^{2a};
   R¹ is selected from U-21 and U-37 through U-51;
   each R² is independently C₁-C₂ alkyl or C₁-C₂ haloalkyl;
   R^{2a} is H;
   each R³ is independently cyano or C₁-C₃ alkyl;
   R⁴ is benzyl or phenyl, each optionally substituted with up to 3 substituents independently selected from R^{8a}; or pyridinyl or thienyl, each optionally substituted with up to 3 substituents independently selected from R^{8a} on carbon atom ring members;
   each R^{5a} is independently halogen, hydroxy, cyano, nitro, C₁-C₄ alkyl, C₁-C₆ haloalkyl, C₁-C₄ alkoxy, C₁-C₄ haloalkoxy, C₁-C₄ alkylthio, C₁-C₄ haloalkylthio, C₁-C₄ alkylsulfonyl, C₁-C₄ alkylsulfonyl, C₁-C₄ haloalkylsulfinyl, C₁-C₄ haloalkylsulfonyl, C₂-C₈ dialkylamino, C₂-C₄ alkylcarbonyl, C₂-C₆ alkoxycarbonyl or C₂-C₆ alkylcarbonyloxy;
   G is selected from G-25 through G-34 and G-43 through G-48;
   each R^{8a} is independently halogen, hydroxy, amino, cyano, nitro, C₁-C₃ alkyl, C₁-C₃ haloalkyl, C₁-C₃ alkoxy, C₁-C₃ haloalkoxy, C₁-C₃ alkylthio or C₁-C₃ haloalkylthio;
   n is 0 or 1; and
   q is 0.
Embodiment C3. A compound of Embodiment C2 wherein
   R¹ is selected from U-21, U-37, U-38, U-39, U-42, U-44, U-50 and U-51;
   each R^{5a} is independently halogen, cyano, nitro, C₁-C₂ alkyl, C₁-C₂ haloalkyl, C₁-C₂ alkoxy or C₁-C₂ haloalkoxy;
   R⁴ is a phenyl ring optionally substituted with up to 3 substituents independently selected from R^{8a};
   n is 0; and
   m is 0 or 1.
Embodiment C4. A compound of Embodiment C3 wherein
   R¹ is selected from U-21, U-50 and U-51;
   R³ is cyano or C₁-C₂ alkyl;
   each R^{5a} is independently halogen, nitro, C₁-C₂ alkyl, C₁-C₂ haloalkyl or C₁-C₂ alkoxy; and
   G is selected from G-26, G-34, G-43 and G-47.
Embodiment C5. A compound of Embodiment C4 wherein
   R¹ is U-50;
   R⁴ is a phenyl;
   each R^{5a} is independently bromo, chloro, methyl, trifluoromethyl or methoxy;
   G is G-26; and
   m is 0.

Specific embodiments include compounds of Formula 1 selected from the group consisting of:
phenyl-4-[4-(4,5-dihydro-5-phenyl-3-isoxazolyl)-2-thiazolyl]-1-piperidinecarboxylate; and
2-chlorophenyl-4-[4-(4,5-dihydro-5-phenyl-3-isoxazolyl)-2-thiazolyl]-1-piperidine-carboxylate.

One or more of the following methods and variations as described in Schemes 1-12 can be used to prepare the compounds of Formula 1. The definitions of (R¹, R², R³, R⁴, A, W, X, G, n and m) in the compounds of Formulae 1-26 below are as defined above in the Summary of the Invention unless otherwise noted.

As shown in Scheme 1, compounds of Formula 1 wherein A is O, S or NR⁶ and R⁶ is other than H can be prepared by coupling a chloroformate, thiochloroformate, carbamoyl chloride or thiocarbamoyl chloride of Formula 2 with an amine of Formula 3 in the presence of an acid scavenger. Typical acid scavengers include amine bases such as triethylamine, N,N-diisopropylethylamine and pyridine. Other scavengers include hydroxides such as sodium and potassium hydroxide and carbonates such as sodium carbonate and potassium carbonate. In certain instances it is useful to use polymer-supported acid scavengers such as polymer-bound N,N-diisopropylethylamine and polymer-bound 4-(dimethylamino)pyridine. Acid salts of the Formula 3 amines can also be used in this reaction, provided that at least 2 equivalents of the acid scavenger is present. Typical acids used to form salts with amines include hydrochloric acid, oxalic acid and trifluoroacetic acid. In a subsequent step, carbamates and ureas of Formula 1 wherein W is O can be converted to thiocarbamates and thioureas of Formula 1 wherein W is S using a variety of standard thiating reagents such as phosphorus pentasulfide or 2,4-bis(4-methoxyphenyl)-1,3-dithia-2,4-diphosphetane-2,4-disulfide (Lawesson's reagent). The chloroformates, thiochloroformates, carbamoyl chlorides or thiocarbamoyl chlorides of Formula 2 are either known or can be prepared by methods known to one skilled in the art.

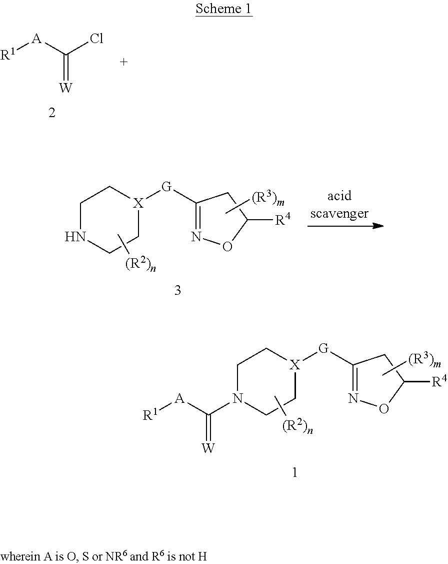

Compounds of Formula 1 can also be prepared by the reaction of an amine, thiol or hydroxyl compound of Formula 4 with a carbamoyl or thiocarbamoyl chloride or imidazole of Formula 5 as shown in Scheme 2. When Y is chlorine, the reaction is typically carried out in the presence of an acid scavenger. Typical acid scavengers include amine bases such as triethylamine, N,N-diisopropylethylamine and pyridine. Other scavengers include hydroxides such as sodium and potassium hydroxide and carbonates such as sodium carbonate and potassium carbonate. The carbamoyl or thiocarbamoyl chlorides of Formula 5 (wherein Y is CO can be prepared from amines of Formula 3 by treatment with phosgene or thiophosgene, respectively, or their equivalents, while carbamoyl or thiocarbamoyl imidazoles of Formula 5 (wherein Y is imidazol-1-yl) can be prepared from amines of Formula 3 by treatment with 1,1′-carbonyldiimidazole or 1,1′-thiocarbonyldiimidazole, respectively, according to general methods known to one skilled in the art. Thiocarbamates can also be formed by palladium-catalyzed reactions of disulfides, amines and carbon monoxide as described by Y. Nishiyama, et al., J. Org. Chem., 2005, 70, 2551-2554. The amine, thiol or hydroxyl compounds of Formula 4 are either known or can be prepared by one skilled in the art.

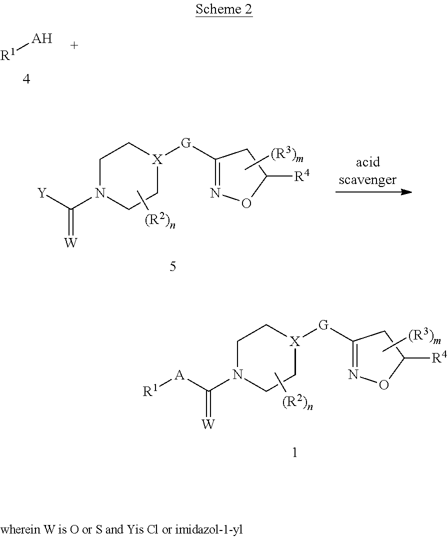

Compounds of Formula 1 wherein A is NH, can be prepared by reaction of an amine of Formula 3 with an isocyanate or isothiocyanate, respectively, of Formula 6 as depicted in Scheme 3. This reaction is typically carried out at ambient temperature in an aprotic solvent such as dichloromethane or acetonitrile.

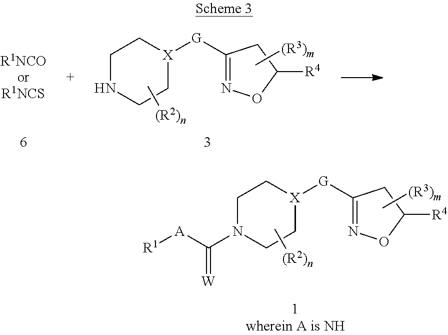

Certain compounds of Formula 1 wherein X is CR² and G is linked to the ring containing X via a nitrogen atom, can be prepared by displacement of an appropriate leaving group Y¹ on the ring containing the X of Formula 7 with a nitrogen-containing heterocycle of Formula 8 in the presence of a base as depicted in Scheme 4. Suitable bases include sodium hydride or potassium carbonate, and the reaction is carried out in a solvent such as N,N-dimethylformamide or acetonitrile at 0 to 80° C. Suitable leaving groups in the compounds of Formula 7 include bromide, iodide, mesylate (OS(O)₂CH₃), triflate (OS(O)₂CF₃) and the like, and compounds of Formula 7 can be prepared from the corresponding compounds wherein Y¹ is OH, using general methods known in the art.

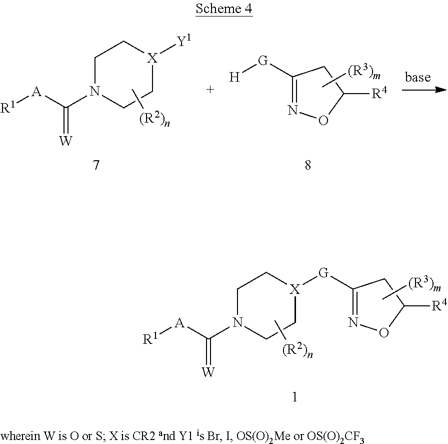

Compounds of Formula 1 wherein X is N can be prepared by reaction of a compound of Formula 9 with a heterocyclic halide or triflate (OS(O)₂CF₃) of Formula 10 as shown in Scheme 5. The reaction is carried out in the presence of a base such as potassium carbonate in a solvent such as dimethylsulfoxide, N,N-dimethylformamide or acetonitrile at 0 to 80° C. Compounds of Formula 10 wherein Y¹ is triflate can be prepared from corresponding compounds wherein Y¹ is OH by methods known to one skilled in the art.

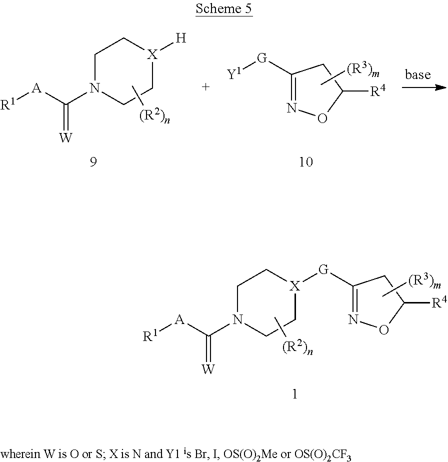

Compounds of Formula 1 can also be prepared by reaction of a suitably functionalized compound of Formula 11 with a suitably functionalized compound of Formula 12 as shown in Scheme 6. The functional groups Y² and Y³ are selected from, but not limited to, moieties such as aldehydes, ketones, esters, acids, amides, thioamides, nitriles, amines, alcohols, thiols, hydrazines, oximes, amidines, amideoximes, olefins, acetylenes, halides, alkyl halides, methanesulfonates, trifluoromethanesulfonates, boronic acids, boronates, and the like, which under the appropriate reaction conditions, will allow the construction of the various heterocyclic rings G. As an example, reaction of a compound of Formula 11 where Y² is a thioamide group with a compound of Formula 12 where Y³ is a bromoacetyl group will give a compound of Formula 1 where G is a thiazole ring. The synthetic literature describes many general methods for forming 5-membered heteroaromatic rings (e.g., G-1 through G-48 of Exhibit 2); see, for example, Comprehensive Heterocyclic Chemistry, Vol. 4-6, A. R. Katritzky and C. W. Rees editors, Pergamon Press, New York, 1984; Comprehensive Heterocyclic Chemistry II, Vol. 2-4, A. R. Katritzky, C. W. Rees, and E. F. Scriven editors, Pergamon Press, New York, 1996; and the series, The Chemistry of Heterocyclic Compounds, E. C. Taylor, editor, Wiley, New York. The use of intermediates of Formula 11 where X is a carbon atom and Y² is Br, I, methanesulfonate or trifluoromethanesulfonate to prepare organozinc reagents for use in cross-coupling reactions with aromatic rings has been described; see, for example, S. Bellotte, Synlett 1998, 379-380, and M. Nakamura et al., Synlett 2005, 1794-1798. One skilled in the art knows how to select the appropriate functional groups to construct the desired heterocyclic ring G.

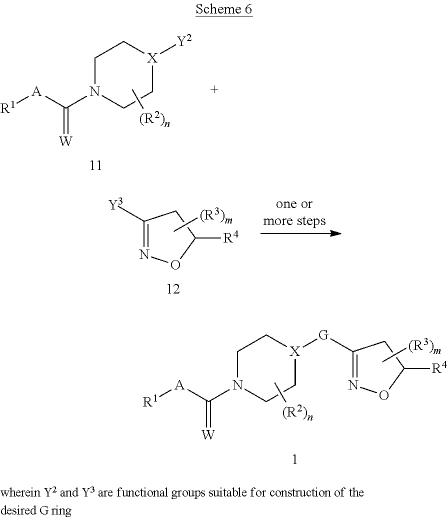

Compounds of Formula 1 where G is linked to the isoxazoline ring via a nitrogen atom can be prepared by displacement of halogen leaving group Y⁴ on an isoxazoline of Formula 14 with a compound of Formula 13 in the presence of a base as depicted in Scheme 7. Suitable bases include sodium hydride or potassium carbonate, and the reaction is carried out in a solvent such as N,N-dimethylformamide or acetonitrile at temperatures between about 0 to 80° C. Compounds of Formula 14 are known or can be prepared by reacting a dihaloformaldoxime with an appropriate vinyl compound as known in the art.

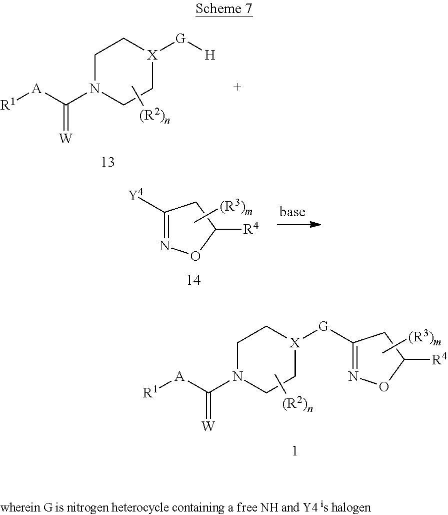

Compounds of Formula 1 can also be prepared by reaction of a chloro oxime of Formula 15 with a olefin of Formula 16 in the presence of a base as shown in Scheme 8. The reaction proceeds via an intermediate nitrile oxide. General procedures for cycloaddition of nitrile oxides with olefins are well documented in the chemical literature. For relevant references see Lee, Synthesis 1982, 6, 508-509 and Kanemasa et al., Tetrahedron 2000, 56, 1057-1064 as well as references cited within. The chloro oximes of Formula 15 can be prepare by treating the corresponding aldehyde with hydroxylamine followed by chlorination with a suitable chlorinating agent such as N-chlorosuccinamide, as known to one skilled in the art. The compounds of Formula 16 are known or can be prepared by general methods known in the art.

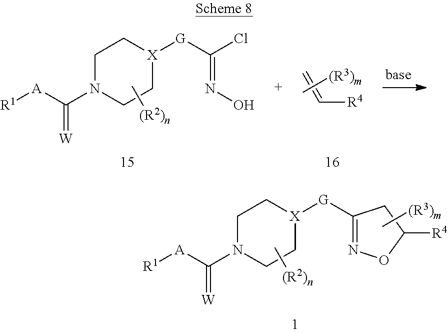

Amines of Formula 3 can be prepared from compounds of Formula 17 wherein Y⁵ is an amine protecting group via a deprotection reaction as shown in Scheme 9. A wide array of amine protecting groups are suitable for the method of Scheme 9 (see, for example, T. W. Greene and P. G. M. Wuts, Protective Groups in Organic Synthesis, 2nd ed.; Wiley: New York, 1991), and the choice of the appropriate protecting groups will be apparent to one skilled in chemical synthesis. After deprotection, the amine of Formula 3 can be isolated as its acid salt or the free amine by general methods known in the art.

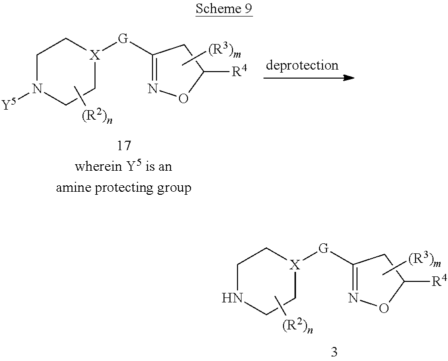

One skilled in the art will recognize that many compounds of Formula 17 can be prepared by methods analogous to those described in Schemes 4 through 8 above where the group R¹A(C═W)— is replaced by Y⁵. Thus, compounds corresponding to Formulae 7, 9, 11, 13 and 15 in which the group R¹A(C═W)— is replaced by Y⁵ are useful intermediates for the preparation of compounds of Formula 1.

Thioamides of Formula 18 are particularly useful intermediates for preparing compounds of Formula 1 and 17. A thioamide of Formula 18 can be prepared by the addition of hydrogen sulfide to the corresponding nitrile of Formula 19 wherein X is a carbon atom and Y⁷ is a nitrile moiety as shown in Scheme 10. The methods of Scheme 10 can be carried out by contacting a compound of Formula 19 with hydrogen sulfide in the presence of an amine such as pyridine, diethylamine or diethanolamine. Alternatively, hydrogen sulfide can be used in the form of its bisulfide salt with an alkali metal or ammonia. This type of reaction is well documented in the literature see; for example, European Patent EP 696581.

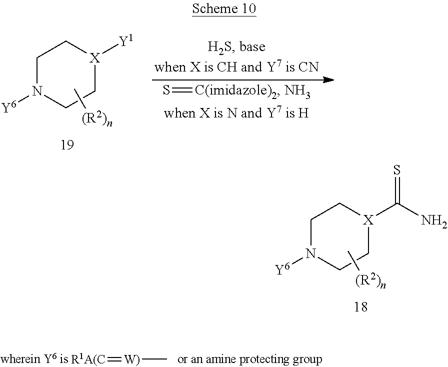

As also shown in Scheme 10, a thioamide of Formula 18 can be prepared by the reaction of a compound of Formula 19 wherein X is a nitrogen atom and Y⁷ is H and thiocarbonyl diimidazole followed by treatment with ammonia as described by J. L. Collins, et al., J. Med. Chem. 1998, 41, 5037-5054.

Halomethyl isoxazoline ketones of Formula 24 are also particularly useful intermediates for preparing certain chiral compounds of Formula 1. Halomethyl isoxazoline ketones of Formula 24 can be prepared by the multi-step reaction sequences shown in Scheme 11.

One skilled in the art will recognize that Scheme 11 can also be practiced without the use of a resolving agent, so that a compound of Formula 21 is converted directly to a racemic analog of Formula 20a, which can then be used to prepare racemic analogs of Formulae 23 and 24 and certain racemic compounds of Formula 1.

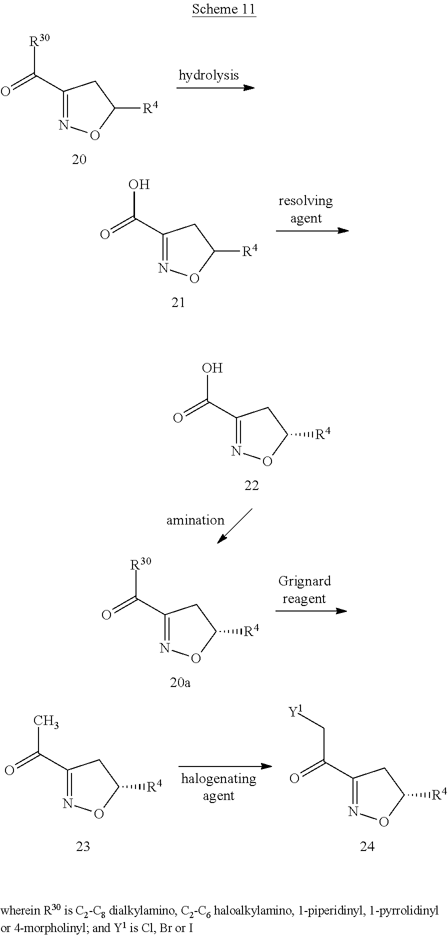

The preparation of racemic carboxylic acids of Formula 21 can be accomplished according to the well-known methods of basic or acidic hydrolysis of the corresponding compounds of Formula 20, preferably using a slight excess of sodium hydroxide in a water-miscible co-solvent such as methanol or tetrahydrofuran at about 25 to 45° C. The product can be isolated by adjusting the pH of the reaction mixture to about 1 to 3 and then filtration or extraction, optionally after removal of the organic solvent by evaporation. The racemic carboxylic acids of Formula 21 can be resolved by classical fractional crystallization of diastereomeric salts of suitable chiral amine bases such as cinchonine, dihydrocinchonine or a mixture thereof. A cinchonine-dihydrocinchonine mixture in about a 85:15 ratio is particularly useful, as it provides, for example, the (R)-configured carboxylic acids of Formula 22, wherein R⁴ is a substituted phenyl group, as the less soluble salt. Furthermore, these chiral amine bases are readily available on a commercial scale. The halomethyl ketones of Formula 24 can be prepared by first reacting the corresponding amides of Formula 20, either as pure enantiomers (i.e. Formula 20a) or in enantiomerically enriched or racemic mixtures, with one molar equivalent of a methylmagnesium halide (Grignard reagent) in a suitable solvent or solvent mixture such as tetrahydrofuran and toluene at about 0 to 20° C., and the crude ketone products of Formula 23 can be isolated by quenching with aqueous acid, extraction, and concentration. Then the crude ketones of Formula 23 are halogenated with a reagent such as sulfuryl chloride to afford the chloromethyl ketones of Formula 24 wherein Y¹ is Cl or molecular bromine to afford the corresponding bromomethyl ketones of Formula 24 wherein Y¹ is Br. The halomethyl ketones of Formula 24 can be purified by crystallization from a solvent such as hexanes or methanol, or can be used without further purification in the condensation reaction with thioamides of Formula 18 to form compounds of Formula 1 where G is a thiazole ring.

The isoxazoline carboxamides of Formula 20 can be prepared by cycloaddition of the corresponding hydroxamoyl chlorides of Formula 25 with olefin derivatives of Formula 26, as shown in Scheme 12.

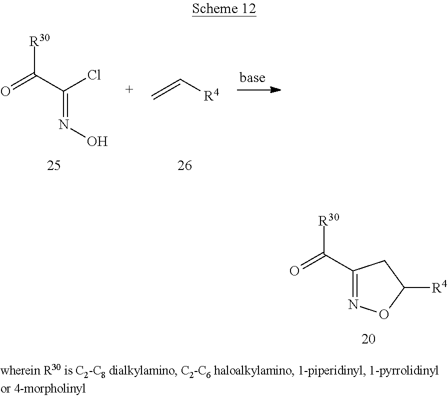

In this method, all three reacting components (the compounds of Formulae 25 and 26, and the base) are contacted so as to minimize hydrolysis or dimerization of the hydroxamoyl chloride of Formula 25. In one typical procedure, the base, which can either be a tertiary amine base such as triethylamine or an inorganic base such as an alkali metal or alkaline-earth carbonate, bicarbonate or phosphate, is mixed with the olefin derivative of Formula 26, and the hydroxamoyl chloride of Formula 25 is added gradually at a temperature at which the cycloaddition proceeds at a relatively rapid rate, typically between 5 and 25° C. Alternatively, the base can be added gradually to the other two components (the compounds of Formulae 25 and 26). This alternative procedure is preferable when the hydroxamoyl chloride of Formula 25 is substantially insoluble in the reaction medium. The solvent in the reaction medium can be water or an inert organic solvent such as toluene, hexane or even the olefin derivative used in excess. The product can be separated from the salt co-product by filtration or washing with water, followed by evaporation of the solvent. The crude product can be purified by crystallization, or the crude product can be used directly in the methods of Scheme 11. Compounds of Formula 20 are useful precursors to the corresponding methyl ketones of Formula 23 and halomethyl ketones of Formula 24, and are also useful for preparing the resolved enantiomers of the compounds of Formulae 23 and 24 by hydrolysis, resolution, methyl ketone synthesis and halogenation, as shown in Scheme 11.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents or the oxidation state within rings.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “q” means quartet, “m” means multiplet, “dd” means doublet of doublets, “dt” means doublet of triplets, “br s” means broad singlet.

### Example 1

### Preparation of phenyl 4-[4-(4,5-dihydro-5-phenyl-3-isoxazolyl)-2-thiazolyl]-1-piperidinecarboxylate (Compound 11)

### Step A: Preparation of 1,1-dimethylethyl 4-[4-(4,5-dihydro-5-phenyl-3-isoxazolyl)-2-thiazolyl]-1-piperidinecarboxylate

To a mixture of 1,1-dimethylethyl 4-(4-formyl-2-thiazolyl)-1-piperidinecarboxylate (1.0 g, 3.4 mmol) in ethanol (5 mL) was added an aqueous solution of hydroxylamine (50 wt. %, 0.25 mL, 4.0 mmol). The reaction mixture was heated at 60° C. for 1 h, during which time the reaction mixture became homogeneous. The resulting reaction solution was cooled to room temperature and diluted with tetrahydrofuran (10 mL). Styrene (0.57 mL, 5 mmol) was added to the reaction mixture, followed by a portionwise addition of Clorox® (aqueous sodium hypochlorite solution) (10.5 mL) over 3 h. The reaction mixture was stirred overnight at room temperature and then filtered. The solid collected by filtration was washed with water and diethyl ether and then air dried to give the title compound as a white powder (610 mg). The filtrate was diluted with saturated aqueous sodium bicarbonate solution and extracted with diethyl ether. The extract was dried (MgSO₄) and concentrated under reduced pressure to give more of the title compound as a yellow oil (850 mg). The oil was diluted with diethyl ether (4 mL) and upon standing provided the title compound as a white solid (233 mg).

¹H NMR (CDCl₃) δ 1.47 (s, 9H), 1.7 (m, 2H), 2.1 (m, 2H), 2.85 (m, 2H), 3.2 (m, 1H), 3.45 (m, 1H), 3.84 (m, 1H) 4.2 (br s, 2H), 5.75 (m, 1H), 7.25-7.40 (m, 5H), 7.61 (s, 1H).

### Step B: Preparation of 4-[4-(4,5-dihydro-5-phenyl-3-isoxazolyl)-2-thiazolyl]piperidine

To a solution of 1,1-dimethylethyl 4-[4-(4,5-dihydro-5-phenyl-3-isoxazolyl)-2-thiazolyl]-1-piperidinecarboxylate (i.e. the product of Step A) (0.815 g, 1.97 mmol) in dichloromethane (50 mL) was added a solution of hydrogen chloride in diethyl ether (2 M, 10 mL, 20 mmol). The reaction mixture was stirred at room temperature for 1 h to give a gummy precipitate. Methanol was added to dissolve the precipitate, and the reaction mixture was stirred for an additional 1 h. The reaction mixture was concentrated under reduced pressure and partitioned between ethyl acetate and saturated aqueous sodium bicarbonate. The organic layer was dried (MgSO₄) and concentrated to give the title compound as a clear oil (0.31 g), which solidified on standing.

¹H NMR (CDCl₃) δ 1.65 (br s, 1H), 1.7 (m, 2H), 2.1 (m, 2H), 2.75 (m, 2H), 3.1-3.25 (m, 3H), 3.41 (m, 1H), 3.83 (m, 1H), 5.75 (m, 1H), 7.25-7.40 (m, 5H), 7.60 (s, 1H).

### Step C: Preparation of phenyl 4-[4-(4,5-dihydro-5-phenyl-3-isoxazolyl)-2-thiazolyl]-1-piperidinecarboxylate

To a solution of 4-[4-(4,5-dihydro-5-phenyl-3-isoxazolyl)-2-thiazolyl]piperidine (i.e. the product of Step B) (3.3 g, 10 mmol) and triethylamine (2 mL, 14 mmol) in dichloromethane (40 mL) cooled to −5° C., was added a solution of phenyl chloroformate (1.6 g, 10 mmol) in dichloromethane (10 mL) dropwise over 5 minutes. The reaction mixture was stirred at −5° C. for 30 minutes and then allowed to warm to room temperature. After 2 h, the mixture was washed with 1 N hydrochloric acid and brine, dried (MgSO₄) and concentrated under reduced pressure to give the title compound as a white foam (4.3 g). A 1 g sample was crystallized from ethanol (20 mL) to give a white powder (0.81 g) melting at 123-125° C.

¹H NMR (CDCl₃) δ 1.85 (m, 2H), 2.20 (m, 2H), 2.95-3.22 (m, 2H), 3.30 (m, 1H), 3.45 (m, 1H), 3.85 (m, 1H), 4.30-4.50 (m, 2H), 5.75 (m, 1H), 7.15 (m, 2H), 7.22 (m, 1H), 7.25-7.42 (m, 7H), 7.63 (s, 1H).

### Example 2

### Preparation of 4-[4-(4,5-dihydro-5-phenyl-3-isoxazolyl)-2-thiazolyl]-N-phenyl-1-piperidinecarboxamide (Compound 1)

To a solution of 4-[4-(4,5-dihydro-5-phenyl-3-isoxazolyl)-2-thiazolyl]piperidine (i.e. the product of Example 1, Step B) (0.31 g, 1 mmol) in dichloromethane (2 mL) was added phenyl isocyanate (0.12 g, 1 mmol). The reaction mixture was stirred at room temperature for 1 h, diethyl ether (2 mL) was added, and the solution allowed to stand for 3 days. The resulting solid was filtered, dissolved in hot methanol and allowed to cool to room temperature to give colorless crystals (0.30 g). This material was recrystallized from ethanol (5 mL) at 35° C. to give the title compound as a white powder (0.18 g) melting at 140-145° C.

¹H NMR (CDCl₃) δ 1.85 (m, 2H), 2.20 (m, 2H), 3.10 (m, 2H), 3.30 (m, 1H), 3.42 (m, 1H), 3.85 (m, 1H), 4.19 (m, 2H), 5.75 (m, 1H), 6.40 (br s, 1H), 7.05 (m, 1H), 7.22-7.42 (m, 9H), 7.62 (s, 1H).

### Example 3

### Preparation of 4-[4-(4,5-dihydro-5-phenyl-3-isoxazolyl)-2-thiazolyl]-N-(2,5-dimethylphenyl)-1-piperidinecarbothioamide (Compound 75)

To a solution of 4-[4-(4,5-dihydro-5-phenyl-3-isoxazolyl)-2-thiazolyl]piperidine (i.e. the product of Example 1, Step B) (1.0 g, 3.2 mmol) in dichloromethane (10 mL) was added a solution of 2,5-dimethylphenyl isothiocyanate (0.52 g, 3.2 mmol) in dichloromethane (5 mL) over 1 minute. The reaction mixture was stirred at room temperature for 20 minutes, concentrated, dissolved in methyl acetate (4 mL), held at 0° C. overnight and filtered to give the title compound as a white powder (1.35 g) melting at 120-123° C.

¹H NMR (CDCl₃) δ 1.9 (m, 2H), 2.15 (m, 2H), 2.22 (s, 3H), 2.30 (s, 3H), 3.20 (m, 2H), 3.30 (m, 1H), 3.41 (m, 1H), 3.82 (m, 1H), 4.58 (m, 2H), 5.75 (m, 1H), 6.93 (m, 3H), 7.10 (m, 1H), 7.25-7.40 (m, 5H), 7.62 (s, 1H).

### Example 4

### Preparation of 4-[4-(4,5-dihydro-5-phenyl-3-isoxazolyl)-2-thiazolyl]-N-(2,5-dimethylphenyl)-1-piperazinecarboxamide (Compound 70)

### Step A: Preparation of 1,1-dimethylethyl 4-(aminothioxomethyl)-1-piperazine-carboxylate

To a solution of thiocarbonyldiimidazole (2.1 g, 11.8 mmol) in tetrahydrofuran (30 mL) at room temperature, was added 1,1-dimethylethyl 1-piperazinecarboxylate (2.0 g, 10.7 mmol). The reaction mixture was stirred at room temperature for 2 h and then heated to 55° C. for additional 2 h. The reaction mixture was cooled to room temperature and concentrated under reduced pressure until approximately 20 mL of tetrahydrofuran remained. The residue was then treated with a 2 M solution of ammonia in methanol (10 mL) and stirred at room temperature for 24 h. The reaction mixture was concentrated under reduced pressure, and the residue was triturated with diethyl ether (25 mL) to give a white precipitate. The precipitate was filtered and dried to give 1.5 g of the title compound as a white solid.

¹H NMR (CDCl₃) δ 1.39 (s, 9H), 3.32 (m, 4H), 3.73 (m, 4H), 7.49 (br s, 2H).

### Step B: Preparation of 3-chloro-N-hydroxy-2-oxopropanimidoyl chloride

To a solution of 1,3-dichloroacetone (100 g, 0.79 mol) in a 2 M solution of hydrogen chloride in diethyl ether (400 mL) at 15° C. was added t-butyl nitrite (55 g, 0.53 mol) over 10 minutes. The reaction progress was monitored by ¹H NMR to obtain ˜85% conversion with no more than 3% of the bis-nitrosation side product. The reaction mixture was concentrated under reduced pressure to leave a semi-solid, which was then thoroughly rinsed with n-BuCl. The resulting solid was collected under filtration to give a 77 g of the title compound as a white solid. The filtrate was further concentrated under reduced pressure to give a semi-solid residue, which was rinsed with additional n-BuCl. The resulting solid was collected under filtration to give additional 15 g of the title compound as a white solid.

¹H NMR (DMSO-d₆) δ 4.96 (s, 2H), 13.76 (s, 1H).

### Step C: Preparation of 2-chloro-1-(4,5-dihydro-5-phenyl-3-isoxazolyl)ethanone

To a mixture of styrene (6.79 g, 65.3 mmol) and sodium bicarbonate (32.1 g, powder) in acetonitrile (100 mL), 3-chloro-N-hydroxy-2-oxopropanimidoyl chloride (i.e. the product of Step B) (10 g, 64 mmol) was added in 10 portions over 20 minutes. The reaction mixture was then stirred for an additional 1 h and filtered. The filtered solid was rinsed with acetonitrile, and the combined filtrates were concentrated under reduced pressure to leave an oil, which was triturated first with hexanes and then with 1-chlorobutane to give 13.6 g of the title compound as a white solid.

¹H NMR (CDCl₃) δ 3.13 (m, 1H), 3.66 (m, 1H), 4.96 (s, 2H), 5.83 (m, 1H), 7.34-7.44 (m, 5H).

### Step D: Preparation of 1,1-dimethylethyl 4-[4-(4,5-dihydro-5-phenyl-3-isoxazolyl)-2-thiazolyl]-1-piperazineacetate

To a solution of 2-chloro-1-(4,5-dihydro-5-phenyl-3-isoxazolyl)ethanone (i.e. the product of Step C) (0.450 g, 2.018 mmol) and 1,1-dimethylethyl 4-(amino-thioxomethyl)-1-piperazinecarboxylate (i.e. the product of Step A) (0.5 g, 2.04 mmol) in ethanol (10 mL) was added triethylamine (0.204 g, 2.013 mmol), and the reaction mixture was stirred at room temperature for 12 h. The reaction mixture was concentrated under reduced pressure, and the residue was partitioned between ethyl acetate (30 mL) and water (30 mL). The organic layer was separated and washed with brine (25 mL), dried (Na₂SO₄), and concentrated under reduced pressure. The crude residue was purified by column chromatography using 20% ethyl acetate in petroleum ether as eluant to give 700 mg of the title compound as a white solid.

¹H NMR (CDCl₃) δ 1.48 (s, 9H), 3.30 (m, 1H), 3.54 (m, 8H), 3.74 (m, 1H), 5.71 (m, 1H), 6.91 (s, 1H), 7.40-7.29 (m, 5H).

### Step E: Preparation of 1-[4-(4,5-dihydro-5-phenyl-3-isoxazolyl)-2-thiazolyl]-piperazine hydrochloride

To a solution of 1,1-dimethylethyl 4-[4-(4,5-dihydro-5-phenyl-3-isoxazolyl)-2-thiazolyl]-1-piperazineacetate (i.e. the product of Step D) (0.7 g, 1.686 mmol) in diethyl ether (10 mL) was added a 2 M solution of hydrogen chloride in methanol (10 mL) at room temperature. The reaction mixture was stirred at room temperature for 8 h. The resulting white precipitate was filtered and dried to give 500 mg of the title compound as a white solid.

¹H NMR (CDCl₃) δ 3.21 (m, 4H), 3.27 (m, 1H), 3.68 (m, 4H), 3.79 (m, 1H), 5.68 (m, 1H), 7.41-7.29 (m, 6H), 9.49 (br s, 2H).

### Step F: Preparation of 4-[4-(4,5-dihydro-5-phenyl-3-isoxazolyl)-2-thiazolyl]-N-(2,5-dimethylphenyl)-1-piperazinecarboxamide

To a solution of 2,5-dimethylaniline (0.0616 g, 0.510 mmol) in dry THF (10 mL) was added triphosgene (0.0308 g, 0.104 mmol) at room temperature. The mixture was cooled to 0° C., and N,N-diisopropylethylamine (0.129 g, 1.015 mmol) was added dropwise. The mixture was stirred at 0° C. for 3 h. A solution of 1-[4-(4,5-dihydro-5-phenyl-3-isoxazolyl)-2-thiazolyl]piperazine hydrochloride (i.e. the product of Step E) (0.16 g, 0.509 mmol) in tetrahydrofuran was added dropwise at 0° C. and the mixture was then stirred at room temperature for 2 h. The mixture was concentrated in vacuum, and the residue was dissolved in EtOAc (50 mL), washed with water (50 mL) and brine (50 mL), dried over Na₂SO₄ and concentrated under reduced pressure. The crude product was purified by column chromatography (10% MeOH/CHCl₃) to provide the title compound as a white solid (0.17 g).

¹H NMR (CDCl₃) δ 2.22 (s, 3H), 2.31 (s, 3H), 3.36-3.30 (m, 1H), 3.65 (s, 8H), 3.81-3.74 (m, 1H), 5.74-5.69 (m, 1H), 6.12 (s, 1H), 6.88-6.86 (d, 1H), 6.92 (s, 1H), 7.08-7.06 (d, 1H), 7.42-7.32 (m, 6H).

### Example 5

### Preparation of 1-[4-[4-[(5R)-4,5-dihydro-5-phenyl-3-isoxazolyl]-2-thiazolyl]-1-piperidinyl]-N-[2,5-dimethylphenyl]carboxamide (Compound 17)

### Step A: Preparation of 4,5-dihydro-N,N-dimethyl-5-phenyl-3-isoxazolecarboxamide

To a solution of 2-(dimethylamino)-N-hydroxy-2-oxoethanimidoyl chloride (prepared according to the procedure of E. Raleigh, U.S. Pat. No. 3,557,089) (6.0 g, 40 mmol) and styrene (6.0 g, 60 mmol) in toluene (15 mL) was added a solution of potassium hydrogen carbonate (5.0 g, 50 mmol) in water (25 mL) over 1 h while keeping the reaction temperature between 7 and 10° C. The reaction mixture was diluted with 10 mL of toluene and stiffed for an additional 10 minutes. The organic layer was separated and washed with water. The organic layer was concentrated under reduced pressure until no styrene remained to give 8.7 g of the title compound as a light yellow oil. This compound was of sufficient purity to use in subsequent reactions.

¹H NMR (CDCl₃) δ 3.08 (s, 3H), 3.32 (s, 3H), 3.35 (dd, 1H), 3.71 (dd, 1H), 5.65 (dd, 1H), 7.35 (m, 5H).

### Step B: Preparation of 4,5-dihydro-5-phenyl-3-isoxazolecarboxylic acid

To a solution of 4,5-dihydro-N,N-dimethyl-5-phenyl-3-isoxazolecarboxamide (i.e. the product of Example 5, Step A) (60.0 g, 275 mmol) in methanol (300 mL) was added an aqueous sodium hydroxide solution (44 g of 50 wt. % aqueous NaOH in 50 mL of water) dropwise over 30 minutes while maintaining the temperature of the reaction mixture at 45° C. The reaction mixture was allowed to cool to room temperature and stirred overnight. The resulting mixture was concentrated under reduced pressure and treated with 200 mL of water. The pH of the reaction mixture was adjusted using concentrated hydrochloric acid to about 1.0. The crude product was extracted into ethyl acetate (200 mL). The ethyl acetate solution was concentrated under reduced pressure, and the residue was triturated with hexanes. The resulting precipitate was filtered, washed with hexanes (2×20 mL), and dried under vacuum to give 46.5 g of the title compound as a solid.

¹H NMR (CDCl₃) δ 3.25 (dd, 1H), 3.75 (dd, 1H), 5.85 (dd, 1H), 7.35 (m, 5H), 8.1 (br s, 1H).

### Step C: Preparation of the Cinchonine Salt of (5R)-4,5-dihydro-5-phenyl-3-isoxazole-carboxylic Acid

A mixture of racemic 4,5-dihydro-5-phenyl-3-isoxazolecarboxylic acid (i.e. the product of Example 5, Step B) (9.5 g, 50 mmol) in methanol (70 mL) was heated to 55° C., and cinchonine (containing about 15% dihydrocinchonine, 14.5 g, 50 mmol) was added over 20 minutes while keeping the temperature of the reaction mixture between 53 and 57° C. The reaction mixture was allowed to cool to room temperature over 60 minutes, and then water (35 mL) was added dropwise over 30 minutes. The resulting slurry was cooled to 10° C. and filtered. The filter cake was washed twice with 10 mL of 25% methanol in water, and air dried to give 8.52 g of the title compound as a solid. The diastereomeric ratio of the product was determined using chiral high performance liquid chromatography (HPLC) analysis on a Daicel Chiralcel® OD HPLC column to be about 99:1.

¹H NMR (CDCl₃) δ 3.25 (dd, 1H), 3.75 (dd, 1H), 5.85 (dd, 1H), 7.35 (m, 5H), 8.1 (br s, 1H).

### Step D: Preparation of (5R)-4,5-dihydro-N,N-dimethyl-5-phenyl-3-isoxazole-carboxamide

The cinchonine salt of (5R)-4,5-dihydro-5-phenyl-3-isoxazolecarboxylic acid (i.e. the product of Example 5, Step C) (98% diastereomeric excess, 16.5 g, 34.3 mmol) was slurried in a mixture of 1 N hydrochloric acid (90 mL), cyclohexane (100 mL) and ethyl acetate (40 mL). After all the solids dissolved, the phases were separated, and the organic layer was washed with brine (20 mL) and concentrated under reduced pressure to give 5.6 g of white solid. To a solution of the resulting free acid (5.0 g, 26.2 mmol) in ethyl acetate (100 mL) at room temperature was added N,N-dimethylformamide (1 drop) followed by thionyl chloride (4.25 g, 35.7 mmol). The reaction mixture was then heated under reflux for 3 h. The resulting mixture was cooled and concentrated under reduced pressure. The residue containing crude acid chloride was dissolved in ethyl acetate (25 mL), and this solution was added in portions to a pre-cooled (5° C.) mixture of dimethylamine in tetrahydrofuran (29 mL of a 2.0 M solution), while maintaining the temperature of the mixture at 5-10° C. When the addition was complete, the reaction mixture was concentrated under reduced pressure, and diluted with water (50 mL). The resulting precipitate was filtered, washed with water and suction-dried overnight to give 4.1 g of the title compound as a light tan solid, melting at 59-61° C. This compound was of sufficient purity to use in subsequent reactions.

### Step E: Preparation of 2-bromo-1-[(5R)-4,5-dihydro-5-phenyl-3-isoxazolyl]ethanone

A solution of (5R)-4,5-dihydro-N,N-dimethyl-5-phenyl-3-isoxazolecarboxamide (i.e. the product of Example 5, Step D) (3.5 g, 16.0 mmol) in a mixture of tetrahydrofuran (5 mL) and toluene (10 mL) was cooled to −15° C., and methyl magnesium bromide (3.0 M solution in tetrahydrofuran, 8.8 mL, 26.4 mmol) was added over 1 h at −15° C. Then the reaction mixture was poured over a mixture of 20 g of concentrated hydrochloric acid and 80 g of ice, and the organic phase was separated. The aqueous phase was extracted with ethyl acetate (100 mL), and the combined extract was washed with brine (40 mL) and concentrated under reduced pressure to give 3.2 g of 1-[(5R)-4,5-dihydro-5-phenyl-3-isoxazoyl]ethanone.

¹H NMR (CDCl₃) δ 2.55 (s, 3H), 3.17 (dd, 1H), 3.54 (dd, 1H), 5.75 (dd, 1H), 7.35 (m, 5H).

1-[(5R)-4,5-dihydro-5-phenyl-3-isoxazoyl]ethanone (3.2 g, 16.7 mmol) was dissolved in 1,2-dichloroethane (15 mL), and a solution of bromine (2.13 g, 13.3 mmol) in dichloroethane (5 mL) was added over 30 minutes while maintaining the temperature of the reaction mixture at about 30° C. The reaction mixture was diluted with water (10 mL), and the organic layer was concentrated under reduced pressure and purified by medium-pressure liquid chromatography using 35% of dichloromethane in hexanes as eluant to give 2.6 g of the title compound as a white solid, melting at 31-33° C.

¹H NMR (CDCl₃): δ 3.20 (dd, 1H), 3.60 (dd, 1H), 4.49 (s, 2H), 5.80 (dd, 1H), 7.35 (m, 5H).

### Step F: Preparation of 4-cyano-N-(2,5-dimethylphenyl)piperidinecarboxamide

A solution of 4-cyanopiperidine (11.0 g, 100 mmol) in diethyl ether (350 mL) was cooled to 0° C. with an ice-water bath. A solution of 2,5-dimethylphenyl isocyanate (14.7 g, 100 mmol) in diethyl ether (50 mL) was added into the reaction mixture over 30 minutes to give a thick precipitate. The reaction mixture was warmed to room temperature, and the resulting solids were filtered, washed with diethyl ether and air-dried to give 25.3 g of the title compound as a white powder, melting at 187-190° C.

¹H NMR (CDCl₃) δ 1.95 (m, 4H), 2.19 (s, 3H), 2.30 (s, 3H), 2.90 (m, 1H), 3.45 (m, 2H), 3.70 (m, 2H), 6.10 (br s, 1H), 6.85 (m, 1H), 7.04 (m, 1H), 7.37 (m, 1H).

### Step G: Preparation of N-(2,5-dimethylphenyl)-4-thiocarbamoylpiperidine-carboxamide

A mixture of 4-cyano-N-(2,5-dimethylphenyl)piperidinecarboxamide (i.e. the product of Step F) (12.75 g, 49.6 mmol), sodium hydrosulfide hydrate (11.1 g, 150 mmol) and diethylamine hydrochloride (10.9 g, 100 mmol) in N,N-dimethylformamide (50 mL) was stirred at room temperature for 3 days. The resulting thick, green suspension was added dropwise into ice water (600 mL). The resulting solid was filtered, washed with water and air-dried to give 12.5 g of the title compound as a tan solid decomposing at 155-156° C.

¹H NMR (DMSO-d₆) δ 1.67 (m, 4H), 2.10 (s, 3H), 2.23 (s, 3H), 2.75 (m, 3H), 4.15 (m, 2H), 6.85 (m, 1H), 7.0 (m, 1H), 7.05 (m, 1H), 7.95 (br s, 1H), 9.15 (br s, 1H), 9.22 (br s, 1H).

### Step H: Preparation of 1-[4-[4-[(5R)-4,5-dihydro-5-phenyl-3-isoxazolyl]-2-thiazolyl]-1-piperidinyl]-N-[2,5-dimethylphenyl]carboxamide

A mixture of N-(2,5-dimethylphenyl)-4-thiocarbamoylpiperidinecarboxamide (i.e. the product of Step B) (291 mg, 1.0 mmol) and 2-bromo-1-[(5R)-4,5-dihydro-5-phenyl-3-isoxazolyl]ethanone (i.e. the product of Example 5, Step E) (268 mg, 1.0 mmol) in acetone (10 mL) was vortexed (VWR Mini-Vortexer) for 16 h and then heated at 45° C. for 1 h. The reaction mixture was allowed to cool to room temperature, treated with solid sodium bicarbonate (168 mg, 2.0 mmol), and stiffed for 1 h. The reaction mixture was then concentrated under reduced pressure, diluted with ethyl acetate, washed with water and brine, dried (MgSO₄), and concentrated under reduced pressure to give the title product as a pale-yellow foam. The sample was dissolved in methyl acetate (2 mL) and allowed to sit at room temperature and then at 0° C. to give 220 mg of colorless crystals melting at 120-125° C. A second preparation was crystallized from methanol to give large prisms melting at 121-124° C.

¹H NMR (CDCl₃) δ 1.85 (m, 2H), 1.99 (m, 2H), 2.21 (s, 3H), 2.31 (s, 3H), 3.08 (m, 2H), 3.25 (m, 1H), 3.42 (dd, 1H), 3.82 (dd, 1H), 4.15 (m, 2H), 5.78 (dd, 1H), 6.12 (br s, 1H), 6.82 (m, 1H), 7.02 (m, 1H), 7.2-7.4 (m, 5H), 7.46 (m, 1H), 7.62 (s, 1H).

By the procedures described herein together with methods known in the art, the following compounds of Tables 1A to 4C can be prepared. The following abbreviations are used in the Tables which follow: i means iso, Me means methyl, Et means ethyl, Pr means propyl, i-Pr means isopropyl, Bu means butyl, Ph means phenyl, OMe means methoxy, —CN means cyano and S(O)₂Me means methylsulfonyl.

TABLE 1A

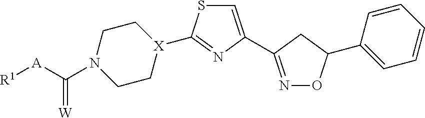

| R¹ | A | W |
|---|---|---|
| phenyl | O | O |
| 2-methylphenyl | O | O |
| 3-methylphenyl | O | O |
| 4-methylphenyl | O | O |
| 2-fluorophenyl | O | O |
| 3-fluorophenyl | O | O |
| 4-fluorophenyl | O | O |
| 2-chlorophenyl | O | O |
| 3-chlorophenyl | O | O |
| 4-chlorophenyl | O | O |
| 2-bromophenyl | O | O |
| 3 -bromophenyl | O | O |
| 4-bromophenyl | O | O |
| 2-iodophenyl | O | O |
| 3-iodophenyl | O | O |
| 4-iodophenyl | O | O |
| 2-methoxyphenyl | O | O |
| 3 -methoxyphenyl | O | O |
| 4-methoxyphenyl | O | O |
| 2-trifluoromethoxyphenyl | O | O |
| 3 -trifluoromethoxyphenyl | O | O |
| 4-trifluoromethoxyphenyl | O | O |
| 2-trifluoromethylphenyl | O | O |
| 3 -trifluoromethylphenyl | O | O |
| 4-trifluoromethylphenyl | O | O |
| 2-cyanophenyl | O | O |
| 3-cyanophenyl | O | O |
| 4-cyanophenyl | O | O |
| 4-hydroxyphenyl | O | O |
| 4-aminophenyl | O | O |
| 3 -nitrophenyl | O | O |
| 2-ethylphenyl | O | O |
| 4-t-butylphenyl | O | O |
| 4-cyclopropylphenyl | O | O |
| 2-methoxymethylphenyl | O | O |
| 4-(methoxyethyl)phenyl | O | O |
| 4-methylthiophenyl | O | O |
| 4-trifluoromethylthiophenyl | O | O |
| 4-methylsulfinylphenyl | O | O |
| 4-methylsulfonylphenyl | O | O |
| 4-isopropylaminophenyl | O | O |
| 4-dimethylaminophenyl | O | O |
| 4-hydroxymethylphenyl | O | O |
| 4-methylcarbonylphenyl | O | O |
| 2-methoxycarbonylphenyl | O | O |
| 2-methylaminocarbonylphenyl | O | O |
| 2-dimethylaminocarbonylphenl | O | O |
| 4-t-butylcarbonyloxyphenyl | O | O |
| 4-methylcarbonylthiophenyl | O | O |
| 4-trimethylsilylphenyl | O | O |
| 2,3-dichlorophenyl | O | O |
| 2,4-dichlorophenyl | O | O |
| 2,5 -dichlorophenyl | O | O |
| 2,6-dichlorophenyl | O | O |
| 2,3 -dimethylphenyl | O | O |
| 2,4-dimethylphenyl | O | O |
| 2,5 -dimethylphenyl | O | O |
| 2,6-dimethylphenyl | O | O |
| 2,4,6-trimethylphenyl | O | O |
| 2-chloro-4-methylphenyl | O | O |
| pyridin-2-yl | O | O |
| pyridin-3-yl | O | O |
| pyridin-4-yl | O | O |
| 3-chloropyridin-2-yl | O | O |
| 4-chloropyridin-2-yl | O | O |
| 5-chloropyridin-2-yl | O | O |
| 6-chloropyridin-2-yl | O | O |
| 5-bromopyridin-3-yl | O | O |
| 3-chloro-5-trifluoromethylpyridin-2-yl | O | O |
| pyrimidin-2-yl | O | O |
| 4,6-dimethylpyrimidin-2-yl | O | O |
| 6-methylpyrimidin-4-yl | O | O |
| 2-thienyl | O | O |
| 3-thienyl | O | O |
| oxazol-2-yl | O | O |
| thiazol-2-yl | O | O |
| 1,3-dimethylpyrazol-5-yl | O | O |
| 3-methyl-1,2,4-triazol-5-yl | O | O |
| 1-methylimidazol-2-yl | O | O |
| 2-methyl-1,3,4-oxadiazol-5-yl | O | O |
| 2-methyl-1,3,4-thiadiazol-5-yl | O | O |
| 1-naphthalenyl | O | O |
| 2-naphthalenyl | O | O |
| 1,2-benzoisoxazol-3-yl | O | O |
| phenyl | NH | O |
| 2-methylphenyl | NH | O |
| 3-methylphenyl | NH | O |
| 4-methylphenyl | NH | O |
| 2-fluorophenyl | NH | O |
| 3-fluorophenyl | NH | O |
| 4-fluorophenyl | NH | O |
| 2-chlorophenyl | NH | O |
| 3-chlorophenyl | NH | O |
| 4-chlorophenyl | NH | O |
| 2-bromophenyl | NH | O |
| 3-bromophenyl | NH | O |
| 4-bromophenyl | NH | O |
| 2-iodophenyl | NH | O |
| 3-iodophenyl | NH | O |
| 4-iodophenyl | NH | O |
| 2-methoxyphenyl | NH | O |
| 3-methoxyphenyl | NH | O |
| 4-methoxyphenyl | NH | O |
| 2-trifluoromethoxyphenyl | NH | O |
| 3-trifluoromethoxyphenyl | NH | O |
| 4-trifluoromethoxyphenyl | NH | O |
| 2-trifluoromethylphenyl | NH | O |
| 3-trifluoromethylphenyl | NH | O |
| 4-trifluoromethylphenyl | NH | O |
| 2-cyanophenyl | NH | O |
| 3-cyanophenyl | NH | O |
| 4-cyanophenyl | NH | O |
| 4-hydroxyphenyl | NH | O |
| 4-aminophenyl | NH | O |
| 3-nitrophenyl | NH | O |
| 2-ethylphenyl | NH | O |
| 4-t-butylphenyl | NH | O |
| 4-cyclopropylphenyl | NH | O |
| 2-methoxymethylphenyl | NH | O |
| 4-(methoxyethyl)phenyl | NH | O |
| 4-methylthiophenyl | NH | O |
| 4-trifluoromethylthiophenyl | NH | O |
| 4-methylsulfinylphenyl | NH | O |
| 4-methylsulfonylphenyl | NH | O |
| 4-isopropylaminophenyl | NH | O |
| 4-dimethylaminophenyl | NH | O |
| 4-hydroxymethylphenyl | NH | O |
| 4-methylcarbonylphenyl | NH | O |
| 2-methoxycarbonylphenyl | NH | O |
| 2-methylaminocarbonylphenyl | NH | O |
| 2-dimethylaminocarbonylphenyl | NH | O |
| 4-t-butylcarbonyloxyphenyl | NH | O |
| 4-methylcarbonylthiophenyl | NH | O |
| 4-trimethylsilylphenyl | NH | O |
| 2,3-dichlorophenyl | NH | O |
| 2,4-dichlorophenyl | NH | O |
| 2,5-dichlorophenyl | NH | O |
| 2,6-dichlorophenyl | NH | O |
| 2,3-dimethylphenyl | NH | O |
| 2,4-dimethylphenyl | NH | O |
| 2,5-dimethylphenyl | NH | O |
| 2,6-dimethylphenyl | NH | O |
| 2,4,6-trimethylphenyl | NH | O |
| 2-chloro-4-methylphenyl | NH | O |
| pyridin-2-yl | NH | O |
| pyridin-3-yl | NH | O |
| pyridin-4-yl | NH | O |
| 3-chloropyridin-2-yl | NH | O |
| 4-chloropyridin-2-yl | NH | O |
| 5-chloropyridin-2-yl | NH | O |
| 6-chloropyridin-2-yl | NH | O |
| 5-bromopyridin-3-yl | NH | O |
| 3-chloro-5-trifluoromethylpyridin-2-yl | NH | O |
| pyrimidin-2-yl | NH | O |
| 4,6-dimethylpyrimidin-2-yl | NH | O |
| 6-methylpyrimidin-4-yl | NH | O |
| 2-thienyl | NH | O |
| 3-thienyl | NH | O |
| oxazol-2-yl | NH | O |
| thiazol-2-yl | NH | O |
| 1,3-dimethylpyrazol-5-yl | NH | O |
| 3-methyl-1,2,4-triazol-5-yl | NH | O |
| 1-methylimidazol-2-yl | NH | O |
| 2-methyl-1,3,4 -oxadiazol-5-yl | NH | O |
| 2-methyl-1,3,4-thiadiazol-5-yl | NH | O |
| 1-naphthalenyl | NH | O |
| 2-naphthalenyl | NH | O |
| 1,2-benzoisoxazol-3-yl | NH | O |
| phenyl | S | O |
| 2-methylphenyl | S | O |
| 3-methylphenyl | S | O |
| 4-methylphenyl | S | O |
| 2-fluorophenyl | S | O |
| 3-fluorophenyl | S | O |
| 4-fluorophenyl | S | O |
| 2-chlorophenyl | S | O |
| 3-chlorophenyl | S | O |
| 4-chlorophenyl | S | O |
| 2-bromophenyl | S | O |
| 3-bromophenyl | S | O |
| 4-bromophenyl | S | O |
| 2-iodophenyl | S | O |
| 3-iodophenyl | S | O |
| 4-iodophenyl | S | O |
| 2-methoxyphenyl | S | O |
| 3-methoxyphenyl | S | O |
| 4-methoxyphenyl | S | O |
| 2-trifluoromethoxyphenyl | S | O |
| 3-trifluoromethoxyphenyl | S | O |
| 4-trifluoromethoxyphenyl | S | O |
| 2-trifluoromethylphenyl | S | O |
| 3-trifluoromethylphenyl | S | O |
| 4-trifluoromethylphenyl | S | O |
| phenyl | O | S |
| 2-methylphenyl | O | S |
| 3-methylphenyl | O | S |
| 4-methylphenyl | O | S |
| 2-fluorophenyl | O | S |
| 3-fluorophenyl | O | S |
| 4-fluorophenyl | O | S |
| 2-chlorophenyl | O | S |
| 3-chlorophenyl | O | S |
| 4-chlorophenyl | O | S |
| 2-bromophenyl | O | S |
| 3-bromophenyl | O | S |
| 4-bromophenyl | O | S |
| 2-iodophenyl | O | S |
| 3-iodophenyl | O | S |
| 4-iodophenyl | O | S |
| 2-methoxyphenyl | O | S |
| 3-methoxyphenyl | O | S |
| 4-methoxyphenyl | O | S |
| 2-trifluoromethoxyphenyl | O | S |
| 3-trifluoromethoxyphenyl | O | S |
| 4-trifluoromethoxyphenyl | O | S |
| 2-trifluoromethylphenyl | O | S |
| 3-trifluoromethylphenyl | O | S |
| 4-trifluoromethylphenyl | O | S |
| phenyl | NH | S |
| 2-methylphenyl | NH | S |
| 3-methylphenyl | NH | S |
| 4-methylphenyl | NH | S |
| 2-fluorophenyl | NH | S |
| 3-fluorophenyl | NH | S |
| 4-fluorophenyl | NH | S |
| 2-chlorophenyl | NH | S |
| 3-chlorophenyl | NH | S |
| 4-chlorophenyl | NH | S |
| 2-bromophenyl | NH | S |
| 3-bromophenyl | NH | S |
| 4-bromophenyl | NH | S |
| 2-iodophenyl | NH | S |
| 3-iodophenyl | NH | S |
| 4-iodophenyl | NH | S |
| 2-methoxyphenyl | NH | S |
| 3-methoxyphenyl | NH | S |
| 4-methoxyphenyl | NH | S |
| 2-trifluoromethoxyphenyl | NH | S |
| 3-trifluoromethoxyphenyl | NH | S |
| 4-trifluoromethoxyphenyl | NH | S |
| 2-trifluoromethylphenyl | NH | S |
| 3 -trifluoromethylphenyl | NH | S |
| 4 -trifluoromethylphenyl | NH | S |
| phenyl | S | S |
| phenyl | N—Me | O |
| phenyl | N—Et | O |
| phenyl | N—Pr | O |
| phenyl | N—iBu | O |
| phenyl | NCH₂CH═CH₂ | O |
| phenyl | N—CH₂C≡CH | O |
| phenyl | N—CH₂CF₃ | O |
| phenyl | N—CH₂CH₂OMe | O |
| phenyl | N—(CO)Me | O |
| phenyl | N—(CO)CF₃ | O |
| phenyl | N—SO₂Me | O |
| phenyl | N—SO₂CF₃ | O |

X is CH.

### Table 1B

Table 1B is constructed the same as Table 1A, except that X is N.

TABLE 2A

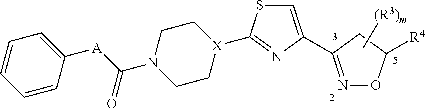

| (R³)ₘ | R⁴ | (R³)ₘ | R⁴ |
|---|---|---|---|
| — | phenyl | — | 4-trimethylsilylphenyl |
| — | 2-methylphenyl | — | 2,3-dichlorophenyl |
| — | 3-methylphenyl | — | 2,4-dichlorophenyl |
| — | 4-methylphenyl | — | 2,5-dichlorophenyl |
| — | 2-fluorophenyl | — | 2,6-dichlorophenyl |
| — | 3-fluorophenyl | — | 2,3-dimehylphenyl |
| — | 4-fluorophenyl | — | 2,4-dimethylphenyl |
| — | 2-chlorophenyl | — | 2,5-dimethylphenyl |
| — | 3-chlorophenyl | — | 2,6-dimthylphenyl |
| — | 4-chlorophenyl | — | 2,4,6-trimethylphenyl |
| — | 2-bromophenyl | — | 2-chloro-4-methylphenyl |
| — | 3-bromophenyl | — | pyridin-2-yl |
| — | 4-bromophenyl | — | pyridin-3-yl |
| — | 2-iodophenyl | — | pyridin-4-yl |
| — | 3-iodophenyl | — | 2-thienyl |
| — | 4-iodophenyl | — | 3-thienyl |
| — | 2-methoxyphenyl | — | oxazol-2-yl |
| — | 3-methoxyphenyl | — | thiazol-2-yl |
| — | 4-methoxyphenyl | — | imidazol-1-yl |
| — | 2-trifluoromethoxyphenyl | — | 1-methylimidazol-4-yl |
| — | 3-trifluoromethoxyphenyl | — | 1,2,4-triazol-1-yl |
| — | 4-trifluoromethoxyphenyl | — | 3,5-dimethylpyrazol-1-yl |
| — | 2-trifluoromethylphenyl | — | 1-naphthalenyl |
| — | 3-trifluoromethylphenyl | — | 2-naphthalenyl |
| — | 4-trifluoromethylphenyl | — | benzyl |
| — | 2-cyanophenyl | 5-methyl | phenyl |
| — | 3 -cyanophenyl | 5-ethyl | phenyl |
| — | 4 -cyanophenyl | 5-propyl | phenyl |
| — | 4 -hydroxyphenyl | 5-trifluoromethyl | phenyl |
| — | 4 -aminophenyl | 4-cyano | phenyl |
| — | 3 -nitrophenyl | 4,4-dimethyl | phenyl |
| — | 2-ethylphenyl | 5-methyl | methyl |
| — | 4 -t-butylphenyl | — | n-octyl |
| — | 4 -cyc lopropylphenyl | — | trifluoromethyl |
| — | 2-methoxymethylphenyl | — | cyclohexyl |
| — | 4 -(methoxyethyl)phenyl | — | cyclopropyl |
| — | 4-methylthiophenyl | — | methoxymethyl |
| — | 4-trifluoromethylthiophenyl | — | ethoxymethyl |
| — | 4-methylsulfinylphenyl | — | methylthiomethyl |
| — | 4-methylsulfonylphenyl | — | methylsulfinylmethyl |
| — | 4-isopropylaminophenyl | — | methylsulfonylmethyl |
| — | 4-dimethylaminophenyl | — | methylaminomethyl |
| — | 4-hydroxymethylphenyl | — | dimethylaminomethyl |
| — | 4-methylcarbonylphenyl | — | diethylaminomethly |
| — | 2-methoxycarbonylphenyl | — | methylcarbonyl |
| — | 2-methylaminocarbonylphenyl | — | trifluoromethylcarbonyl |
| — | 2-dimethylaminocarbonylphenyl | — | methoxycarbonyl |
| — | 4-t-butylcarbonyloxyphenyl | — | hexylaminocarbonyl |
| — | 4-methylcarbonylthiophenyl | — | dipropylaminocarbonyl |

A dash “—” in the (R³)ₘ column means no R³ substituent is present.
A is O; X is CH.

### Table 2B

Table 2B is constructed the same as Table 2A, except that A is NH and X is CH.

### Table 2C

Table 2C is constructed the same as Table 2A, except that A is S and X is CH.

### Table 2D

Table 2D is constructed the same as Table 2A, except that A is O and X is N.

### Table 2E

Table 2E is constructed the same as Table 2A, except that A is NH and X is N.

### Table 2F

Table 2F is constructed the same as Table 2A, except that A is S and X is N.

TABLE 3A

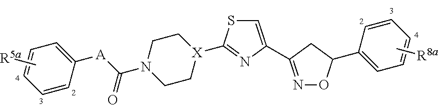

| R^{5a} | R^{8a} | R^{5a} | R^{8a} |
|---|---|---|---|
| 3-methyl | 2-methyl | 3-methyl | 4-chloro |
| 4-methyl | 2-methyl | 4-methyl | 4-chloro |
| 2-fluoro | 2-methyl | 2-fluoro | 4-chloro |
| 3-fluoro | 2-methyl | 3-fluoro | 4-chloro |
| 4-fluoro | 2-methyl | 4-fluoro | 4-chloro |
| 2-chloro | 2-methyl | 2-chloro | 4-chloro |
| 3-chloro | 2-methyl | 3-chloro | 4-chloro |
| 4-chloro | 2-methyl | 4-chloro | 4-chloro |
| 2-bromo | 2-methyl | 2-bromo | 4-chloro |
| 3-bromo | 2-methyl | 3-bromo | 4-chloro |
| 4-bromo | 2-methyl | 4-bromo | 4-chloro |
| 2-iodo | 2-methyl | 2-iodo | 4-chloro |
| 3-iodo | 2-methyl | 3-iodo | 4-chloro |
| 4-iodo | 2-methyl | 4-iodo | 4-chloro |
| 2-methoxy | 2-methyl | 2-methoxy | 4-chloro |
| 3-methoxy | 2-methyl | 3-methoxy | 4-chloro |
| 4-methoxy | 2-methyl | 4-methoxy | 4-chloro |
| 2-trifluoromethoxy | 2-methyl | 2-trifluoromethoxy | 4-chloro |
| 3-trifluoromethoxy | 2-methyl | 3-trifluoromethoxy | 4-chloro |
| 4-trifluoromethoxy | 2-methyl | 4-trifluoromethoxy | 4-chloro |
| 2-trifluoromethyl | 2-methyl | 2-trifluoromethyl | 4-chloro |
| 3-trifluoromethyl | 2-methyl | 3-trifluoromethyl | 4-chloro |
| 4-trifluoromethyl | 2-methyl | 4-trifluoromethyl | 4-chloro |
| 2-cyano | 2-methyl | 2-cyano | 4-chloro |
| 3-cyano | 2-methyl | 3-cyano | 4-chloro |
| 4-cyano | 2-methyl | 4-cyano | 4-chloro |
| 2-methyl | 4-methyl | 2-methyl | 2,6-dimethyl |
| 3-methyl | 4-methyl | 3-methyl | 2,6-dimethyl |
| 4-methyl | 4-methyl | 4-methyl | 2,6-dimethyl |
| 2-fluoro | 4-methyl | 2-fluoro | 2,6-dimethyl |
| 3-fluoro | 4-methyl | 3-fluoro | 2,6-dimethyl |
| 4-fluoro | 4-methyl | 4-fluoro | 2,6-dimethyl |
| 2-chloro | 4-methyl | 2-chloro | 2,6-dimethyl |
| 3-chloro | 4-methyl | 3-chloro | 2,6-dimethyl |
| 4-chloro | 4-methyl | 4-chloro | 2,6-dimethyl |
| 2-bromo | 4-methyl | 2-bromo | 2,6-dimethyl |
| 3-bromo | 4-methyl | 3-bromo | 2,6-dimethyl |
| 4-bromo | 4-methyl | 4-bromo | 2,6-dimethyl |
| 2-iodo | 4-methyl | 2-iodo | 2,6-dimethyl |
| 3-iodo | 4-methyl | 3-iodo | 2,6-dimethyl |
| 4-iodo | 4-methyl | 4-iodo | 2,6-dimethyl |
| 2-methoxy | 4-methyl | 2-methoxy | 2,6-dimethyl |
| 3-methoxy | 4-methyl | 3-methoxy | 2,6-dimethyl |
| 4-methoxy | 4-methyl | 4-methoxy | 2,6-dimethyl |
| 2-trifluoromethoxy | 4-methyl | 2-trifluoromethoxy | 2,6-dimethyl |
| 3-trifluoromethoxy | 4-methyl | 3-trifluoromethoxy | 2,6-dimethyl |
| 4-trifluoromethoxy | 4-methyl | 4-trifluoromethoxy | 2,6-dimethyl |
| 2-trifluoromethyl | 4-methyl | 2-trifluoromethyl | 2,6-dimethyl |
| 3-trifluoromethyl | 4-methyl | 3 -trifluoromethyl | 2,6-dimethyl |
| 4-trifluoromethyl | 4-methyl | 4-trifluoromethyl | 2,6-dimethyl |
| 2-cyano | 4-methyl | 2-cyano | 2,6-dimethyl |
| 3-cyano | 4-methyl | 3-cyano | 2,6-dimethyl |
| 4-cyano | 4-methyl | 4-cyano | 2,6-dimethyl |
| 2-methyl | 2-chloro | 2-methyl | 2,6-difluoro |
| 3-methyl | 2-chloro | 3-methyl | 2,6-difluoro |
| 4-methyl | 2-chloro | 4-methyl | 2,6-difluoro |
| 2-fluoro | 2-chloro | 2-fluoro | 2,6-difluoro |
| 3-fluoro | 2-chloro | 3-fluoro | 2,6-difluoro |
| 4-fluoro | 2-chloro | 4-fluoro | 2,6-difluoro |
| 2-chloro | 2-chloro | 2-chloro | 2,6-difluoro |
| 3-chloro | 2-chloro | 3-chloro | 2,6-difluoro |
| 4-chloro | 2-chloro | 4-chloro | 2,6-difluoro |
| 2-bromo | 2-chloro | 2-bromo | 2,6-difluoro |
| 3-bromo | 2-chloro | 3-bromo | 2,6-difluoro |
| 4-bromo | 2-chloro | 4-bromo | 2,6-difluoro |
| 2-iodo | 2-chloro | 2-iodo | 2,6-difluoro |
| 3-iodo | 2-chloro | 3 -iodo | 2,6-difluoro |
| 4-iodo | 2-chloro | 4-iodo | 2,6-difluoro |
| 2-methoxy | 2-chloro | 2-methoxy | 2,6-difluoro |
| 3-methoxy | 2-chloro | 3 -methoxy | 2,6-difluoro |
| 4-methoxy | 2-chloro | 4-methoxy | 2,6-difluoro |
| 2-trifluoromethoxy | 2-chloro | 2-trifluoromethoxy | 2,6-difluoro |
| 3-trifluoromethoxy | 2-chloro | 3-trifluoromethoxy | 2,6-difluoro |
| 4-trifluoromethoxy | 2-chloro | 4-trifluoromethoxy | 2,6-difluoro |
| 2-trifluoromethyl | 2-chloro | 2-trifluoromethyl | 2,6-difluoro |
| 3-trifluoromethyl | 2-chloro | 3 -trifluoromethyl | 2,6-difluoro |
| 4-trifluoromethyl | 2-chloro | 4-trifluoromethyl | 2,6-difluoro |
| 2-cyano | 2-chloro | 2-cyano | 2,6-difluoro |
| 3-cyano | 2-chloro | 3-cyano | 2,6-difluoro |
| 4-cyano | 2-chloro | 4-cyano | 2,6-difluoro |

A is O; X is CH.

### Table 3B

Table 3B is constructed the same as Table 3A, except that A is NH and X is CH.

### Table 3C

Table 3C is constructed the same as Table 3A, except that A is S and X is CH.

### Table 3D

Table 3D is constructed the same as Table 3A, except that A is O and X is N.

### Table 3E

Table 3E is constructed the same as Table 3A, except that A is NH and X is N.

### Table 3F

Table 3F is constructed the same as Table 3A, except that A is S and X is N.

TABLE 4A

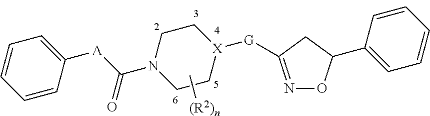

| (R²)ₙ | X | G | (R^{Y})_{q} |
|---|---|---|---|
| — | CH | G-1 | — |
| — | CH | G-2 | — |
| — | CH | G-3 | — |
| — | CH | G-4 | — |
| — | CH | G-5 | — |
| — | CH | G-6 | — |
| — | CH | G-7 | 1-Me |
| — | CH | G-8 | 1-Me |
| — | CH | G-9 | 1-H |
| — | CH | G-10 | — |
| — | CH | G-11 | — |
| — | CH | G-121 | 1-Me |
| — | CH | G-13 | 1-H |
| — | CH | G-14 | — |
| — | CH | G-15 | — |
| — | CH | G-16 | — |
| — | CH | G-17 | — |
| — | CH | G-18 | — |
| — | CH | G-19 | 1-H |
| — | CH | G-20 | 1-Me |
| — | CH | G-21 | — |
| — | CH | G-22 | 1-H |
| — | CH | G-23 | 1-H |
| — | CH | G-24 | — |
| — | CH | G-25 | — |
| — | CH | G-26 | — |
| — | CH | G-27 | — |
| — | CH | G-28 | — |
| — | CH | G-29 | — |
| — | CH | G-30 | — |
| — | CH | G-31 | — |
| — | CH | G-32 | — |
| — | CH | G-33 | — |
| — | CH | G-34 | — |
| — | CH | G-35 | — |
| — | CH | G-36 | 1-Me |
| — | CH | G-37 | — |
| — | CH | G-38 | — |
| — | CH | G-39 | — |
| — | CH | G-40 | 1-H |
| — | CH | G-41 | — |
| — | CH | G-42 | 1-H |
| — | CH | G-43 | — |
| — | CH | G-44 | — |
| — | CH | G-45 | — |
| — | CH | G-46 | — |
| — | CH | G-47 | — |
| — | CH | G-48 | — |
| — | N | G-1 | — |
| — | N | G-2 | — |
| — | N | G-3 | — |
| — | N | G-4 | — |
| — | N | G-5 | — |
| — | N | G-6 | — |
| — | N | G-7 | 1-Me |
| — | N | G-8 | 1-Me |
| — | N | G-9 | 1-H |
| — | N | G-10 | — |
| — | N | G-12 | 1-Me |
| — | N | G-13 | 1-H |
| — | N | G-14 | — |
| — | N | G-17 | — |
| — | N | G-19 | 1-H |
| — | N | G-20 | 1-Me |
| — | N | G-21 | — |
| — | N | G-22 | 1-H |
| — | N | G-23 | 1-H |
| — | N | G-24 | — |
| — | CH | G-25 | — |
| — | CH | G-26 | — |
| — | CH | G-27 | — |
| — | CH | G-28 | — |
| — | CH | G-29 | — |
| — | CH | G-30 | — |
| — | N | G-31 | — |
| — | N | G-32 | — |
| — | N | G-33 | — |
| — | N | G-34 | — |
| — | N | G-35 | — |
| — | CH | G-36 | 1-Me |
| — | N | G-39 | — |
| — | N | G-40 | 1-H |
| — | N | G-42 | 1-H |
| — | N | G-43 | — |
| — | N | G-44 | — |
| — | N | G-45 | — |
| — | N | G-46 | — |
| — | N | G-47 | — |
| — | N | G-48 | — |
| — | CC1 | G-26 | — |
| — | CCN | G-26 | — |
| — | COH | G-26 | — |
| 3-OH | CH | G-26 | — |
| 2-Me | N | G-26 | — |
| 3-Me | N | G-26 | — |
| 2-Et | CH | G-26 | — |
| — | CCF3 | G-26 | — |
| — | COMB | G-26 | — |
| 2,6-Bible | N | G-26 | 5-Me |
| — | CH | G-26 | 5-Cl |
| — | CH | G-26 | 5-Br |
| — | CH | G-26 | 5-CN |
| — | CH | G-26 | 5-CF₃ |
| — | CH | G-26 | 5-Me |
| — | CH | G-34 | — |

A is O.
A dash “—” in the (R²)ₙ column means no R² substituent is present on ring members other than X.
The entries in the columns headed by G and (R^{Y})_{q} refer to groups defined in Exhibit 2.
A dash “—” in the (R^{Y})_{q} column means no R^{Y} substituent is present.

### Table 4B

Table 4B is constructed the same as Table 4A, except that A is NH.

### Table 4C

Table 4C is constructed the same as Table 4A, except that A is S.

### Formulation/Utility

The compounds herein, including pharmaceutically acceptable salts can be administered as crystalline or amorphous forms, prodrugs, metabolites, hydrates, solvates, complexes, and tautomers thereof, as well as all isotopically-labelled compounds thereof. They may be administered alone or in combination with one another or with one or more pharmacologically active compounds which are different than the compounds described or specifically named herein, and the pharmaceutically acceptable salts thereof. Generally, one or more these compounds are administered as a pharmaceutical composition (a formulation) in association with one or more pharmaceutically acceptable excipients. The choice of excipients depends on the particular mode of administration, the effect of the excipient on solubility and stability, and the nature of the dosage form, among other things. Useful pharmaceutical compositions and methods for their preparation may be found, for example, in A. R. Gennaro (ed.), Remington: The Science and Practice of Pharmacy (20th ed., 2000).

Also provided herein are pharmaceutical compositions comprising a therapeutically effective amount of a compound described herein, or a pharmaceutically acceptable salt thereof, and one or more pharmaceutically acceptable carriers and/or excipients. The compounds herein, and the pharmaceutically acceptable salts thereof, can be administered orally. Oral administration may involve swallowing in which case the compound enters the bloodstream via the gastrointestinal tract. Alternatively or additionally, oral administration may involve mucosal administration (e.g., buccal, sublingual, supralingual administration) such that the compound enters the bloodstream through the oral mucosa. Formulations suitable for oral administration include solid, semi-solid and liquid systems such as tablets; soft or hard capsules containing multi- or nano-particulates, liquids, or powders; lozenges which may be liquid-filled; chews; gels; fast dispersing dosage forms; films; ovules; sprays; and buccal or mucoadhesive patches.

Liquid formulations include suspensions; solutions, syrups and elixirs. Such formulations may be employed as fillers in soft or hard capsules (made, for example, from gelatin or hydroxypropyl methylcellulose) and typically comprise a carrier (e.g., water, ethanol, polyethylene glycol, propylene glycol, methylcellulose, or a suitable oil) and one or more emulsifying agents, suspending agents or both. Liquid formulations may also be prepared by the reconstitution of a solid (e.g., from a sachet).

The compounds herein, and the pharmaceutically acceptable salts thereof, may also be used in fast-dissolving, fast-disintegrating dosage forms such as those described in Liang and Chen, Expert Opinion in Therapeutic Patents 2001, 11, 981-986.

For tablet dosage forms, depending on dose, the active pharmaceutical ingredient (API) may comprise from about 1 to about 80 wt. % of the dosage form or more typically from about 5 to about 60 wt. % of the dosage form. In addition to the API, tablets may include one or more disintegrants, binders, diluents, surfactants, glidants, lubricants, anti-oxidants, colorants, flavoring agents, preservatives, and taste-masking agents. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, C₁-C₆ alkyl-substituted hydroxypropylcellulose, starch, pregelatinized starch, and sodium alginate. Generally, the disintegrant will comprise from about 1 to about 25 wt. % or from about 5 to about 20 wt. % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropylcellulose and hydroxypropylmethylcellulose. Tablets may also contain diluents, such as lactose (monohydrate, spray-dried monohydrate, anhydrous), mannitol, xylitol, dextrose, sucrose, sorbitol, microcrystalline cellulose, starch and dibasic calcium phosphate dihydrate. Tablets may also include surface active agents, such as sodium lauryl sulfate and polysorbate, and glidants such as silicon dioxide and talc. When present, surface active agents may comprise from about 0.2 to about 5 wt. % of the tablet, and glidants may comprise from about 0.2 about 1 wt. % of the tablet. Tablets may also contain lubricants such as magnesium stearate, calcium stearate, zinc stearate, sodium stearyl fumarate, and mixtures of magnesium stearate with sodium lauryl sulfate. Lubricants may comprise from about 0.25 about 10 wt. % or from about 0.5 to about 3 wt. % of the tablet. Tablet blends may be compressed directly or by roller compaction to form tablets. Tablet blends or portions of blends may alternatively be wet-, dry-, or melt-granulated, melt-congealed, or extruded before tableting. If desired, prior to blending, one or more of the components may be sized by screening or milling or both. The final dosage form may comprise one or more layers and may be coated, uncoated, or encapsulated. Exemplary tablets may contain up to about 80 wt. % of API, from about 10 to about 90 wt. % of binder, from about 0 to about 85 wt. % of diluent, from about 2 to about 10 wt. % of disintegrant, and from about 0.25 to about 10 wt. % of lubricant. For a discussion of blending, granulation, milling, screening, tableting, coating, as well as a description of alternative techniques for preparing drug products, see A. R. Gennaro (ed.), Remington: The Science and Practice of Pharmacy (20th ed., 2000); H. A. Lieberman et al. (ed.), Pharmaceutical Dosage Forms: Tablets, Vol. 1-3 (2d ed., 1990); and D. K. Parikh &C. K. Parikh, Handbook of Pharmaceutical Granulation Technology, Vol. 81 (1997).

Consumable oral films for human or veterinary use are pliable water-soluble or water-swellable thin film dosage forms which may be rapidly dissolving or mucoadhesive. In addition to the API, a typical film includes one or more film-forming polymers, binders, solvents, humectants, plasticizers, stabilizers or emulsifiers, viscosity-modifying agents, and solvents. Other film ingredients may include anti-oxidants, colorants, flavorants and flavor enhancers, preservatives, salivary stimulating agents, cooling agents, co-solvents (including oils), emollients, bulking agents, anti-foaming agents, surfactants, and taste-masking agents. Some components of the formulation may perform more than one function. In addition to dosing requirements, the amount of API in the film may depend on its solubility. If water soluble, the API would typically comprise from about 1 to about 80 wt. % of the non-solvent components (solutes) in the film or from about 20 to about 50 wt. % of the solutes in the film. A less soluble API may comprise a greater proportion of the composition, typically up to about 88 wt. % of the non-solvent components in the film.

The film-forming polymer can be selected from natural polysaccharides, proteins, or synthetic hydrocolloids and typically comprises from about 0.01 to about 99 wt. % or from about 30 to about 80 wt. % of the film. Film dosage forms are typically prepared by evaporative drying of thin aqueous films coated onto a peelable backing support or paper, which may carried out in a drying oven or tunnel (e.g., in a combined coating-drying apparatus), in lyophilization equipment, or in a vacuum oven.

Useful solid formulations for oral administration may include immediate release formulations and modified release formulations. Modified release formulations include delayed-, sustained-, pulsed-, controlled-, targeted-, and programmed-release. For a general description of suitable modified release formulations, see U.S. Pat. No. 6,106,864. For details of other useful release technologies, such as high energy dispersions and osmotic and coated particles, see Verma et al., Pharmaceutical Technology On-line 2001 25, 1-14. Compounds herein, and the pharmaceutically acceptable salts thereof, may also be administered directly into the blood stream, muscle, or an internal organ of the subject. Suitable techniques for parenteral administration include intravenous, intraarterial, intraperitoneal, intrathecal, intraventricular, intraurethral, intrasternal, intracranial, intramuscular, intrasynovial, and subcutaneous administration. Suitable devices for parenteral administration include needle injectors, including microneedle injectors, needle-free injectors, and infusion devices.

Parenteral formulations are typically aqueous solutions which may contain excipients such as salts, carbohydrates and buffering agents (e.g., pH of from about 3 to about 9). For some applications, however, the compounds herein, and the pharmaceutically acceptable salts thereof, may be more suitably formulated as a sterile non-aqueous solution or as a dried form to be used in conjunction with a suitable vehicle such as sterile, pyrogen-free water. The preparation of parenteral formulations under sterile conditions (e.g., by lyophilization) may be readily accomplished using standard pharmaceutical techniques.

The solubility of compounds which are used in the preparation of parenteral solutions may be increased through appropriate formulation techniques, such as the incorporation of solubility-enhancing agents. Formulations for parenteral administration may be formulated to be immediate or modified release. Modified release formulations include delayed, sustained, pulsed, controlled, targeted, and programmed release. Thus, compounds herein, and the pharmaceutically acceptable salts thereof, may be formulated as a suspension, a solid, a semi-solid, or a thixotropic liquid for administration as an implanted depot providing modified release of the active compound. Examples of such formulations include drug-coated stents and semi-solids and suspensions comprising drug-loaded poly(DL-lactic-coglycolic)acid (PGLA) microspheres.

The compounds herein, and the pharmaceutically acceptable salts thereof, may also be administered topically, intradermally, or transdermally to the skin or mucosa. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers may include alcohol, water, mineral oil, liquid petrolatum, white petrolatum, glycerin, polyethylene glycol and propylene glycol. Topical formulations may also include penetration enhancers. See, e.g., Finnin and Morgan, J. Pharm. Sci. 1999, 88, 955-958. Other means of topical administration include delivery by electroporation, iontophoresis, phonophoresis, sonophoresis and microneedle or needle-free injection. Formulations for topical administration may be formulated to be immediate or modified release as described above.

The compounds herein, and the pharmaceutically acceptable salts thereof, may also be administered intranasally or by inhalation, typically in the form of a dry powder, an aerosol spray, or nasal drops. An inhaler may be used to administer the dry powder, which comprises the API alone, a powder blend of the API and a diluent, such as lactose, or a mixed component particle that includes the API and a phospholipid, such as phosphatidylcholine. For intranasal use, the powder may include a bioadhesive agent, e.g., chitosan or cyclodextrin. A pressurized container, pump, sprayer, atomizer, or nebulizer, may be used to generate the aerosol spray from a solution or suspension comprising the API, one or more agents for dispersing, solubilizing, or extending the release of the API (e.g., EtOH with or without water), one or more solvents (e.g., 1,1,1,2-tetrafluoroethane or 1,1,1,2,3,3,3-heptafluoropropane) which serve as a propellant, and an optional surfactant, such as sorbitan trioleate, oleic acid, or an oligolactic acid. An atomizer using electrohydrodynamics may be used to produce a fine mist.

Prior to use in a dry powder or suspension formulation, the drug product is usually comminuted to a particle size suitable for delivery by inhalation (typically 90% of the particles, based on volume, having a largest dimension less than 5 microns). This may be achieved by any appropriate size reduction method, such as spiral jet milling, fluid bed jet milling, supercritical fluid processing, high pressure homogenization, or spray drying.

Capsules, blisters and cartridges (made, for example, from gelatin or hydroxypropylmethyl cellulose) for use in an inhaler or insufflator maybe formulated to contain a powder mixture of the active compound, a suitable powder base such as lactose or starch, and a performance modifier such as L-Ieucine, mannitol, or magnesium stearate. The lactose may be anhydrous or monohydrated. Other suitable excipients include dextran, glucose, maltose, sorbitol, xylitol, fructose, sucrose, and trehalose.

A suitable solution formulation for use in an atomizer using electrohydrodynamics to produce a fine mist may contain from about 1 μg to about 20 mg of the API per actuation and the actuation volume may vary from about 1 μL to about 100 μL. A typical formulation may comprise one or more of the compounds herein, or a pharmaceutically acceptable salt thereof, propylene glycol, sterile water, EtOH, and NaCl. Alternative solvents, which may be used instead of propylene glycol, include glycerol and polyethylene glycol.

Formulations for inhaled administration, intranasal administration, or both, may be formulated to be immediate or modified release using, for example, PGLA. Suitable flavors, such as menthol and levomenthol, or sweeteners, such as saccharin or sodium saccharin, may be added to formulations intended for inhaled/intranasal administration.

In the case of dry powder inhalers and aerosols, the dosage unit is determined by means of a valve that delivers a metered amount. Units are typically arranged to administer a metered dose or “puff” containing from about 10 μg to about 1000 μg of the API. The overall daily dose will typically range from about 100 μg to about 10 mg which may be administered in a single dose or, more usually, as divided doses throughout the day.

The active compounds may be administered rectally or vaginally, e.g., in the form of a suppository, pessary, or enema. Cocoa butter is a traditional suppository base, but various alternatives may be used as appropriate. Formulations for rectal or vaginal administration may be formulated to be immediate or modified release as described above.

The compounds herein, and the pharmaceutically acceptable salts thereof, may also be administered directly to the eye or ear, typically in the form of drops of a micronized suspension or solution in isotonic, pH-adjusted, sterile saline. Other formulations suitable for ocular and aural administration include ointments, gels, biodegradable implants (e.g., absorbable gel sponges, collagen), non-biodegradable implants (e.g., silicone), wafers, lenses, and particulate or vesicular systems, such as niosomes or liposomes. The formulation may include one or more polymers and a preservative, such as benzalkonium chloride. Typical polymers include crossed-linked polyacrylic acid, polyvinylalcohol, hyaluronic acid, cellulosic polymers (e.g., hydroxypropylmethylcellulose, hydroxyethylcellulose, methyl cellulose), and heteropolysaccharide polymers (e.g., gelan gum). Such formulations may also be delivered by iontophoresis. Formulations for ocular or aural administration may be formulated to be immediate or modified release as described above.

As noted above, the compounds herein, and the pharmaceutically acceptable salts thereof, and their pharmaceutically active complexes, solvates and hydrates, may be combined with one another or with one or more other active pharmaceutically active compounds to treat various diseases, conditions and disorders. In such cases, the active compounds may be combined in a single dosage form as described above or may be provided in the form of a kit which is suitable for co-administration of the compositions. The kit comprises (1) two or more different pharmaceutical compositions, at least one of which contains a compound of Formula 1; and (2) a device for separately retaining the two pharmaceutical compositions, such as a divided bottle or a divided foil packet. An example of such a kit is the familiar blister pack used for the packaging of tablets or capsules. The kit is suitable for administering different types of dosage forms (e.g., oral and parenteral) or for administering different pharmaceutical compositions at separate dosing intervals, or for titrating the different pharmaceutical compositions against one another. To assist with patient compliance, the kit typically comprises directions for administration and may be provided with a memory aid.

For administration to human patients, the total daily dose of the claimed and disclosed compounds is typically in the range of about 0.1 mg to about 3000 mg depending on the route of administration. For example, oral administration may require a total daily dose of from about 1 mg to about 3000 mg, while an intravenous dose may only require a total daily dose of from about 0.1 mg to about 300 mg. The total daily dose may be administered in single or divided doses and, at the physician's discretion, may fall outside of the typical ranges given above. Although these dosages are based on an average human subject having a mass of about 60 kg to about 70 kg, the physician will be able to determine the appropriate dose for a patient (e.g., an infant) whose mass falls outside of this weight range.

The claimed and disclosed compounds may be combined with one or more other pharmacologically active compounds for the treatment of one or more related disorders, the pharmacologically active compounds can be selected from: (1) an opioid analgesic, e.g., morphine, fentanyl, codeine, etc.; (2) a nonsteroidal antiinflammatory drug (NSAID), e.g., acetaminophen, aspirin, diclofenac, etodolac, ibuprofen, naproxen, etc.; (3) a barbiturate sedative, e.g., pentobarbital; (4) a benzodiazepine having a sedative action, e.g., diazepam, lorazepam, etc.; (5) an H1 antagonist having a sedative action, e.g., diphenhydramine; (6) a sedative such as glutethimide, meprobamate, methaqualone or dichloralphenazone; (7) a skeletal muscle relaxant, e.g., baclofen, carisoprodol, chlorzoxazone, cyclobenzaprine, methocarbamol or orphrenadine; (8) an NMDA receptor antagonist; (9) an alpha-adrenergic; (10) a tricyclic antidepressant, e.g., desipramine, imipramine, amitriptyline or nortriptyline; (11) an anticonvulsant, e.g., carbamazepine, lamotrigine, topiratmate or valproate; (12) a tachykinin (NK) antagonist, particularly an NK-3, NK-2 or NK-1 antagonist; (13) a muscarinic antagonist, e.g., oxybutynin, tolterodine, etc.; (14) a COX-2 selective inhibitor, e.g., celecoxib, valdecoxib, etc.; (15) a coal-tar analgesic, in particular paracetamol; (16) a neuroleptic such as haloperidol, clozapine, olanzapine, risperidone, ziprasidone, or Miraxion®; (17) a vanilloid receptor (VR1; also known as transient receptor potential channel, TRPV1) agonist (e.g., resinferatoxin) or antagonist (e.g., capsazepine); (18) a beta-adrenergic such as propranolol; (19) a local anaesthetic such as mexiletine; (20) a corticosteroid such as dexamethasone; (21) a 5-HT receptor agonist or antagonist. particularly a 5HT_{1B/1D} agonist such as eletriptan, sumatriptan, naratriptan, zolmitriptan or rizatriptan; (22) a 5-HT_{2A} receptor antagonist such as RH-alpha-(2,3-dimethoxyphenyl)-1-[2-(4-fluorophenylethyl)]-4-piperidinemethanol (MDL-100907); (23) a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine, or a nicotine partial agonist such as varenicline; (24) Tramadol®; (25) a PDEV inhibitor; (26) an alpha-2-delta ligand such as gabapentin, pregabalin, 3-methylgabapentin, etc.; (27) a cannabinoid receptor (CB1, CB2) ligand, either agonist or antagonist such as rimonabant; (28) metabotropic glutamate subtype 1 receptor (mGluR1) antagonist; (29) a serotonin reuptake inhibitor such as sertraline, sertraline metabolite demethylsertraline, fluoxetine, etc.; (30) a noradrenaline (norepinephrine) reuptake inhibitor, such as buproprion, buproprion metabolite hydroxybuproprion, especially a selective noradrenaline reuptake inhibitor such as reboxetine, in particular (S,S)-reboxetine; (31) a dual serotonin-noradrenaline reuptake inhibitor, such as venlafaxine. O-desmethylvenlafaxine, clomipramine, desmethylclomipramine, duloxetine, milnacipran and imipramine; (32) an inducible nitricoxide synthase (iNOS) inhibitor; (33) an acetylcholinesterase inhibitor such as donepezil; (34) a prostaglandin E₂ subtype 4 (EP4) antagonist; (35) a leukotriene B4 antagonist; (36) a 5-lipoxygenase inhibitor, such as zileuton; (37) a sodium channel blocker, such as lidocaine; (38) a 5-HT3 antagonist, such as ondansetron; or (39) anti-nerve growth factor (NGF) antibodies. It is understood that the pharmaceutical agents just mentioned may be administered in the manner and at the dosages known in the art.

The compounds of this invention prepared by the methods described herein are shown in Index Table A. For mass spectral data (AP⁺(M+1)), the numerical value reported is the molecular weight of the parent molecular ion (M) formed by addition of H⁺ (molecular weight of 1) to the molecule to give a M+1 peak observed by mass spectrometry using atmospheric pressure chemical ionization (AP⁺). The alternate molecular ion peaks (e.g., M+2 or M+4) that occur with compounds containing multiple halogens are not reported.

Fragments J1 through J-17 shown below are referred to in Index Table A. The asterisk * denotes the attachment point for the fragment to the remainder of the molecule.

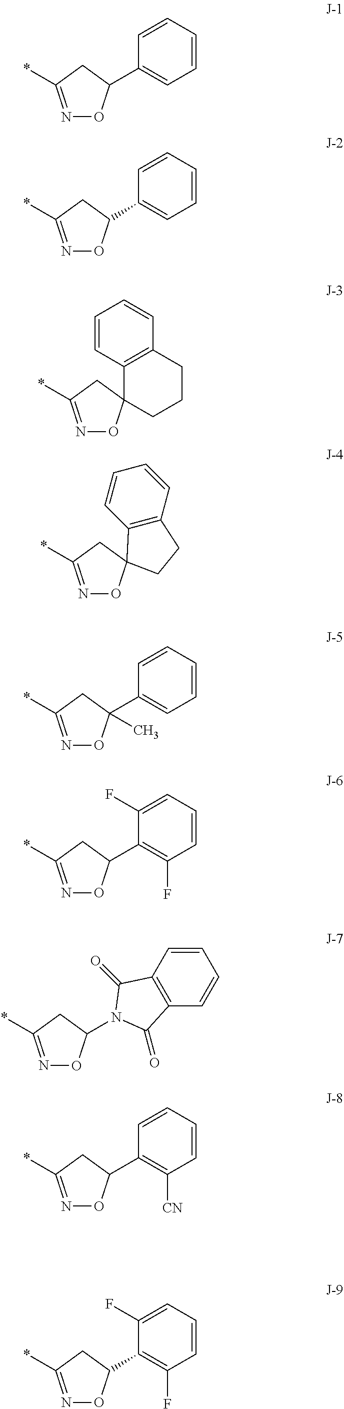

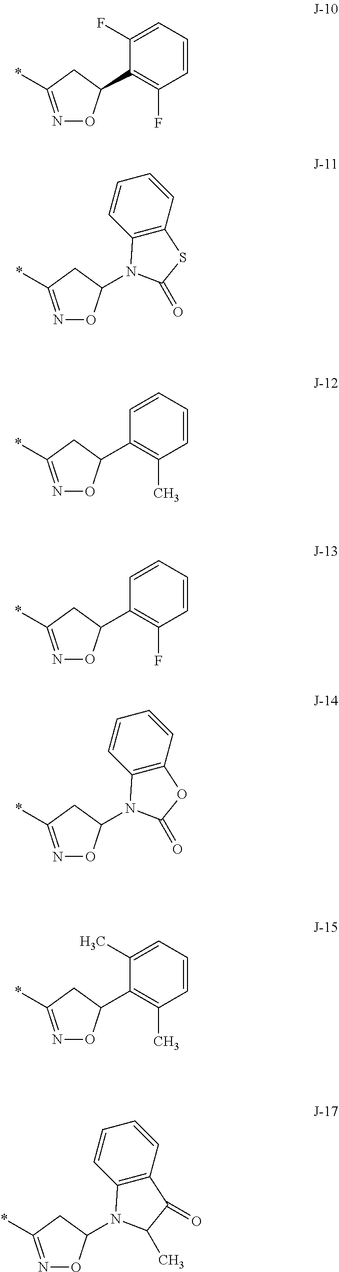

INDEX TABLE A

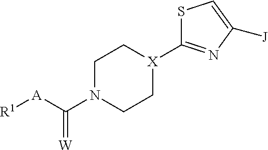

| | | | | | | AP+ |
|---|---|---|---|---|---|---|
| Cmpd. | R¹ | A | W | X | J | (M + 1) |
| 1 | phenyl | NH | O | CH | J-1 | 433 |
| 2 | 2,5-dimethylphenyl | NH | O | CH | J-1 | 461 |
| 3 | 2,5-dichlorophenyl | NH | O | CH | J-1 | 500 |
| 4 | 6-bromopyridin-2-yl | NH | O | CH | J-1 | 511 |
| 5 | 6-trifluoromethylpyridin-2-yl | NH | O | CH | J-1 | 502 |
| 6 | 4-trifluoromethylpyridin-2-yl | NH | O | CH | J-1 | 502 |
| 7 | 3-cyanophenyl | NH | O | CH | J-1 | 458 |
| 8 | 2-methoxy-5-chlorophenyl | NH | O | CH | J-1 | 497 |
| 9 | 3-chloropyridin-2-yl | NH | O | CH | J-1 | 468 |
| 10 | 2,5-dimethoxyphenyl | NH | O | CH | J-1 | 493 |
| 11 | phenyl | O | O | CH | J-1 | 434 |
| 12 | 2-methylphenyl | NH | O | CH | J-1 | 447 |
| 13 | 2-trifluoromethylphenyl | NH | O | CH | J-1 | 501 |
| 14 | 3-trifluoromethylphenyl | NH | O | CH | J-1 | 501 |
| 15 | 2,5-difluorophenyl | NH | O | CH | J-1 | 469 |
| 16 | 2-methyl-5-chlorophenyl | NH | O | CH | J-1 | 481 |
| 17 | 2,5-dimethylphenyl | NH | O | CH | J-2 | 461 |
| 18 | 1,3-dimethylpyrazol-5-y1 | NH | O | CH | J-1 | 451 |
| 19 | 2,5-dimethylphenyl | NH | O | CH | J-3 | 501 |
| 20 | 2,5-dimethylphenyl | NH | O | CH | J-4 | 487 |
| 21 | 2,5-dimethylphenyl | NH | O | CH | J-5 | 475 |
| 22 | 1-methyl-3- | NH | O | CH | J-1 | 505 |
| | trifluoromethylpyrazol-5-yl | | | | | |
| 23 | 2,5-dimethylphenyl | O | O | CH | J-1 | 462 |
| 24 | 3-trifluoromethyl-5- | NH | O | CH | J-1 | 505 |
| | methylpyrazol-1-yl | | | | | |
| 25 | 4-trifluoromethylthiazol-2-yl | NH | O | CH | J-1 | 508 |
| 26 | 2-methyl-5-bromophenyl | NH | O | CH | J-1 | 525 |
| 27 | 1-ethylpyrazol-5-yl | NH | O | CH | J-1 | 451 |
| 28 | 3,5-dimethylphenyl | NH | O | CH | J-1 | 461 |
| 29 | 2-chloropyridin-3-yl | NH | O | CH | J-1 | 468 |
| 30 | 4,6-dimethylpyrimidin-2-yl | NH | O | CH | J-1 | 463 |
| 31 | 2-chloro-5-cyanophenyl | NH | O | CH | J-1 | 492 |
| 32 | 2-chloro-5-methylphenyl | NH | O | CH | J-1 | 480 |
| 33 | pyridin-3-yl | NH | O | CH | J-1 | 434 |
| 34 | 2,5-dichloropyridin-3-yl | NH | O | CH | J-1 | 502 |
| 35 | 2-methyl-5-trifluoromethylphenyl | NH | O | CH | J-1 | 515 |
| 36 | 2,5-dibromophenyl | NH | O | CH | J-1 | 589 |
| 37 | 2-methyl-5-isopropylphenyl | NH | O | CH | J-1 | 489 |
| 38 | 2-chloro-5-trifluoromethylphenyl | NH | O | CH | J-1 | 535 |
| 39 | 2-methoxy-5-trifluoromethylphenyl | NH | O | CH | J-1 | 531 |
| 40 | 3-trifluoromethoxyphenyl | NH | O | CH | J-1 | 517 |
| 41 | 3,4-dimethylisoxazol-5-yl | NH | O | CH | J-1 | 452 |
| 42 | 3-methylisoxazol-5-yl | NH | O | CH | J-1 | 438 |
| 43 | 2-chloro-5-methylpyridin-3-yl | NH | O | CH | J-1 | 481 |
| 44 | 2,6-bistrifluoromethylpyridin-4-yl | NH | O | CH | J-1 | 570 |
| 45 | 4-nitrophenyl | O | O | CH | J-1 | 479 |
| 46 | 2,5-dimethylphenyl | NH | O | CH | J-6 | 497 |
| 47 | 3,5-dimethylpyrazol-1-yl | NH | O | CH | J-1 | 451 |
| 48 | benzothiazol-2-yl | NH | O | CH | J-1 | 490 |
| 49 | thiazol-2-yl | NH | O | CH | J-1 | 440 |
| 50 | 2,5-dimethylphenyl | NH | O | CH | J-7 | 530 |
| 51 | 2,5-dimethylphenyl | NH | O | CH | J-8 | 486 |
| 52 | phenyl | O | O | CH | J-3 | 474 |
| 53 | 2-chlorophenyl | NH | O | CH | J-1 | 467 |
| 54 | 2-methoxyphenyl | NH | O | CH | J-1 | 463 |
| 55 | 2-ethyl-1,3,4-thiadiazol-5-yl | NH | O | CH | J-1 | 469 |
| 56 | 2-methyl-1,3,4-thiadiazol-5-yl | NH | O | CH | J-1 | 455 |
| 57 | 3-methylphenyl | O | O | CH | J-1 | 448 |
| 58 | 4-methylphenyl | O | O | CH | J-1 | 448 |
| 59 | 2,5-dimethylphenyl [note 1] | NH | O | CH | J-9 | 497 |
| 60 | 2,5-dimethylphenyl [note 2] | NH | O | CH | J-10 | 497 |
| 61 | 2-chlorophenyl | O | O | CH | J-1 | 468 |
| 62 | 2-methylphenyl | O | O | CH | J-1 | 448 |
| 63 | 3,5-dimethylphenyl | O | O | CH | J-1 | 462 |
| 64 | 2,6-dimethylphenyl | O | O | CH | J-1 | 462 |
| 65 | 3-chlorophenyl | O | O | CH | J-1 | 468 |
| 66 | 4-chlorophenyl | O | O | CH | J-1 | 468 |
| 67 | 2,5-dimethylphenyl | NH | O | CH | J-11 | 534 |
| 68 | 2,5-dimethylphenyl | NH | O | CH | J-12 | 475 |
| 69 | 2,5-dimethylphenyl | NH | O | CH | J-13 | 479 |
| 70 | 2,5-dimethylphenyl | NH | O | N | J-1 | 462 |
| 71 | 2,5-dimethylphenyl | NH | O | CH | J-14 | 518 |
| 72 | 2,5-dimethylphenyl | NH | O | CH | J-15 | 489 |
| 74 | 2,5-dimethylphenyl | NH | O | CH | J-17 | 516 |
| 75 | 2,5-dimethylphenyl | NH | S | CH | J-1 | 477 |
| 76 | 2,5-dimethylphenyl | NH | S | CH | J-6 | 513 |

Note 1:
Faster eluting enantiomer from the CHIRACEL ® OJ-RH column using 1:1 acetonitrile:methanol in water as eluant. Analysis using analytical CHIRACEL ® OJ-RH column indicated about 99% optical purity.
Note 2:
Slower eluting enantiomer from the CHIRACEL ® OJ-RH column using 1:1 acetonitrile:methanol in water as eluant. Analysis using analytical CHIRACEL ® OJ-RH column indicated about 100% optical purity.

### BIOLOGICAL EXAMPLES OF THE INVENTION

The compounds of this invention listed in Index Table A were tested according to the following protocols.

### In Vitro Evaluation of FAAH Inhibition

FAAH Expression and Purification—recombinant human FAAH was expressed in truncated form, in which the transmembrane (TM) portion of the enzyme was removed from the N-terminal (amino acids 1-33), and then heterologously expressed as a MBP (maltose-binding protein) fusion protein in E. coli (MBP-ΔTM-FAAH) similar to the procedure described by Labar, G. et al. Amino acids 2008, 34, 127-133. The region of the gene corresponding to amino acids 34 to 579 was cloned into pMAL-c4x (New England BioLabs, Inc.) using EcoR1 and SalI restriction sites. E. coli T7 Express cells, containing the FAAH constructs, were used for expression of protein by induction with IPTG (isopropyl-β-D-thiogalactopyranoside) (100 μM) overnight at room temperature in Lennox Broth with 0.2% glucose. After harvest, the cells were resuspended in 20 mM Hepes buffer (pH 7.4) containing 200 mM NaCl, 2 mM DTT (dithiothreitol). The cell suspension was lysed by sonication, and the cell debris removed by centrifugation. The soluble extract was adjusted to 2.5 mg/mL protein, and the FAAH fusion protein (˜105 kDa) loaded onto a 5 mL column of amylose affinity resin. The enzyme was eluted using 15 mM maltose as per manufacturer's (New England BioLabs, Inc.) instruction. Fractions containing FAAH were concentrated and further purified using Sephacryl™ 5100 (HIPrep™ 26/60, GE Healthcare, Inc.) chromatography. Fractions enriched in FAAH were pooled, concentrated, and made 10% in glycerol then stored at −80° C. until use. All column chromatography steps used the Hepes buffer described above.

FAAH assay—Enzyme activity was measured using the fluorogenic substrate, decanoyl 7-amino-4-methylcoumarin (D-AMC) as described by Kage, K. L. et al. J. of Neuroscience Methods 2007, 161, 47-54. Briefly, the assay buffer consisted of 125 mM Tris-CL, 1 mM EDTA, and 0.1% BSA (pH 8.0). D-AMC was used at final concentration of 5 μM in all assays. Reactions were carried out in black 96-well microplates (Costar, Inc) using a SpectraMax Gemini™ (Molecular Devices, Inc.) fluorescence plate reader in a reaction volume of 200 μL per well at 37° C. Reaction rates were monitored at an emission wavelength of 430 nm using an excitation wavelength of 351 nm over 30 to 40 minutes. Experimental compounds were initially evaluated at a single concentration of 2 μM. Compounds inhibiting the reaction >90% were subsequently retested to determine IC₅₀ values. Representative results for compounds tested in the assay are listed in Table A.

TABLE A

| Compound | IC₅₀ (nM) |
|---|---|
| 1 | 190 |
| 11 | 0.04 |
| 12 | 600 |
| 23 | 35 |
| 49 | 220 |
| 52 | 29 |
| 57 | 0.6 |
| 58 | 0.2 |
| 61 | 0.06 |
| 62 | 0.04 |
| 63 | 42 |
| 64 | 2000 |
| 65 | 0.41 |
| 66 | 0.08 |

### Evaluation of FAAH Inhibitor Selectivity

The specificity of FAAH inhibition relative to other mechanistically similar enzymes, such as porcine liver esterase and porcine pancreatic elastase, was also explored for selected compounds. Both enzymes and substrates were obtained from commercial sources, and assayed in microplate format. N-succinyl-ala-ala-ala-p-nitroanilide was used as a substrate for pancreatic elastase, and 4-nitrophenyl butyrate was used as a substrate for measuring liver esterase activity. Briefly, enzyme activity was measured by following the release of p-nitroaniline and p-nitrophenol at 400 nm from the respective chromogenic substrates using a SpectraMax™ Plus (Molecular Devices, Inc.) plate reader. The assay reaction mixture contained enzyme, 100 uM substrate, 0.125 M TrisCl, and 0.2 mM EDTA, pH 8.0 in a total volume of 200 μL. Reactions were started by the addition of substrate. Control reactions give linear reaction rates (20 to 50 mOD/min) over at least 5 min Table B describes IC₅₀ results for a series of selected compounds. All compounds tested showed at most, only slight inhibition of pancreatic elastase at the highest concentration tested (10 μM). Several compounds should some level of inhibition of liver esterase, but IC₅₀ values were orders of magnitude less potent compared to FAAH inhibition. These results indicated a high degree of specificity for FAAH inhibition by these compounds.

TABLE B

| | IC₅₀ (nM) Porcine | IC₅₀ (nM) Porcine |
|---|---|---|
| Compound | Liver Esterase | Elastase Pancreas |
| 49 | >10 | >10 |
| 1 | 5.6 | >10 |
| 61 | 0.87 | >10 |
| 66 | 0.47 | >10 |
| 62 | 0.84 | >10 |
| 11 | 2.8 | >10 |

Inhibition of porcine esterase and elastase were measured using 4-nitophenyl butyrate and N-succinyl-ala-ala-ala-p-nitroanilide as substrates respectively.

### Evaluation of Analgesic Potential of FAAH Inhibitors by Tail Immersion Assy in Mice

The analgesic potential of Compounds 1, 11, 61 and 49 were determined by tail immersion assay. Anandamide (a brain lipid involved in natural analgesic response) was used as negative control, and OL-135 alone (an inhibitor of fatty acid amide hydrolase that metabolizes anandamide) and a combination of OL-135 and anandamide were used as positive controls. Two vehicle controls (2:2:16 DMSO:Alkamuls:saline and 1:1:18 EtOH:Alkamuls:saline) were also evaluated. Previous research indicates that administration of anandamide alone is largely ineffective in causing hypothermia or analgesia. However, when anandamide is administered along with OL-135, the analgesic effect was significantly elevated (A. H. Lichtman, et al. The Journal of Pharmacology and Experimental Therapeutics 2004, 311, 441-448) Since Compounds 1, 11, 61 and 49 were shown to inhibit FAAH in vitro, the potential analgesic effects of these compounds were assessed by administering them in combination with anandamide in the present screening study. Test substances were injected once by intraperitoneal (i.p.) route to female Cr1:CD1(ICR) mice. The tail immersion assay was conducted prior to administration of compounds to establish baseline values and again after administration of compounds.

Analgesia was evaluated in female mice by immersing approximately 3.5 cm of each tail into water that was maintained at 52+/−1° C. for a maximum of 10 seconds (sec). The length of time until the animal removed its tail from the water or made a significant tail movement was measured. If the response time was less than 5 sec, a second trial was conducted. The test data are shown in Table C.

A preliminary study was conducted to determine the optimal time interval between administration of OL-135 or the test substances and the administration of anandamide, and to determine the time interval between treatment with anandamide and conducting the tail immersion assay. Based on the results of the preliminary study, the time interval between administration of the test substances and anandamide was established as 40 minutes. In addition, the time interval between administration of anandamide and conducting the tail immersion assay was established to be 40 minutes.

The formulations were made on the day of dosing and administered once by intraperitoneal route. Anandamide, OL-135, Compound 61 and Compound 49 were formulated in Vehicle 2 and Compound 1 and Compound 11 were formulated in Vehicle 1

Because the maximum mean response time of two vehicle controls, a negative control, and baseline evaluations of all groups was 7.5 sec, the treatments showing a mean response time equal to or below 7.5 sec were considered as having no analgesic effect. The mean response times with Compounds 1 and 49 were lower than 7.5 sec, and therefore, these compounds were considered to show no analgesic effects at the rate tested. Compounds 11 and 61 provided mean response times of >7.5 sec, and 100% and 90% of the treated animals, respectively, exhibited the maximum measured response time of 10 sec. Therefore, Compounds 11 and 61 were considered to show analgesic effects at the rate tested.

### Study Design

| | Number | | |
|---|---|---|---|
| Compound | of Mice | Dose 1 (mg/kg) | Dose 2 (mg/kg) |
| Vehicle 1 | 5 | 2:2:16 | — |
| | | DMSO:alkamuls:saline | |
| Vehicle 2 | 5 | 1:1:18 | — |
| | | EtOH:alkamuls:saline | |
| Positive Control | 10 | OL-135 (10) | Anandamide (50) |
| Negative Control | 10 | — | Anandamide (50) |
| Compound 1 | 10 | Compound 1 (10) | Anandamide (50) |
| Compound 11 | 10 | Compound 11 (10) | Anandamide (50) |
| Compound 61 | 10 | Compound 61 (10) | Anandamide (50) |
| Compound 49 | 10 | Compound 49 (10) | Anandamide (50) |

TABLE C

| | Baseline Time | | | | | |
|---|---|---|---|---|---|---|
| Compound | (sec)Test | | Time (sec) | | % MPE | |
| Vehicle 1 | 4.0 | (1.6) | 4.6 | (1.6) | −1.0% | (56.8%) |
| Vehicle 2 | 7.0 | (2.4) | 5.2 | (1.7) | −23.9% | (31.1%) |
| Positive Control | 4.1 | (1.9) | 7.6 | (2.4) | 61.5% | (41.5%) |
| Negative Control | 6.9 | (3.1) | 6.7 | (3.0) | 20.3% | (44.0%) |
| Compound 1 | 3.5 | (0.9) | 5.0 | (1.1) | 20.2% | (27.5%) |
| Compound 11 | 5.8 | (2.7) | 10.0 | (0.0) | 100.0% | (0.0%) |
| Compound 61 | 6.0 | (3.0) | 9.4 | (1.9) | 86.9% | (34.6%) |
| Compound 49 | 7.5 | (2.9) | 6.4 | (2.9) | −4.0% | (71.8%) |

Data presented as Mean (Standard Deviation)
% MPE is percent of the maximum possible effect (test-baseline)/(10-baseline)

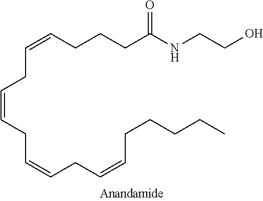 [SMILES: CCCCC/C=C\C/C=C\C/C=C\C/C=C\CCCC(=O)NCCO]

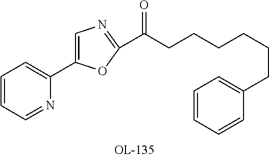 [SMILES: O=C(CCCCCCc1ccccc1)c1ncc(-c2ccccn2)o1]

What is claimed is:

1. A compound selected from the compounds of Formula 1, N-oxides and salts thereof, 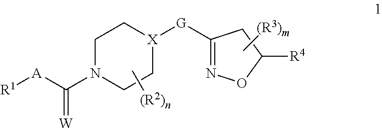
wherein
A is O or S;
W is O or S;
X is CR^{2a} or N;
R¹ is phenyl, naphthalenyl or 1,2-benzisoxazol-3-yl, each optionally substituted with up to 3 substituents independently selected from R^{5a}; or a 5- to 6-membered heteroaromatic ring, the ring containing ring members selected from carbon atoms and 1 to 4 heteroatoms independently selected from up to 2 O, up to 2 S and up to 4 N atoms, the ring optionally substituted with up to 3 substituents independently selected from R^{5a} on carbon atom ring members and R^{5b} on nitrogen atom ring members;
each R² is independently halogen, cyano, hydroxy, C₁-C₂ alkyl, C₁-C₂ haloalkyl or C₁-C₂ alkoxy;
R^{2a} is H, halogen, cyano, hydroxy, C₁-C₂ alkyl, C₁-C₂ haloalkyl or C₁-C₂ alkoxy;
each R³ is independently halogen, cyano, C₁-C₃ alkyl or C₁-C₃ haloalkyl;
R⁴ is C₁-C₈ alkyl, C₁-C₈ haloalkyl, C₃-C₈ cycloalkyl, C₃-C₈ halocycloalkyl, C₄-C₁₀ alkylcycloalkyl, C₄-C₁₀ cycloalkylalkyl, C₂-C₈ alkoxyalkyl, C₂-C₈ haloalkoxyalkyl, C₄-C₁₀ cycloalkoxyalkyl, C₃-C₈ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₂-C₆ haloalkylaminoalkyl, C₃-C₈ dialkylaminoalkyl, C₄-C₁₀ cycloalkylaminoalkyl, C₁-C₆ hydroxyalkyl, C₂-C₆ alkylcarbonyl, C₂-C₆ haloalkylcarbonyl, C₂-C₆ alkoxycarbonyl, C₂-C₆ alkylaminocarbonyl or C₃-C₈ dialkylaminocarbonyl; or benzyl, phenyl, naphthalenyl, 1,3-dihydro-1,3-dioxo-2H-isoindol-2-yl, 2-oxo-3(2H)-benzooxazol-3-yl or 2-oxo-3(2H)-benzothiazol-3-yl or each optionally substituted with up to 3 substituents independently selected from R^{8a}; or a 5- to 6-membered heteroaromatic ring, the ring optionally substituted with up to 3 substituents independently selected from R^{8a} on carbon atom ring members and R^{8b} on nitrogen atom ring members;
each R^{5a} is independently halogen, hydroxy, amino, cyano, nitro, C₁-C₄ alkyl, C₁-C₆ haloalkyl, C₃-C₆ cycloalkyl, C₃-C₆ halocycloalkyl, C₂-C₄ alkoxyalkyl, C₁-C₄ hydroxyalkyl, C₁-C₄ alkoxy, C₁-C₄ haloalkoxy, C₁-C₄ alkylthio, C₁-C₄ haloalkylthio, C₁-C₄ alkylsulfinyl, C₁-C₄ alkylsulfonyl, C₁-C₄ haloalkylsulfinyl, C₁-C₄ haloalkylsulfonyl, C₁-C₄ alkylamino, C₂-C₈ dialkylamino, C₂-C₄ alkylcarbonyl, C₂-C₆ alkoxycarbonyl, C₂-C₆ alkylaminocarbonyl, C₃-C₈ dialkylaminocarbonyl, C₂-C₆ alkylcarbonyloxy, C₂-C₆ alkylcarbonylthio or C₃-C₆ trialkylsilyl;
each R^{5b} is independently C₁-C₄ alkyl, C₃-C₄ alkenyl, C₃-C₄ alkynyl, C₃-C₆ cycloalkyl, C₁-C₄ haloalkyl, C₃-C₄ haloalkenyl, C₃-C₄ haloalkynyl, C₃-C₆ halocycloalkyl or C₂-C₄ alkoxyalkyl;
G is a 5-membered heteroaromatic ring, the ring containing ring members selected from carbon atoms and 1 to 3 heteroatoms independently selected from up to 2 O, up to 2 S and up to 3 N atoms, the ring optionally substituted with up to 1 substituent selected from R^{7a} on a carbon atom and R^{7b} on a nitrogen atom;
R^{7a} is halogen, cyano, C₁-C₂ alkyl or C₁-C₂ haloalkyl;
R^{7b} is C₁-C₂ alkyl or C₁-C₂ haloalkyl;
each R^{8a} is independently halogen, hydroxy, amino, cyano, nitro, C₁-C₄ alkyl, C₁-C₄ haloalkyl, C₁-C₄ alkoxy, C₁-C₄ haloalkoxy, C₁-C₄ alkylthio, C₁-C₄ haloalkylthio, C₁-C₄ alkylsulfinyl, C₁-C₄ alkylsulfonyl, C₁-C₄ haloalkylsulfinyl, C₁-C₄ haloalkylsulfonyl, C₁-C₄ alkylamino, C₂-C₆ dialkylamino, C₂-C₄ alkylcarbonyl, C₂-C₆ alkoxycarbonyl, C₂-C₆ alkylaminocarbonyl or C₃-C₈ dialkylaminocarbonyl; or
a pair of R^{8a} and R³ are taken together with the atoms to which they are attached to form a 5- to 7-membered ring, the ring containing ring members selected from carbon atoms and up to 2 heteroatoms independently selected from up to 1 O, up to 1 S and up to 1 N, wherein up to 2 carbon atom ring members are independently selected from C(═O) and C(═S), and the sulfur atom ring members are independently selected from S(═O)ᵤ(═NR¹⁰)_{z}, the ring optionally substituted with up to 2 substituents independently selected from R^{9a} on carbon atom ring members and from R^{9b} on a nitrogen atom ring member;
each R^{8b} is independently C₁-C₄ alkyl or C₁-C₄ haloalkyl; or
a pair of R^{8b} and R³ are taken together with the atoms to which they are attached to form a 5- to 7-membered ring, the ring containing ring members selected from carbon atoms and up to 2 heteroatoms independently selected from up to 1 O, up to 1 S and up to 1 N, wherein up to 2 carbon atom ring members are independently selected from C(═O) and C(═S), and the sulfur atom ring members are independently selected from S(═O)ᵤ(═NR¹⁰)_{z}, the ring optionally substituted with up to 2 substituents independently selected from R^{9a} on carbon atom ring members and from R^{9b} on a nitrogen atom ring member;
each R^{9a} is independently halogen, C₁-C₄ alkyl, C₁-C₄ haloalkyl, C₁-C₄ alkoxy, C₁-C₄ haloalkoxy, C₁-C₄ alkylthio or C₁-C₄ haloalkylthio;
R^{9b} is C₁-C₄ alkyl or C₁-C₄ haloalkyl;
R¹⁰ is independently H, C₁-C₄ alkyl, C₂-C₄ alkenyl, C₂-C₄ alkynyl, C₁-C₄ haloalkyl, C₂-C₄ haloalkenyl, C₂-C₄ haloalkynyl, C₂-C₄ alkoxyalkyl, C₂-C₄ alkylcarbonyl, C₂-C₄ haloalkylcarbonyl, C₁-C₄ alkylsulfonyl or C₁-C₄ haloalkylsulfonyl;
m is 0, 1 or 2;
n is 0, 1 or 2; and
u and z in the instance of)S(═O)ᵤ(═NR¹⁰)_{z} are independently 0, 1 or 2, provided that the sum of u and z in the instance of S(═O)ᵤ(═NR¹⁰)_{z} is 0, 1 or 2;
provided that when X is N, then G is attached to X through a carbon atom ring member.

2. A compound of claim 1 wherein
R¹ is selected from U-1 through U-51 as shown in Exhibit 1 wherein each R^{V} is independently selected from H and R^{5a} when R^{V} is attached to a carbon atom ring member, and R^{V} is selected from H and R^{5b} when R^{V} is attached to a nitrogen atom ring member, and the bond projecting to the left is bonded to A of Formula 1;
k is 0, 1, 2 or 3;
R⁴ is benzyl, phenyl or naphthalenyl, each optionally substituted with up to 3 substituents independently selected from R^{8a}; or pyridinyl, thienyl, pyrazolyl, triazolyl or imidazolyl, each optionally substituted with up to 3 substituents independently selected from R^{8a} on carbon atom ring members and R^{8b} on a nitrogen atom ring member;
G is selected from G-1 through G-48 as shown in Exhibit 2 wherein R^{Y} is selected from H and R^{7a} when R^{Y} is attached to a carbon atom ring member, and R^{Y} is selected from H and R^{7b} when R^{Y} is attached to a nitrogen atom ring member, and the bond projecting to the left is bonded to X and the bond projecting to the right is bonded to the isoxazole ring in Formula 1; and
q is 0 or 1.

3. A compound of claim 2 wherein
A is O;
W is O;
X is CR^{2a};
R¹ is selected from U-21 and U-37 through U-51;
each R² is independently C₁-C₂ alkyl or C₁-C₂ haloalkyl;
R^{2a} is H;
each R³ is independently cyano or C₁-C₃ alkyl;
R⁴ is benzyl or phenyl, each optionally substituted with up to 3 substituents independently selected from R^{8a}; or pyridinyl or thienyl, each optionally substituted with up to 3 substituents independently selected from R^{8a} on carbon atom ring members;
each R^{5a} is independently halogen, hydroxy, cyano, nitro, C₁-C₄ alkyl, C₁-C₆ haloalkyl, C₁-C₄ alkoxy, C₁-C₄ haloalkoxy, C₁-C₄ alkylthio, C₁-C₄ haloalkylthio, C₁-C₄ alkylsulfonyl, C₁-C₄ alkylsulfonyl, C₁-C₄ haloalkylsulfinyl, C₁-C₄ haloalkylsulfonyl, C₂-C₈ dialkylamino, C₂-C₄ alkylcarbonyl, C₂-C₆ alkoxycarbonyl or C₂-C₆ alkylcarbonyloxy;
G is selected from G-25 through G-34 and G-43 through G-48;
each R^{8a} is independently halogen, hydroxy, amino, cyano, nitro, C₁-C₃ alkyl, C₁-C₃ haloalkyl, C₁-C₃ alkoxy, C₁-C₃ haloalkoxy, C₁-C₃ alkylthio or C₁-C₃ haloalkylthio;
n is 0 or 1; and
q is 0.

4. A compound of claim 3 wherein
R¹ is selected from U-21, U-37, U-38, U-39, U-42, U-44, U-50 and U-51;
each R^{5a} is independently halogen, cyano, nitro, C₁-C₂ alkyl, C₁-C₂ haloalkyl, C₁-C₂ alkoxy or C₁-C₂ haloalkoxy;
R⁴ is a phenyl ring optionally substituted with up to 3 substituents independently selected from R^{8a};
n is 0; and
m is 0 or 1.

5. A compound of claim 4 wherein
R¹ is selected from U-21, U-50 and U-51;
R³ is cyano or C₁-C₂ alkyl;
each R^{5a} is independently halogen, nitro, C₁-C₂ alkyl, C₁-C₂ haloalkyl or C₁-C₂ alkoxy; and
G is selected from G-26, G-34, G-43 and G-47.

6. A compound of claim 4 wherein
R¹ is U-50;
R⁴ is a phenyl;
each R^{5a} is independently bromo, chloro, methyl, trifluoromethyl or methoxy;
G is G-26; and
m is 0.

7. A compound of claim 1 selected from the group consisting of:
phenyl 4-[4-(4,5-dihydro-5-phenyl-3-isoxazolyl)-2-thiazolyl]-1-piperidinecarboxylate and
2-chlorophenyl 4-[4-(4,5-dihydro-5-phenyl-3-isoxazolyl)-2-thiazolyl]-1-piperidine-carboxylate.

8. A method for inhibiting fatty acid amide hydrolase activity in a subject, said method comprising administering to the subject a compound of Formula 1, an N-oxide or pharmaceutically acceptable salt thereof, to achieve a serum concentration sufficient to inhibit fatty acid amide hydrolase activity in the subject, wherein
A is O, S or NR⁶;
W is O or S;
X is CR^{2a} or N;
R¹ is phenyl, naphthalenyl or 1,2-benzisoxazol-3-yl, each optionally substituted with up to 3 substituents independently selected from R^{5a}; or a 5- to 6-membered heteroaromatic ring, the ring containing ring members selected from carbon atoms and 1 to 4 heteroatoms independently selected from up to 2 O, up to 2 S and up to 4 N atoms, the ring optionally substituted with up to 3 substituents independently selected from R^{5a} on carbon atom ring members and R^{5b} on nitrogen atom ring members;
each R² is independently halogen, cyano, hydroxy, C₁-C₂ alkyl, C₁-C₂ haloalkyl or C₁-C₂ alkoxy;
R^{2a} is H, halogen, cyano, hydroxy, C₁-C₂ alkyl, C₁-C₂ haloalkyl or C₁-C₂ alkoxy;
each R³ is independently halogen, cyano, C₁-C₃ alkyl or C₁-C₃ haloalkyl;
R⁴ is C₁-C₈ alkyl, C₁-C₈ haloalkyl, C₃-C₈ cycloalkyl, C₃-C₈ halocycloalkyl, C₄-C₁₀ alkylcycloalkyl, C₄-C₁₀ cycloalkylalkyl, C₂-C₈ alkoxyalkyl, C₂-C₈ haloalkoxyalkyl, C₄-C₁₀ cycloalkoxyalkyl, C₃-C₈ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₂-C₆ haloalkylaminoalkyl, C₃-C₈ dialkylaminoalkyl, C₄-C₁₀ cycloalkylaminoalkyl, C₁-C₆ hydroxyalkyl, C₂-C₆ alkylcarbonyl, C₂-C₆ haloalkylcarbonyl, C₂-C₆ alkoxycarbonyl, C₂-C₆ alkylaminocarbonyl or C₃-C₈ dialkylaminocarbonyl; or benzyl, phenyl, naphthalenyl, 1,3-dihydro-1,3-dioxo-2H-isoindol-2-yl, 2-oxo-3(2H)-benzooxazol-3-yl or 2-oxo-3(2H)-benzothiazol-3-yl or each optionally substituted with up to 3 substituents independently selected from R^{8a}; or a 5- to 6-membered heteroaromatic ring, the ring optionally substituted with up to 3 substituents independently selected from R^{8a} on carbon atom ring members and R^{8b} on nitrogen atom ring members;
each R^{5a} is independently halogen, hydroxy, amino, cyano, nitro, C₁-C₄ alkyl, C₁-C₆ haloalkyl, C₃-C₆ cycloalkyl, C₃-C₆ halocycloalkyl, C₂-C₄ alkoxyalkyl, C₁-C₄ hydroxyalkyl, C₁-C₄ alkoxy, C₁-C₄ haloalkoxy, C₁-C₄ alkylthio, C₁-C₄ haloalkylthio, C₁-C₄ alkylsulfinyl, C₁-C₄ alkylsulfonyl, C₁-C₄ haloalkylsulfinyl, C₁-C₄ haloalkylsulfonyl, C₁-C₄ alkylamino, C₂-C₈ dialkylamino, C₂-C₄ alkylcarbonyl, C₂-C₆ alkoxycarbonyl, C₂-C₆ alkylaminocarbonyl, C₃-C₈ dialkylaminocarbonyl, C₂-C₆ alkylcarbonyloxy, C₂-C₆ alkylcarbonylthio or C₃-C₆ trialkylsilyl;
each R^{5b} is independently C₁-C₄ alkyl, C₃-C₄ alkenyl, C₃-C₄ alkynyl, C₃-C₆ cycloalkyl, C₁-C₄ haloalkyl, C₃-C₄ haloalkenyl, C₃-C₄ haloalkynyl, C₃-C₆ halocycloalkyl or C₂-C₄ alkoxyalkyl;
R⁶ is H, C₁-C₄ alkyl, C₂-C₄ alkenyl, C₂-C₄ alkynyl, C₁-C₄ haloalkyl, C₂-C₄ haloalkenyl, C₂-C₄ haloalkynyl, C₂-C₄ alkoxyalkyl, C₂-C₄ alkylcarbonyl, C₂-C₄ haloalkylcarbonyl, C₁-C₄ alkylsulfonyl or C₁-C₄ haloalkylsulfonyl;
G is a 5-membered heteroaromatic ring, the ring containing ring members selected from carbon atoms and 1 to 3 heteroatoms independently selected from up to 2 O, up to 2 S and up to 3 N atoms, the ring optionally substituted with up to 1 substituent selected from R^{7a} on a carbon atom and R^{7b} on a nitrogen atom;
R^{7a} is halogen, cyano, C₁-C₂ alkyl or C₁-C₂ haloalkyl;
R^{7b} is C₁-C₂ alkyl or C₁-C₂ haloalkyl;
each R^{8a} is independently halogen, hydroxy, amino, cyano, nitro, C₁-C₄ alkyl, C₁-C₄ haloalkyl, C₁-C₄ alkoxy, C₁-C₄ haloalkoxy, C₁-C₄ alkylthio, C₁-C₄ haloalkylthio, C₁-C₄ alkylsulfinyl, C₁-C₄ alkylsulfonyl, C₁-C₄ haloalkylsulfinyl, C₁-C₄ haloalkylsulfonyl, C₁-C₄ alkylamino, C₂-C₆ dialkylamino, C₂-C₄ alkylcarbonyl, C₂-C₆ alkoxycarbonyl, C₂-C₆ alkylaminocarbonyl or C₃-C₈ dialkylaminocarbonyl; or
a pair of R^{8a} and R³ are taken together with the atoms to which they are attached to form a 5- to 7-membered ring, the ring containing ring members selected from carbon atoms and up to 2 heteroatoms independently selected from up to 1 O, up to 1 S and up to 1 N, wherein up to 2 carbon atom ring members are independently selected from C(═O) and C(═S), and the sulfur atom ring members are independently selected from)S(═O)ᵤ(═NR¹⁰)_{z}, the ring optionally substituted with up to 2 substituents independently selected from R^{9a} on carbon atom ring members and from R^{9b} on a nitrogen atom ring member;
each R^{8b} is independently C₁-C₄ alkyl or C₁-C₄ haloalkyl; or
a pair of R^{8b} and R³ are taken together with the atoms to which they are attached to form a 5- to 7-membered ring, the ring containing ring members selected from carbon atoms and up to 2 heteroatoms independently selected from up to 1 O, up to 1 S and up to 1 N, wherein up to 2 carbon atom ring members are independently selected from C(═O) and C(═S), and the sulfur atom ring members are independently selected from)S(═O)ᵤ(═NR¹⁰)_{z}, the ring optionally substituted with up to 2 substituents independently selected from R^{9a} on carbon atom ring members and from R^{9b} on a nitrogen atom ring member;
each R^{9a} is independently halogen, C₁-C₄ alkyl, C₁-C₄ haloalkyl, C₁-C₄ alkoxy, C₁-C₄ haloalkoxy, C₁-C₄ alkylthio or C₁-C₄ haloalkylthio;
R^{9b} is C₁-C₄ alkyl or C₁-C₄ haloalkyl;
R¹⁰ is independently H, C₁-C₄ alkyl, C₂-C₄ alkenyl, C₂-C₄ alkynyl, C₁-C₄ haloalkyl, C₂-C₄ haloalkenyl, C₂-C₄ haloalkynyl, C₂-C₄ alkoxyalkyl, C₂-C₄ alkylcarbonyl, C₂-C₄ haloalkylcarbonyl, C₁-C₄ alkylsulfonyl or C₁-C₄ haloalkylsulfonyl;
m is 0, 1 or 2;
n is 0, 1 or 2; and
u and z in the instance of)S(═O)ᵤ(═NR¹⁰)_{z} are independently 0, 1 or 2, provided that the sum of u and z in the instance of S(═O)ᵤ(═NR¹⁰)_{z} is 0, 1 or 2;
provided that when X is N, then G is attached to X through a carbon atom ring member.

9. The method of claim 8 wherein
A is O or NH;
R¹ is selected from U-1 through U-51 as shown in Exhibit 1 wherein each R^{V} is independently selected from H and R^{5a} when R^{V} is attached to a carbon atom ring member, and R^{V} is selected from H and R^{5b} when R^{V} is attached to a nitrogen atom ring member, and the bond projecting to the left is bonded to A of Formula 1;
k is 0, 1, 2 or 3;
R⁴ is benzyl, phenyl or naphthalenyl, each optionally substituted with up to 3 substituents independently selected from R^{8a}; or pyridinyl, thienyl, pyrazolyl, triazolyl or imidazolyl, each optionally substituted with up to 3 substituents independently selected from R^{8a} on carbon atom ring members and R^{8b} on a nitrogen atom ring member;
G is selected from G-1 through G-48 as shown in Exhibit 2 wherein R^{Y} is selected from H and R^{7a} when R^{Y} is attached to a carbon atom ring member, and R^{Y} is selected from H and R^{7b} when R^{Y} is attached to a nitrogen atom ring member, and the bond projecting to the left is bonded to X and the bond projecting to the right is bonded to the isoxazole ring in Formula 1; and
q is 0 or 1.

10. The method of claim 9 wherein
A is O;
W is O;
X is CR^{2a};
R¹ is selected from U-21 and U-37 through U-51;
each R² is independently C₁-C₂ alkyl or C₁-C₂ haloalkyl;
R^{2a} is H;
each R³ is independently cyano or C₁-C₃ alkyl;
R⁴ is benzyl or phenyl, each optionally substituted with up to 3 substituents independently selected from R^{8a}; or pyridinyl or thienyl, each optionally substituted with up to 3 substituents independently selected from R^{8a} on carbon atom ring members;
each R^{5a} is independently halogen, hydroxy, cyano, nitro, C₁-C₄ alkyl, C₁-C₆ haloalkyl, C₁-C₄ alkoxy, C₁-C₄ haloalkoxy, C₁-C₄ alkylthio, C₁-C₄ haloalkylthio, C₁-C₄ alkylsulfonyl, C₁-C₄ alkylsulfonyl, C₁-C₄ haloalkylsulfinyl, C₁-C₄ haloalkylsulfonyl, C₂-C₈ dialkylamino, C₂-C₄ alkylcarbonyl, C₂-C₆ alkoxycarbonyl or C₂-C₆ alkylcarbonyloxy;
G is selected from G-25 through G-34 and G-43 through G-48;
each R^{8a} is independently halogen, hydroxy, amino, cyano, nitro, C₁-C₃ alkyl, C₁-C₃ haloalkyl, C₁-C₃ alkoxy, C₁-C₃ haloalkoxy, C₁-C₃ alkylthio or C₁-C₃ haloalkylthio;
n is 0 or 1; and
q is 0.

11. The method of claim 10 wherein
R¹ is selected from U-21, U-37, U-38, U-39, U-42, U-44, U-50 and U-51;
R⁴ is a phenyl optionally substituted with up to 3 substituents independently selected from R^{8a};
each R^{5a} is independently halogen, cyano, nitro, C₁-C₂ alkyl, C₁-C₂ haloalkyl, C₁-C₂ alkoxy or C₁-C₂ haloalkoxy;
n is 0; and
m is 0 or 1.

12. The method of claim 11 wherein
R¹ is selected from U-21, U-50 and U-51;
R³ is cyano or C₁-C₂ alkyl;
each R^{5a} is independently halogen, nitro, C₁-C₂ alkyl, C₁-C₂ haloalkyl or C₁-C₂ alkoxy; and
G is selected from G-26, G-34, G-43 and G-47.

13. The method of claim 12 wherein
R¹ is U-50;
R⁴ is a phenyl;
each R^{5a} is independently bromo, chloro, methyl, trifluoromethyl or methoxy;
G is G-26; and
m is 0.

14. The method of claim 8 wherein the compound is selected from the group:
phenyl 4-[4-(4,5-dihydro-5-phenyl-3-isoxazolyl)-2-thiazolyl]-1-piperidinecarboxylate and
2-chlorophenyl 4-[4-(4,5-dihydro-5-phenyl-3-isoxazolyl)-2-thiazolyl]-1-piperidine-carboxylate.

15. A pharmaceutical composition comprising (a) a compound of Formula 1, an N-oxide or a pharmaceutically acceptable salt thereof as defined in claim 8; and (b) at least one other therapeutic agent.

16. A pharmaceutical composition comprising (a) a compound of Formula 1, an N-oxide or a pharmaceutically acceptable salt thereof as defined in claim 8; and (b) at least one additional component selected from the group consisting of pharmaceutically acceptable carriers.

17. A method of treating a subject for pain, said method comprising administering to the subject in need of such treatment a therapeutically effective amount of an inhibitor of fatty acid amide hydrolase selected from compounds of Formula 1, N-oxides, or pharmaceutically acceptable salts thereof as defined in claim 8.
